(19) United States
(12) Patent Application Publication
(10) Pub. No.: US 2012/0100609 A1
(43) Pub. Date: Apr. 26, 2012

(54) N-LINKED GLYCAN BIOSYNTHESIS MODULATORS

(76) Inventors: Brett E. Crawford, Poway, CA (US); Jillian R. Brown, Poway, CA (US); Charles A. Glass, San Diego, CA (US); Xiaomei Bai, San Diego, CA (US)

(21) Appl. No.: 13/259,099

(22) PCT Filed: Mar. 29, 2010
(86) PCT No.: PCT/US2010/029087

§ 371 Date: Dec. 12, 2011

Related U.S. Application Data
(60) Provisional application No. 61/164,278, filed on Mar. 27, 2009.
(60) Provisional application No. 61/290,457, filed on Dec. 28, 2009.

(51) Int. Cl. C12N 5/071; C07K 14/47; C12N 5/09

(52) U.S. Cl. 435/375; 530/395

(57) ABSTRACT

Provided herein are N-linked glycan inhibitors, including modulators of N-linked glycan glycosylation, mannosidase, an N-linked glycan N-acetylglucosaminyl transferase, an N-linked glycan fucosyl transferase, an N-linked glycan galactosyl transferase, an N-linked glycan sialyl transferase, an N-linked glycan sulfotransferase, N-linked glycan glycophosphotransferase or a combination thereof.

### CROSS-REFERENCE

This application claims the benefit of U.S. Provisional Application No. 61/164,278, filed 27 Mar. 2009, and U.S. Provisional Application No. 61/290,457, filed on 28 Dec. 2009, which applications are incorporated herein by reference.

### STATEMENT AS TO FEDERALLY SPONSORED RESEARCH

Certain inventions described herein were made with the support of the United States government under Contract 1 R21NS057066 by the National Institutes of Health.

### BACKGROUND OF THE INVENTION

N-linked-glycans are found in mammals and comprise a plurality of oligosaccharide chains linked to a core protein via a nitrogen atom of an Asparagine (Asn) residue. The Asn residue occurs in a tripeptide sequence, i.e, a glycosylation sequon, comprising e.g. Asn-X-Ser, Asn-X-Thr or Asn-X-Cys, wherein X is an amino acid other than proline.

### SUMMARY OF THE INVENTION

Provided in certain embodiments, herein is a process for modifying the structure of a N-linked glycan on a core protein, comprising contacting a cell that translationally produces at least one core protein having at least one attached N-linked glycan moiety with a selective inhibitor of N-linked glycan biosynthesis, including a mannosidase (e.g., a selective inhibitor of a mannosidase), an N-linked glycan N-acetylglucosaminyl transferase (e.g., a selective inhibitor of an N-linked glycan N-acetylglucosaminyl transferase), an N-linked glycan fucosyl transferase (e.g., a selective inhibitor of an N-linked glycan fucosyl transferase), an N-linked glycan galactosyl transferase (e.g., a selective inhibitor of an N-linked glycan galactosyl transferase), an N-linked glycan sialyl transferase (e.g., a selective inhibitor of an N-linked glycan sialyl transferase), an N-linked glycan sulfotransferase (e.g., a selective inhibitor of an N-linked glycan sulfotransferase), or an N-linked glycan glycophosphotransferase (e.g., a selective inhibitor of an N-linked glycan glycophosphotransferase) or a combination thereof. In some embodiments, the inhibitor of N-linked glycan biosynthesis is an inhibitor of late-stage N-linked glycan biosynthesis (e.g., a selective inhibitor of late-stage N-linked glycan biosynthesis).

Provided in some embodiments herein is a process for modifying the population of N-linked glycans on one or more proteins associated with a cell, the process comprising contacting a cell that produces N-linked glycans with an effective amount of a selective late stage N-linked glycan biosynthesis inhibitor, the selective late stage N-linked glycan biosynthesis inhibitor being active in a mammalian cell. In certain embodiments, the selective late stage N-linked glycan biosynthesis inhibitor utilized in any process described herein is active in a mammalian cell and is a non-carbohydrate inhibitor. In some embodiments, the selective N-linked glycan biosynthesis inhibitor utilized in any process herein has a molecular weight of less than 700 g/mol.

In certain embodiments, any process described herein reduces the ratio of complex N-linked glycans to high mannose N-linked glycans. In a specific embodiments, the process reduces the amount of tri-antennary and tetra-antennary N-linked glycans in the cellular population of N-linked glycan. In another specific embodiment, the process reduces the cellular population of (β1-6) branching N-linked glycans. In yet another specific embodiment, the process reduces the cellular population of poly-N-acetyllactosamine-containing N-glycans. In a further specific embodiment, the process reduces the cellular population of outer-chain polyfucosylation and/or sialyl Lewis X containing N-glycans.

The some embodiments, the selective N-linked glycan biosynthesis inhibitor utilized in any process described herein inhibits GlcNAc-T-V, GlcNAc-T-IV, GlcNAc-T-III, GlcNAc-T-II, or a combination thereof. In a specific embodiment herein, the selective N-linked glycan biosynthesis inhibitor utilized in any process described herein indirectly inhibits GlcNAc-T-V, GlcNAc-T IV GlcNAc-T-III, GlcNAc-T-II, or a combination thereof. In another specific embodiment, the selective N-linked glycan biosynthesis inhibitor utilized in any process described herein directly inhibits GlcNAc-T-V, GlcNAc-T-IV, GlcNAc-T-III, GlcNAc-T-II, or a combination thereof.

In certain embodiments, the selective N-linked glycan biosynthesis inhibitor utilized in any process described herein inhibits modifications including (α2,3) sialylation, (α2,6) sialylation, (α1,3) fucosylation, 6-sulfation of the terminal galactose, 6-sulfation of the penultimate GlcNAc.

In some embodiments, the cell contacted by any process described herein is an inflammatory cell, a cancer cell, an endothelial cell, a cell having abnormal N-linked glycan accumulation, or a cell susceptible to viral and pathogenic infection. In certain embodiments, the cell contacted by any process described herein is present in an individual diagnosed with or suspected of having rheumatoid arthritis, Crohn's disease, or inflammatory bowel disease, lung cancer, colon cancer, breast cancer, pathogenic angiogenesis. In some embodiments, the cell contacted by any process described herein is present in an individual diagnosed with or suspected of having influenza or HIV, or a lysosomal storage disease (including Sandhoff, Tay Sachs, GM1 gangliosidosis).

In certain embodiments, disclosed herein is an N-linked glycanated protein comprising a core protein covalently linked to at least one N-linked glycan, wherein the at least one N-linked glycan comprises a plurality of high mannose, hybrid or complex N-linked glycan structures, and wherein less than 9% (mole percentage), less than 8% (mole percentage), less than 7% (mole percentage), less than 6% (mole percentage), less than 5% (mole percentage), less than 4% (mole percentage), less than 3% (mole percentage), less than 2% (mole percentage), less than 1% (mole percentage), less than 0.5% (mole percentage) of the plurality of high mannose, hybrid or complex N-linked glycan structures are tri-antennary N-linked glycans.

In some embodiments, disclosed herein is an N-linked glycanated protein comprising a core protein covalently linked to at least one N-linked glycan, wherein the at least one N-linked glycan comprises a plurality of high mannose, hybrid or complex N-linked glycan structures, and wherein less than 2% (mole percentage), less than 1.5% (mole percentage), less than 1% (mole percentage), less than 0.5% (mole percentage), less than 0.2% (mole percentage), less than 0.1% (mole percentage) of the plurality of high mannose, hybrid or complex N-linked glycan structures are tetra-antennary N-linked glycans.

In certain embodiments, disclosed herein is an N-linked glycanated protein comprising human serum acid alpha-1-glycoprotein N-linked glycanated with bi-antennary, tri-antennary and tetra-antennary N-linked glycans, wherein less than 52% (w/w), less than 51% (w/w), less than 50% (w/w), less than 40% (w/w), less than 30% (w/w), less than 20% (w/w), less than 10% (w/w), less than 5% (w/w), less than 2.5% (w/w) are tri-antennary and tetra-antennary N-linked glycans.

In some embodiments, disclosed herein is an N-linked glycanated protein comprising human serum acid alpha-1-glycoprotein N-linked glycanated with bi-antennary, tri-antennary and tetra-antennary N-linked glycans, wherein less than 12% (w/w), less than 11% (w/w), less than 10% (w/w), less than 5% (w/w), less than 2% (w/w), less than 1% (w/w), less than 0.5% (w/w) are tetra-antennary.

Described in certain embodiments herein is a process for modifying the structure of an N-linked glycan on a core protein, the process comprising contacting a cell that translationally produces at least one core protein having at least one attached N-linked glycan moiety with an effective amount of a selective inhibitor of an N-linked glycan N-acetylglucosaminyl transferase.

In one embodiment the selective inhibitor of the N-linked glycan N-acetylglucosaminyl transferase is an inhibitor of N-acetylglucosaminyl transferase I, N-acetylglucosaminyl transferase II, N-acetylglucosaminyl transferase III, N-acetylglucosaminyl transferase IV, N-acetylglucosaminyl transferase V, or a combination thereof. In another embodiment the inhibitor of N-acetylglucosaminyl transferase V inhibits the addition of N-acetylglucosamine to an N-linked glycan via a β1,6 linkage. In yet another embodiment the selective inhibitor of N-acetylglucosaminyl transferase I or N-acetylglucosylaminyl transferase II inhibits the addition of N-acetylglucosamine to an N-linked glycan via a β1,2 linkage. In a further embodiment the inhibitor of N-acetylglucosaminyl transferase III inhibits the addition of N-acetylglucosamine to an N-linked glycan via a β1,4 linkage. In yet a further embodiment the inhibitor of N-acetylglucosaminyl transferase IV inhibits the addition of N-acetylglucosamine to an N-linked glycan via a β1,4 linkage.

In one embodiment the cell being contacted is a cell in need thereof, a cell present in an individual suffering from a disease or condition mediated by abnormal N-linked glycan biosynthesis and/or the cell itself is a cell with abnormal N-linked glycan biosynthesis, a cell present in an individual with normal N-linked glycan biosynthesis and/or the cell itself is a cell with normal N-linked glycan biosynthesis. In some embodiments, the cell being contacted is a cell present in an individual with normal N-linked glycan biosynthesis (e.g., an individual with a predisposition for or suspected of having a disease or condition mediated by N-linked glycan biosynthesis) and/or the cell itself is a cell with normal N-linked biosynthesis.

Also presented herein is a process for inhibiting the synthesis of a β1,6 linked glycan, the process comprising contacting a cell having at least one core protein attached to at least one pentasaccharide core with an effective amount of a selective inhibitor of an N-acetylglucosaminyl transferase.

In one embodiment the pentasaccharide core has the Formula:

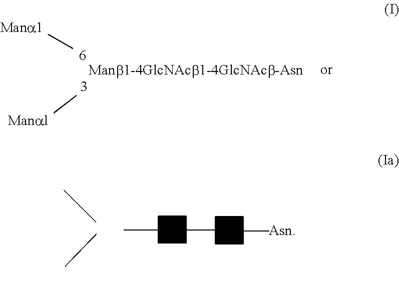

In another embodiment the N-acetylglucosaminyl transferase is N-acetylglucosaminyl transferase V. In one embodiment the N-acetylglucosaminyl transferase is N-acetylglucosaminyl transferase I. In one embodiment the N-acetylglucosaminyl transferase is N-acetylglucosaminyl transferase III. In one embodiment the N-acetylglucosaminyl transferase is N-acetylglucosaminyl transferase IV. In one embodiment inhibiting the synthesis of the β1,6 linked glycan inhibits the formation of a complex β1,6 branched N-linked glycan.

Presented herein is a process of modifying the structure of a complex N-linked glycan on a core protein, the process comprising contacting a cell that translationally produces at least one core protein having at least one attached complex N-linked glycan moiety with an effective amount of a selective inhibitor of an N-linked glycan N-acetylglucosaminyl transferase.

In one embodiment the selective inhibitor of the N-linked glycan N-acetylglucosaminyl transferase is an inhibitor of an N-acetylglucosoaminyl transferase IV or an N-acetylglucosaminyl transferase V or a combination thereof.

Also disclosed herein is a process of inhibiting the formation of a complex N-linked glycan on a core protein, comprising contacting a cell that translationally produces at least one core protein having at least one attached hexasaccharide moiety with an effective amount of a selective inhibitor of an N-linked glycan N-acetylglucosaminyl transferase.

In one embodiment the at least one attached hexasaccharide moiety has three N-acetylglucosamine residues and three mannose residues. In another embodiment the complex N-linked glycan is a di-antennary N-linked glycan. In another embodiment the complex N-linked glycan is a tri-antennary N-linked glycan. In yet another embodiment the complex N-linked glycan is a tetra-antennary N-linked glycan. In a further embodiment the selective inhibitor of the N-linked glycan N-acetylglucosaminyl transferase is an inhibitor of N-acetylglucosaminyl transferase III. In yet a further embodiment the selective inhibitor of the N-linked glycan N-acetylglucosaminyl transferase is an inhibitor of N-acetylglucosaminyl transferase IV. In another embodiment the selective inhibitor of the N-linked glycan N-acetylglucosaminyl transferase is an inhibitor of N-acetylglucosaminyl transferase V.

Described herein is a process of inhibiting the formation of a hybrid N-linked glycan on a core protein, comprising contacting a cell that translationally produces at least one core protein having at least one attached pentasaccharide core with an effective amount of a selective inhibitor of an N-linked glycan N-acetylglucosaminyl transferase.

In yet a further embodiment the selective inhibitor of the N-linked glycan N-acetylglucosaminyl transferase is an inhibitor of N-acetylglucosaminyl transferase I.

Also presented herein is a method of treating cancer in a subject comprising administering to the subject a therapeutically effective amount of a selective modulator of an N-linked glycan N-acetylglucosaminyl transferase.

In one embodiment the selective modulator of an N-linked glycan N-acetylglucosaminyl transferase is a modulator of N-acetylglucosaminyl transferase V. In another embodiment the cancer is selected from breast carcinoma and colorectal carcinoma.

In yet another aspect is a process for modifying the structure of an N-linked glycan on a core protein, comprising contacting a cell that translationally produces at least one core protein having at least one attached N-linked glycan moiety with an effective amount of a selective inhibitor of N-linked glycan biosynthesis. In another embodiment the selective inhibitor reduces or inhibits the activity of a mannosidase, an N-linked glycan fucosyl transferase, an N-linked glycan galactosyl transferase, an N-linked glycan sialyl transferase, an N-linked glycan sulfotransferase, or N-linked glycan glycophosphotransferase or a combination thereof. In yet another embodiment the selective inhibitor of an N-linked glycan mannosidase is an inhibitor of a Golgi mannosidase I or a Golgi mannosidase II or a combination thereof. In a further embodiment the inhibitor of the Golgi mannosidase I inhibits the cleavage of two mannose residues from a Man(α1,3) branch. In yet a further embodiment the inhibitor of the Golgi mannosidase II inhibits the cleavage of two mannose residues from a Man(α1,6) branch. In some instances, the selective inhibitor of N-linked glycan biosynthesis is not castanospermine, 1,6-epicyclophellitol or amphomycin. In some instances, the selective inhibitor of N-linked glycan biosynthesis is not 1-deoxynojirmycin, N-methyl-1-deoxynojirimycin, mannostatin or swainsonine. In one embodiment the inhibitor of the N-linked glycan fucosyl transferase inhibits the addition of a fucose residue via an α1,6 linkage. In another embodiment the inhibitor of the N-linked glycan fucosyl transferase inhibits the addition of a fucose residue via an α1,3 linkage. In yet another embodiment the inhibitor of the N-linked glycan galactosyl transferase inhibits the addition of a galactose residue via a β1,4 linkage. In a further embodiment the inhibitor of the N-linked glycan galactosyl transferase inhibits the addition of a galactose residue via an α1,3 linkage or a β1,3 linkage. In yet a further embodiment the inhibitor of the N-linked glycan sialyl transferase inhibits the addition of a sialic acid residue via an α2,6 linkage or an α2,3 linkage. In one embodiment the inhibitor of the N-linked glycan sialyl transferase inhibits the addition of a sialic acid residue to a preceding sialic acid via an α2,8 linkage. In another embodiment modifying the structure of an N-linked glycan on the core protein further comprises contacting the cell with a selective inhibitor of an N-linked glycan N-acetylglucosaminyl transferase. In yet another embodiment the selective inhibitor of the N-linked glycan N-acetylglucosaminyl transferase is an inhibitor of N-acetylglucosaminyl transferase V.

In some embodiments, the cell being contacted is a cell in need thereof, a cell present in an individual suffering from a disease or condition mediated by abnormal N-linked glycan biosynthesis and/or the cell itself is a cell with abnormal N-linked glycan biosynthesis, a cell present in an individual with normal N-linked glycan biosynthesis and/or the cell itself is a cell with normal N-linked glycan biosynthesis. In some embodiments, the cell being contacted is a cell present in an individual with normal N-linked glycan biosynthesis (e.g., an individual with a predisposition for or suspected of having a disease or condition mediated by N-linked glycan biosynthesis) and/or the cell itself is a cell with normal N-linked biosynthesis.

In another aspect is a method of treating cancer in a subject comprising administering to the subject a therapeutically effective amount of a selective modulator of N-linked glycan biosynthesis. In yet another embodiment the selective inhibitor reduces or inhibits the activity of a mannosidase, an N-linked glycan fucosyl transferase, an N-linked glycan galactosyl transferase, an N-linked glycan sialyl transferase, an N-linked glycan sulfotransferase, or N-linked glycan glycophosphotransferase or a combination thereof. In another embodiment the selective modulator of the N-linked glycan mannosidase is an inhibitor of a Golgi mannosidase I or a Golgi mannosidase II or a combination thereof. In yet another embodiment the inhibitor of the Golgi mannosidase I inhibits the cleavage of two mannose residues from a Man(α1,3) branch. In a further embodiment the inhibitor of the Golgi mannosidase II inhibits the cleavage of two mannose residues from a Man(α1,6) branch. In some instances, the selective modulator of N-linked glycan biosynthesis is not 1-deoxymannojirimycin, mannostatin or swainsonine. In yet a further embodiment the selective modulator of the N-linked glycan fucosyl transferase inhibits the addition of a fucose residue via an α1,6 linkage. In one embodiment the selective modulator of the N-linked glycan fucosyl transferase inhibits the addition of a fucose residue via an α1,3 linkage. In another embodiment the selective modulator of the N-linked glycan galactosyl transferase inhibits the addition of a galactose residue via a β1,4 linkage. In yet another embodiment the selective modulator of the N-linked glycan galactosyl transferase inhibits the addition of a galactose residue via an α1,3 linkage or a β1,3 linkage. In a further embodiment the selective modulator of the N-linked glycan sialyl transferase inhibits the addition of a sialic acid residue via an α2,6 linkage or an α2,3 linkage. In yet a further embodiment the selective modulator of the N-linked glycan sialyl transferase inhibits the addition of a sialic acid residue to a preceding sialic acid via an α2,8 linkage.

Also provided herein is a method of treating a lysosomal storage disease in a subject comprising administering to the subject a therapeutically effective amount of a selective modulator of N-glycan biosynthesis or N-glycan degradation. In some embodiments, the lysosomal storage disease is selected from mucopolysaccharidosis.

Also described herein is a process of inhibiting N-linked glycan function in a cell, the process comprising contacting the cell with an effective amount of a selective modulator of N-linked glycan biosynthesis.

In one embodiment the selective modulator of N-linked glycan biosynthesis reduces or inhibits the activity of a mannosidase, an N-linked glycan N-acetylglucosaminyl transferase, an N-linked glycan fucosyl transferase, an N-linked glycan galactosyl transferase, an N-linked glycan sialyl transferase, an N-linked glycan sulfotransferase, or N-linked glycan glycophosphotransferase or a combination thereof. In one embodiment the N-linked glycan function inhibited is an ability to bind an N-linked glycan binding lectin. In another embodiment the N-linked glycan is modified with N-acetylactosamine. In yet another embodiment the N-linked glycan binding lectin is galectin-3 or any one or more galectin. In a further embodiment the N-linked glycan function inhibited is an ability to bind a growth factor. In yet a further embodiment the growth factor is a fibroblast growth factor (FGF) epidermal growth factor or transforming growth factor-β receptors. In one embodiment the N-linked glycan function inhibited is the function of an N-linked glycanated protein e.g., of an integrin, a matriptase or N-cadherin (such as the binding or signaling thereof).

In some embodiments, the cell being contacted is a cell in need thereof, a cell present in an individual suffering from a disease or condition mediated by abnormal N-linked glycan biosynthesis and/or the cell itself is a cell with abnormal N-linked glycan biosynthesis, a cell present in an individual with normal N-linked glycan biosynthesis and/or the cell itself is a cell with normal N-linked glycan biosynthesis. In some embodiments, the cell being contacted is a cell present in an individual with normal N-linked glycan biosynthesis (e.g., an individual with a predisposition for or suspected of having a disease or condition mediated by N-linked glycan biosynthesis) and/or the cell itself is a cell with normal N-linked biosynthesis.

In another aspect is a process of normalizing and/or modulating the biosynthesis of an N-linked glycan on a core protein in a subject suffering from abnormal N-linked glycan biosynthesis comprising administering to the subject a therapeutically effective amount of an agent that reduces or inhibits the activity of an upstream regulator of the N-linked glycan.

In one embodiment the agent is a selective inhibitor of an oligosaccharyltransferase, a glucosidase, a mannosidase, or a combination thereof. In another embodiment the selective inhibitor of the glucosidase is an inhibitor of an α1,2-glucosidase I (e.g., a selective inhibitor of an α1,2-glucosidase I) or an inhibitor of inhibitor of an α1,3-glucosidase II (e.g., a selective inhibitor of an α1,3-glucosidase II) or a combination thereof. In yet another embodiment the selective inhibitor of the mannosidase is an inhibitor of an endoplasmic reticulum α1,2-mannosidase (e.g., a selective inhibitor of an endoplasmic reticulum α1,2-mannosidase). In a further embodiment the agent is not tunicamycin or amphomycin. In yet a further embodiment the agent is not 1-deoxynojirmycin or N-methyl-1-deoxynojirimycin.

In some embodiments, the agent is or does not comprise a carbohydrate. In some embodiments, the agent is a small molecule. In some embodiments, the agent is a non-carbohydrate small molecule.

Provided herein is a process for identifying a compound that modulates N-linked glycan biosynthesis comprising:
a. contacting a mammalian cell with the compound
b. contacting the mammalian cell and compound combination with a first labeled probe wherein the first labeled probe binds one or more N-linked glycans;
c. incubating the mammalian cell, compound, and the first labeled probe;
d. collecting the first labeled probe that is bound to one or more N-linked glycans; and
e. detecting or measuring the amount of first labeled probe bound to one or more N-linked glycans.

Further provided herein is a process for identifying a compound that selectively modulates N-linked glycan biosynthesis comprising:
a. contacting a mammalian cell with the compound
b. contacting the mammalian cell and compound combination with a first labeled probe and a second labeled probe, wherein the first labeled probe binds one or more N-linked glycans and the second labeled probe binds at least one glycan other than N-linked glycans;
c. incubating the mammalian cell, compound, the first labeled probe, and the second labeled probe;
d. collecting the first labeled probe that is bound to one or more N-linked glycans;
e. collecting the second labeled probe that is bound to at least one glycan other than N-linked glycans;
f. detecting or measuring the amount of first labeled probe bound to one or more N-linked glycans; and
g. detecting or measuring the amount of the second labeled probe bound to at least one glycan other than N-linked glycans.

In some embodiments, the mammalian cell is a human cancer cell. In some embodiments, the labeled probe comprises a biotinyl moiety and the process further comprises tagging the labeled probe with streptavidin-Cy5-PE. In some embodiments, the labeled probe comprises a fluorescent label. In some embodiments, the first labeled probe is a labeled protein. In some embodiments, the labeled protein is a N-linked glycan-specific lectin. In some embodiments, the second labeled probe is a labeled lectin. In some embodiments, the labeled lectin is a lectin that is specific for a glycan other than a N-linked glycan.

Provided herein is an N-linked proteoglycan comprising a core protein covalently linked to at least one N-linked glycan, wherein the at least one N-linked glycan comprises a plurality of high mannose, hybrid or complex N-linked glycan structures, and wherein less than 20% of the plurality of high mannose, hybrid or complex N-linked glycan structures are di-antennary N-linked glycans, tri-antennary N-linked glycans or tetra-antennary N-linked glycans.

Also provided herein is an N-linked proteoglycan comprising a core protein covalently linked to at least one N-linked glycan, wherein the at least one N-linked glycan comprises a plurality of high mannose, hybrid or complex N-linked glycan structures, and wherein less than 10% of the plurality of high mannose, hybrid or complex N-linked glycan structures are di-antennary N-linked glycans, tri-antennary N-linked glycans or tetra-antennary N-linked glycans.

Other objects and features of the methods, compositions and uses described herein will become apparent from the following detailed description. It should be understood, however, that the detailed description and the specific examples, while indicating some embodiments, are given by way of illustration only.

### BRIEF DESCRIPTION OF THE DRAWINGS

The features of the present disclosure are set forth with particularity in the appended claims. A better understanding of the features and advantages of the present disclosure are obtained by reference to the following detailed description that sets forth illustrative embodiments, in which the principles of the embodiments are utilized, and the accompanying drawings of which:

FIG. 1 illustrates the different structures that the lectins (ConA, RCA, and L-PHA) bind to with high affinity.

FIG. 2 illustrates Flow cytometry showing specific binding of the lectin Phaseolus vulgaris Lekoagglutinin (L-PHA) which binds to complex-type N-glycans with α1-6 mannose substituted branches.

FIG. 3 illustrates Phase I N-linked glycan biosynthesis (synthesis of dolichol-P-P-GlcNAc2Man9Glc3).

FIG. 4 illustrates Phase II N-linked glycan biosynthesis (the processing and maturation of an N-glycan).

FIG. 5 illustrates a portion of Phase III N-linked glycan biosynthesis (the processing and maturation of an N-glycan).

FIG. 6 illustrates a portion of Phase III N-linked glycan biosynthesis (the branching of complex N-glycans).

FIG. 7 illustrates a portion of Phase III N-linked glycan biosynthesis (modifications of the core of N-glycans).

FIG. 8 illustrates a portion of Phase III N-linked glycan biosynthesis (elongation of branch N-acetylglucosamine residues of N-glycans).

FIG. 9 illustrates exemplary complex N-glycan structures found on mature glycoproteins.

FIG. 10 illustrates the affects of compound 1 on the ability of the lectin Phaseolus Vulgaris Agglutinin type L (PHA) to bind to treated and untreated Chinese Hamster Ovary (CHO) cells.

FIG. 11 illustrates the affects of compound 2 on the ability of the lectin Phaseolus Vulgaris Agglutinin type L (PHA) to bind to treated and untreated Chinese Hamster Ovary (CHO) cells.

FIG. 12 illustrates the affects of compound 3 on the ability of the lectin Phaseolus Vulgaris Agglutinin type L (PHA) to bind to treated and untreated Chinese Hamster Ovary (CHO) cells.

FIG. 13 illustrates the affects of compound 4 on the ability of the lectin Phaseolus Vulgaris Agglutinin type L (PHA) to bind to treated and untreated Chinese Hamster Ovary (CHO) cells.

FIG. 14 illustrates the affects of compound 5 on the ability of the lectin Phaseolus Vulgaris Agglutinin type L (PHA) to bind to treated and untreated Chinese Hamster Ovary (CHO) cells.

FIG. 15 illustrates the affects of compound 6 on the ability of the lectin Phaseolus Vulgaris Agglutinin type L (PHA) to bind to treated and untreated Chinese Hamster Ovary (CHO) cells.

FIG. 16 illustrates the affects of compound 7 on the ability of the lectin Phaseolus Vulgaris Agglutinin type L (PHA) to bind to treated and untreated Chinese Hamster Ovary (CHO) cells.

FIG. 17 illustrates the specificity of compound 8 by probing with PHA and with fibroblast growth factor 2 (FGF2).

FIG. 18 illustrates the specificity of compound 9 by probing with PHA and with fibroblast growth factor 2 (FGF2).

FIG. 19 illustrates the specificity of compound 10 by probing with PHA and with fibroblast growth factor 2 (FGF2).

FIG. 20 illustrates the specificity of compound 11 by probing with PHA and with fibroblast growth factor 2 (FGF2).

FIG. 21 illustrates the specificity of compound 12 by probing with PHA and with fibroblast growth factor 2 (FGF2).

FIG. 22 illustrates the specificity of compound 13 by probing with PHA and with fibroblast growth factor 2 (FGF2).

FIG. 23 shows the effect of inhibiting a Phase I “trimming” enzyme with Castanospermine. Castanosperime inhibits the glucosidase I and II required to get past high mannose N-linked glycans.

FIG. 24 shows Castanospermine Treated CHO cells with N-glycan Profile Changes.

FIG. 25 shows HPLC profiles in experiments using compound 10 as an inhibitor. The peaks are labeled with their retention times and the identities of certain peaks indicated where they could be determined from the standards. Other peaks are identified by glucose units (GU) which were determined by extrapolation by comparing their retention times to the retention times of peaks in the glucose oligomer ladder. Compound doses are in uM. Ctrl=0 compound. EU=Fluorescence excitation units.

FIG. 26 shows graphs illustrating the increases and decreases of specific peaks in response to treatment with compound 10. The peaks are identified by their position on the profile in glucose units (GU) and where possible the peaks are identified by comparison with the standards. Peaks are identified on the HPLC profiles by arrows. Compound doses are in uM. The Y-axis shows the peak areas at the different compound doses as the % of the untreated Cntr peak area.

FIG. 27 shows HPLC profiles in experiments using compound 12 as an inhibitor. The peaks are labeled with their retention times and the identities of certain peaks indicated where they could be determined from the standards. Other peaks are identified by glucose units (GU) which were determined by extrapolation by comparing their retention times to the retention times of peaks in the glucose oligomer ladder. Compound doses are in uM. Ctrl=0 compound. EU=Fluorescence excitation units.

FIG. 28 shows graphs illustrating the increases and decreases of specific peaks in response to treatment with compound 12. The peaks are identified by their position on the profile in glucose units (GU) and where possible the peaks are identified by comparison with the standards. Peaks are identified on the HPLC profiles by arrows. Compound doses are in uM. The Y-axis shows the peak areas at the different compound doses as the % of the untreated Cntr peak area.

FIG. 29 shows HPLC profiles in experiments using compound 11 as an inhibitor. The peaks are labeled with their retention times and the identities of certain peaks indicated where they could be determined from the standards. Other peaks are identified by glucose units (GU) which were determined by extrapolation by comparing their retention times to the retention times of peaks in the glucose oligomer ladder. Compound doses are in uM. Ctrl=0 compound. EU=Fluorescence excitation units.

FIG. 30 shows graphs illustrating the increases and decreases of specific peaks in response to treatment with compound 11. The peaks are identified by their position on the profile in glucose units (GU) and where possible the peaks are identified by comparison with the standards. Peaks are identified on the HPLC profiles by arrows. Compound doses are in uM. The Y-axis shows the peak areas at the different compound doses as the % of the untreated Cntr peak area.

FIGS. 31A-31T illustrate selective N-linked glycan biosynthesis inhibitors according to certain embodiments.

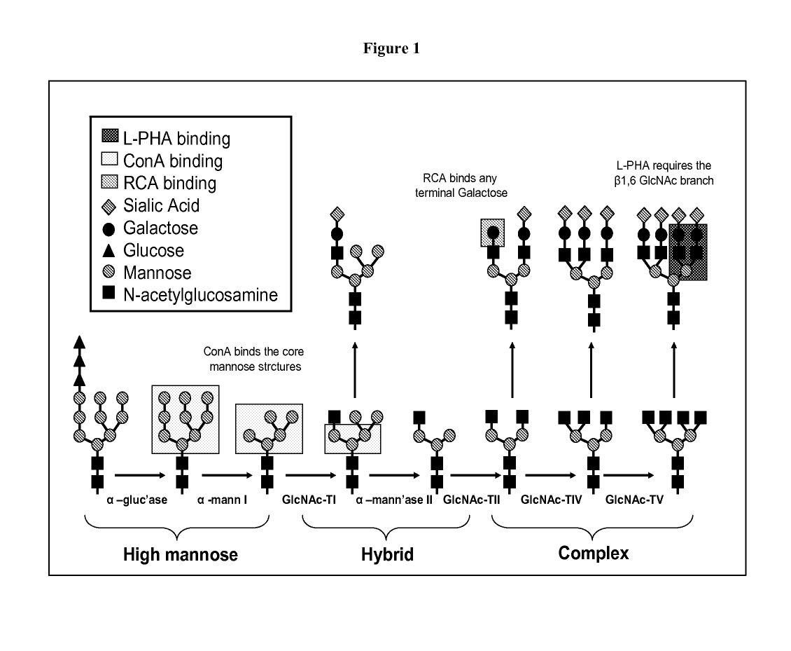

### DETAILED DESCRIPTION OF THE INVENTION

### N-Linked Glycan Synthesis Inhibitors

Provided in certain embodiments herein are N-linked glycan synthesis inhibitors. In general, N-linked glycan synthesis inhibitors modulate or alter the nature (e.g., character, structure, or concentration) of N-linked glycans (e.g., N-linked glycans on a protein or biomolecule, or in a cell, tissue, organ or individual). N-linked glycans present on glycoproteins comprise a plurality of oligosaccharide chains attached to a core protein via a nitrogen atom of an Asn residue. The Asn residue occurs in a tripeptide sequence, i.e, a glycosylation sequon, comprising e.g. Asn-X-Ser, Asn-X-Thr or Asn-X-Cys, wherein X is an amino acid other than proline.

The synthesis of N-linked glycans is preceded by the biosynthesis of a 14-residue oligosaccharide precursor molecule in the cytosolic site of the rough endoplasmic reticulum (RER). The synthesis of the 14-residue precursor is initiated by the transfer of GlcNAc from UDP-GlcNAc to dolichol (Dol-P). In some instances the precursor synthesis is initiated by a UDP-GlcNAc transferase. The GlcNAc unit attached to Dolichol is further polymerized with oligosaccharide units to a (Manα/β)₅-(GlcNAcβ)₂-DolP unit. In some instances the polymerization of saccharide units is mediated by mannosyl transferases (e.g., GDP mannosyl transferase). The (Manα/β)₅-(GlcNAcβ)₂-DolP unit is further polymerized in the lumen of the RER to a 14 residue precursor, i.e., a (Glcα)₃-(Man α/β)₉-(GlcNAcβ)₂-DolP unit (precursor unit). In some instances, attachment of the precursor unit to an Asparagine residue on a protein (i.e., synthesis of (Glcα)₃-(Man α/β)₉-(GlcNAcβ)₂-Asn) is mediated by an oligosaccharyl transferase (e.g., Dolichol-OST). In various embodiments, one or more of the mannose residues (Man) of any of the glycans or units described herein are optionally phosphorylated. Dol-P is released upon attachment of the precursor unit to an Asparagine residue on a protein.

In some instances, the attachment of the 14-residue precursor to an Asn residue on a core protein is followed by further processing of the precursor unit by glucosidases (e.g., α-1,2-glucosidase I, α-1,3-glucosidase II) that cleave terminal Glc residues. In some instances, the terminal Glc residue of the 14-residue precursor unit is removed by α-1,2-glucosidase I. In some instances, the remaining two Glc residues are cleaved by α-1,3-glucosidase II. In some instances, after removal of Glc residues, a mannose residue (e.g. a mannose on a (α-1,6) branch) is cleaved by a mannosidase (e.g., α-1,2-manosidase). In certain instances, a Golgi mannosidase I (e.g., α-1,2 specific) cleaves 2 Man residues from the Man(α1,3) branch to yield (Man α/β)₅-(GlcNAc_{β})₂. In certain instances, a Golgi α-mannosidase II cleaves 2 Man residues from a Man(α1,6). The glycoprotein with the linked (Man α/β)₈-(GlcNAcβ)₂-Asn unit is further processed, e.g., glycophosphorylation by N-acetylglucosaminyl-phosphotransferase and/or removal of NAcGlc by action of N-acetylglucosamine-1-phosphodiester alpha-N-acetylglucosaminidase. In some instances, (Man α/β)₆-(GlcNAcβ)₂-Asn interacts with a corresponding receptor for transport to lysosomes, wherein one or more of the Man are optionally phosphorylated.

In some instances, Man residues are successively cleaved from a high mannose type precursor to yield cores for the synthesis of complex and hybrid type oligosaccharide side chains. In certain instances, successive cleavage of mannose residues yields a pentasaccharide core of the Formula I or Formula Ia:

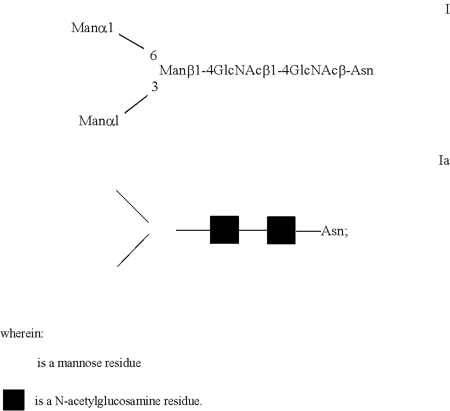

In some embodiments, glycoproteins that comprise N-linked glycans contain a plurality of different sub-types of N-linked glycans (e.g., high mannose, complex or hybrid N-linked glycans). Multiple N-glycosylation sites on the same protein contain different glycan structures, i.e., there is microheterogeneity amongst N-glycans. Within the class of N-linked glycans, there is broad variability with respect to the location and degree of glycosylation and other modifications of the N-linked glycans present on a core protein. In certain instances, a high mannose N-linked glycan is formed when mannose residues are polymerized to a pentasaccharide core as shown below:

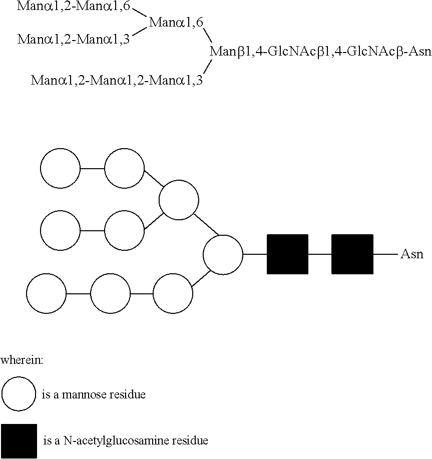

In certain instances, a GlcNAc transferase (e.g., GlcNAc-TI) links a GlcNAc residue β-1,6 to the pentasaccharide core. Further polymerization is mediated by a series of GlcNAc-Ts (e.g., GlcNAc-TII, GlcNAc-TIV, and/or GlcNAc-TV) and forms a complex N-linked glycan as shown below.

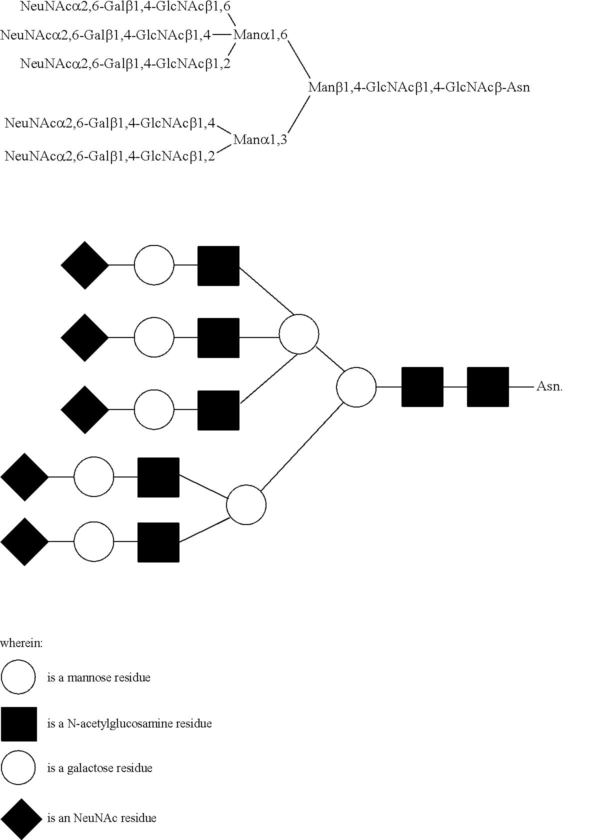

In certain instances, an N-acetylglucosaminyltransferase I (GlcNAc-T1) links a GlcNAc residue β-1,2 to the terminal Man residue of the Man(α1,3) branch of the pentasaccharide core. In some instances, N-acetylglucosaminyl-transferase III (GlcNAcT-III) acts on a hybrid N-glycan (e.g., GlcNAc₁Man₅GlcNAc₂-Asn) and introduces a bisecting GlcNAc unit on the glycan, and if it does, α-mannosidase II cannot cleave the two outer mannose residues; thus, the N-glycan remains of the “unprocessed hybrid” subtype as shown below. In other instances, in the absence of GlcNAcT-III, an α1,3/6 mannosidase acts on a hybrid N-glycan GlcNAc₁Man₅GlcNAc₂-Asn and cleaves the two outer mannose residues. The N-glycan is then further processed to a complex glycan as shown above.

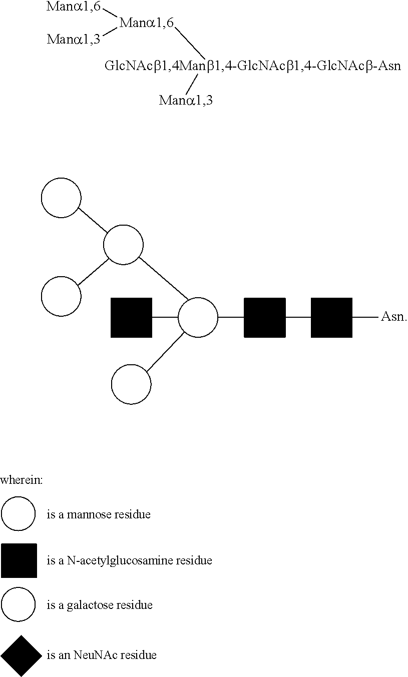

In some instances, an N-linked glycan comprises a keratan linkage saccharide (e.g., Galβ1-4GlcNAcβ1-Galβ1-3GalNAcβ1-N-Asn). In some instances, any of the complex N-linked glycans described herein are optionally and independently modified to complex N-linked glycans with two (e.g., di-antennary N-linked glycans), three (e.g., tri-antennary N-linked glycans) or four branches (e.g., tetra-antennary N-linked glycans). In some instances any of the N-linked glycans described herein (e.g., high mannose, complex, hybrid, di-antennary tri-antennary, tetra-antennary) are optionally and independently further modified. In some instances further modification includes fucosylation, e.g., a fucosyl residue is linked (e.g., α1,6, α1,3) to an N-linked glycan by a fucosyltransferase (e.g., α1,6 fucosyltransferase). In some instances further modification includes sialylation, e.g., a sialic acid residue is linked to an N-linked glycan (e.g., α2,6 or α2,3 to a Gal residue) by a sialyltransferase (e.g., α2,3 sialyltransferase (e.g., ST3Gal IV, ST3Gal VI)). In some instances, a sialic acid residue is linked α2,8 to another preceding sialic acid. In certain instances, further modification includes polymerization, e.g., polylactosamine oligosaccharide chains are linked to N-linked glycans (e.g. to Manα-1,6 or to Man α-1,3 of a pentasaccharide core) by e.g., i-extension enzymes (i-GnT), β-1,3 N-acetylglucosaminyltransferases or β1-4-galactosyltransferases (e.g. β4Gal-TIV).

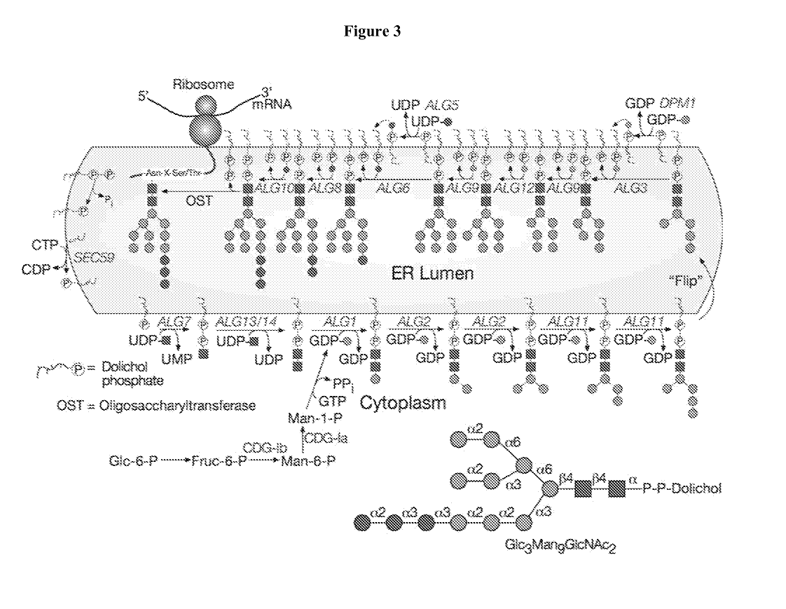

FIG. 3 illustrates Phase I of a N-linked glycan biosynthesis (a synthesis of dolichol-P-P-GlcNAc2Man9G1c3) that occurs in certain instances. Dolichol (squiggle) phosphate (Dol-P) located on the cytoplasmic face of the ER membrane receives GlcNAc-1-P from UDP-GlcNAc in the cytoplasm to generate Dol-P-P-GlcNAc. Dol-P-P-GlcNAc is extended to Dol-P-P-GlcNAc2Man5 before being “flipped” across the ER membrane to the lumenal side according to a process illustrated in FIG. 3. FIG. 3 further illustrates that on the lumenal face of the ER membrane, four mannose residues are added from Dol-P-Man and three glucose residues from Dol-P-Glc. Dol-P-Man and Dol-P-Glc are also made on the cytoplasmic face of the ER and “flipped” onto the lumenal face. In certain instances, yeast mutants defective in an ALG gene are used to identify the gene that encodes the enzyme responsible for each transfer.

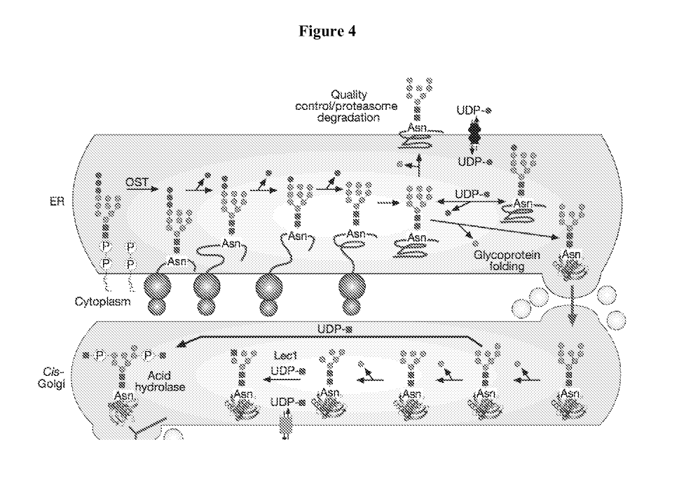

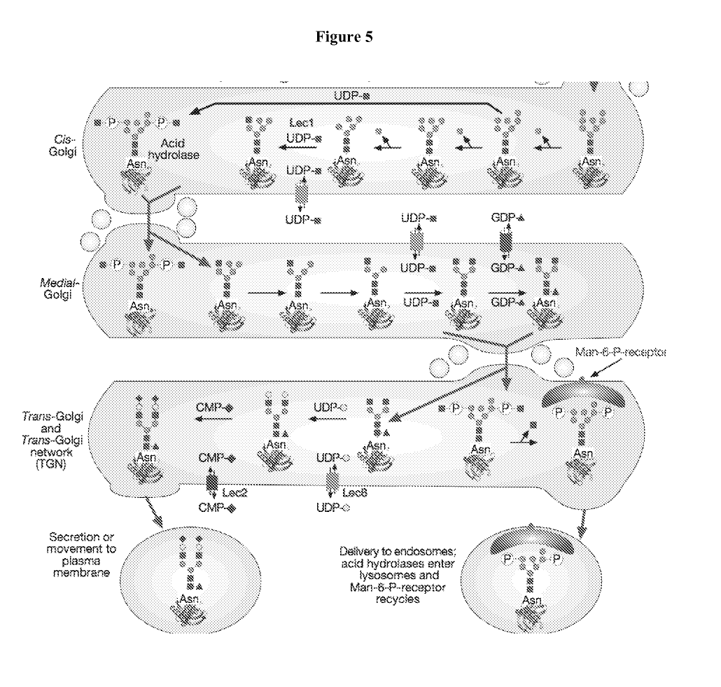

FIGS. 4 and 5 illustrate a processing and maturation of an N-glycan that occurs in certain instances. According to the process illustrated in FIGS. 4 and 5, mature Dol-P-P-glycan, synthesized as described in FIG. 3, is transferred to Asn-X-Ser/Thr sequons during protein synthesis as proteins are being translocated into the ER. Following transfer of the 14-sugar Glc3Man9GlcNAc2 glycan to protein, glucosidases in the ER remove the three glucose residues, and ER mannosidase removes a mannose residue. In some instances, these reactions are intimately associated with the folding of the glycoprotein assisted by the lectins calnexin and calreticulin, and they determine whether the glycoprotein continues to the Golgi or is degraded. In some instances, another lectin, termed EDEM (ER degradation-enhancing α-mannosidase I-like protein), binds to mannose residues on misfolded glycoproteins and escorts them via retrotranslocation into the cytoplasm for degradation. In certain instances, the removal of the first glucose (and therefore all glucose) can be blocked by castanospermine, leaving Glc3Man9GlcNAc2Asn, which may subsequently have terminal mannose residues removed during passage through the Golgi. For most glycoproteins, additional mannose residues are removed in the cis compartment of the Golgi until Man5GlcNAc2Asn is generated. In certain instances, the mannosidase inhibitor deoxymannojirimycin blocks the removal of these mannose residues, leaving Man8GlcNAc2Asn, which is not further processed. In some instances, the action of GlcNAcT-1 on Man5GlcNAc2Asn in the medial-Golgi initiates the first branch of an N-glycan. In certain instances, this reaction is blocked in the Lec1 CHO mutant in which GlcNAcTI is inactive, leaving Man5GlcNAc2Asn, which is not further processed. In some instances, α-Mannosidase II removes two outer mannose residues in a reaction that is blocked by the inhibitor swainsonine. In certain instances, the action of α-mannosidase II generates the substrate for GlcNAcT-II. The biantennary N-glycan resulting according to a process illustrated by FIGS. 4 and 5 is extended by the addition of fucose, galactose, and sialic acid to generate a complex N-glycan with two branches. The addition of galactose does not occur in the Lec8 CHO mutant, which has an inactive UDP-Gal transporter. In Lec8 mutants, complex N-glycans terminate in N-acetylglucosamine. The addition of sialic acid does not occur in the Lec2 CHO mutant, which has an inactive CMP-sialic acid transporter. In Lec2 mutants, complex N-glycans terminate with galactose. Complex N-glycans can have many more sugars than shown in this figure, including additional residues attached to the core, additional branches, branches extended with poly-N-acetyllactosamine units, and different “capping” structures. Also shown is the special case of lysosomal hydrolases that acquire a GlcNAc-1-P at C-6 of mannose residues on oligomannose N-glycans in the cis-Golgi. The N-acetylglucosamine is removed in the trans-Golgi by a glycosidase, thereby exposing Man-6-P residues that are recognized by a Man-6-P receptor and routed to an acidified, prelysosomal compartment.

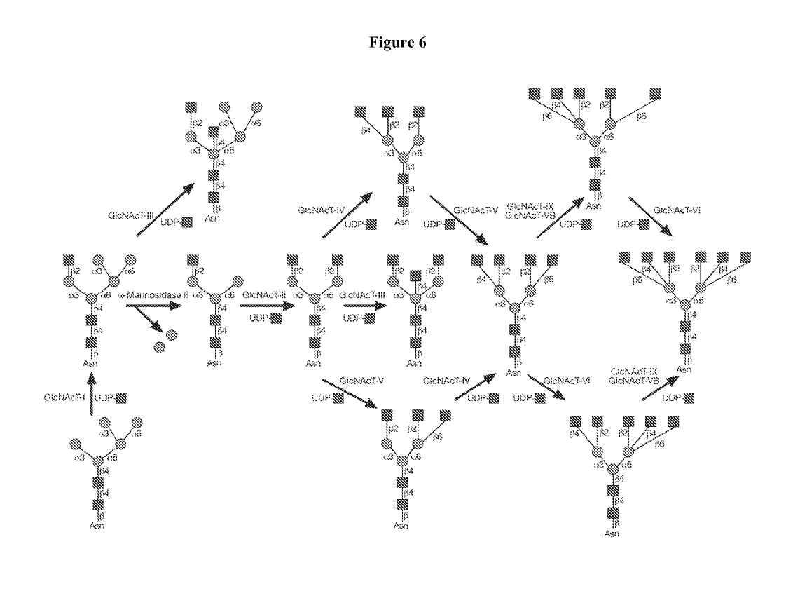

FIG. 6 illustrates the branching of complex N-glycans. Hybrid and mature, biantennary, complex N-glycans may contain more branches due to the action of branching N-acetylglucosaminyltransferases in the Golgi. The latter can act only after the prior action of GlcNAcT-I. GlcNAcT-III transfers N-acetylglucosamine to the β-linked mannose in the core to generate the bisecting N-acetylglucosamine. The presence of this residue may inhibit the action of α-mannosidase II, thereby generating hybrid structures. A biantennary N-glycan may also accept the bisecting N-acetylglucosamine. More highly branched N-glycans can be generated by the action of GlcNAcT-IV, GlcNAcT-V, and GlcNAcT-VI and may also carry the bisecting N-acetylglucosamine. Animals (e.g., mammals and birds) have the potential for generating complex structures. Each N-acetylglucosamine branch may be elongated with galactose, poly-N-acetyllactosamine, sialic acid, and fucose. The bisecting N-acetylglucosamine is not further elongated unless the branch initiated by GlcNAcT-II is missing.

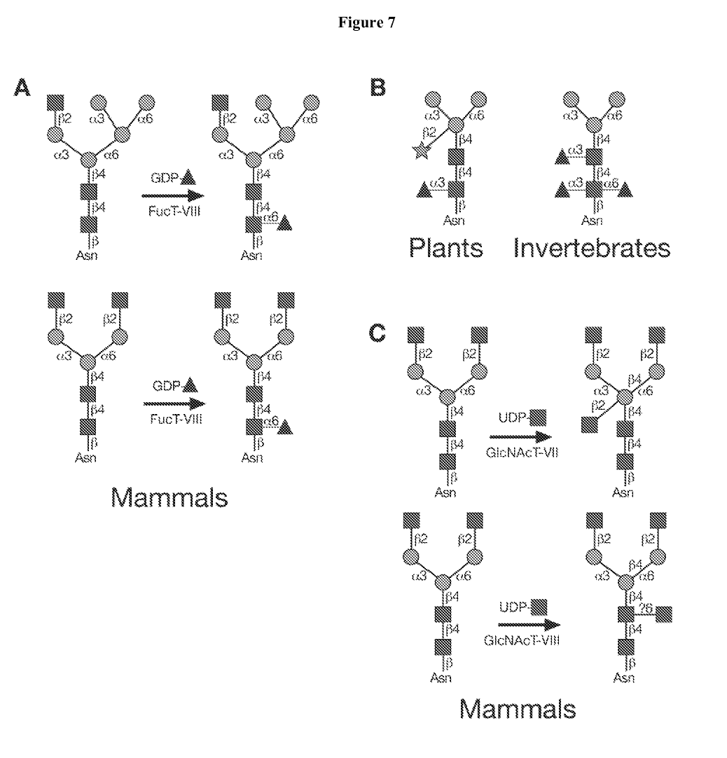

FIG. 7 shows modifications of the core of N-glycans. FIG. 7A illustrates that a fucose residue may be transferred to the core of N-glycans after GlcNAcT-I has acted. Thus, hybrid and biantennary, complex N-glycans may have a core fucose. FIG. 7B illustrates that plants and invertebrates may have additional modifications to the core, with fucose on either N-acetylglucosamine residue (or both for invertebrates) and a xylose attached to the core β-linked mannose residue of plant N-glycans. FIG. 7C illustrates other possible additions to the core in mammalian cells.

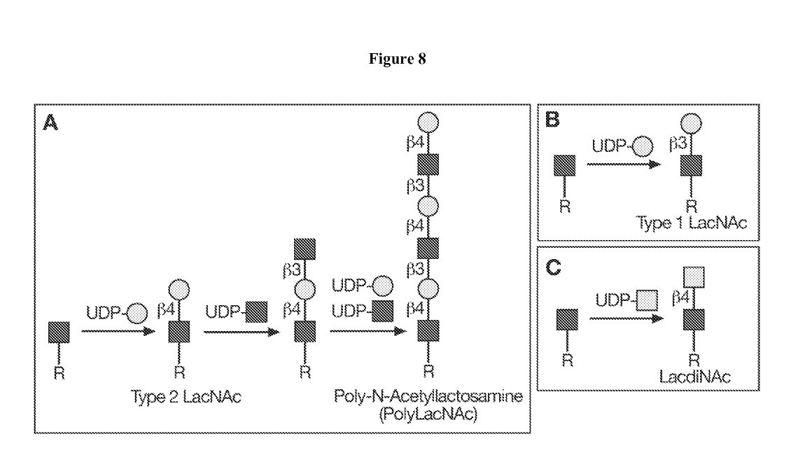

FIG. 8 shows elongation of branch N-acetylglucosamine residues of N-glycans. FIG. 8A illustrates a single N-acetyllactosamine unit that may be generated when galactose is transferred to a branch N-acetylglucosamine on an N-glycan. Further elongation to form poly-N-acetyllactosamine may occur by sequential addition of galactose and N-acetylglucosamine, as shown. This structure is composed of type-2 poly-N-acetyllactosamine units. FIG. 8B illustrates that type-1 N-acetyllactosamine units can also be present in poly-N-acetyllactosamine. FIG. 8C illustrates that transfer of Nacetylgalactosamine to N-acetylglucosamine may generate LacdiNAc.

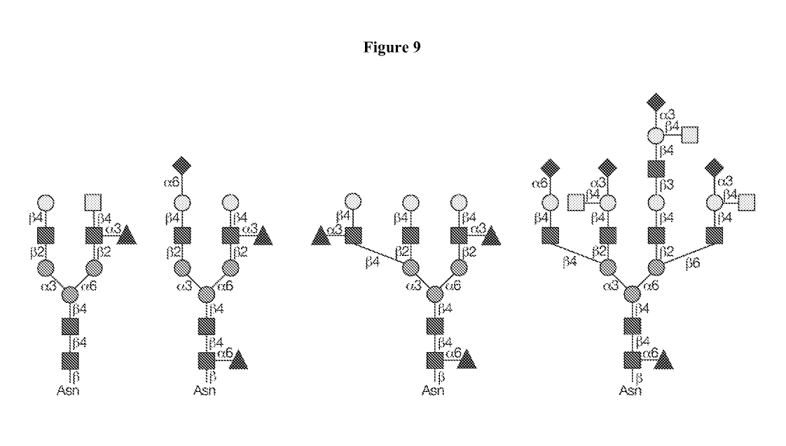

FIG. 9 shows exemplary complex N-glycan structures found on mature glycoproteins.

In some instances, N-linked glycan synthesis inhibitors described herein modulate N-linked glycan biosynthesis, e.g., initiation of the synthesis of a precursor unit, synthesis of a precursor unit, attachment of one or more precursor units to one or more Asn residues in a core protein, further processing (e.g. cleavage of residues) of the Asn-linked precursor unit by glucosidases, synthesis of a Asn-linked pentasaccharide core, further modification of a pentasaccharide core (e.g., polymerization, sialylation, fucosylation, phosphorylation, sulfation, acetylation, galactosylation). In some instances, N-linked glycan synthesis inhibitors described herein modulate chaperones or transporters that mediate glycan biosynthesis.

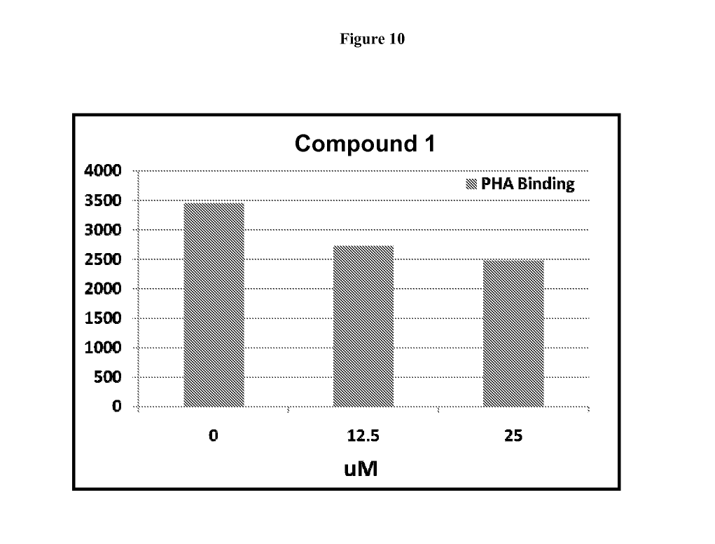

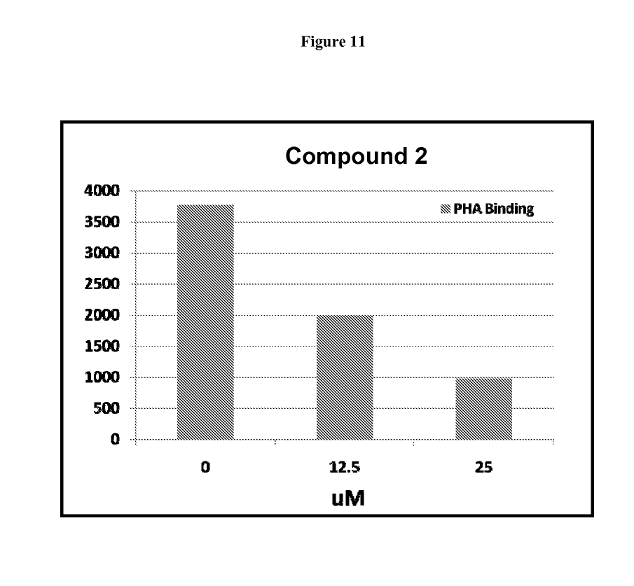

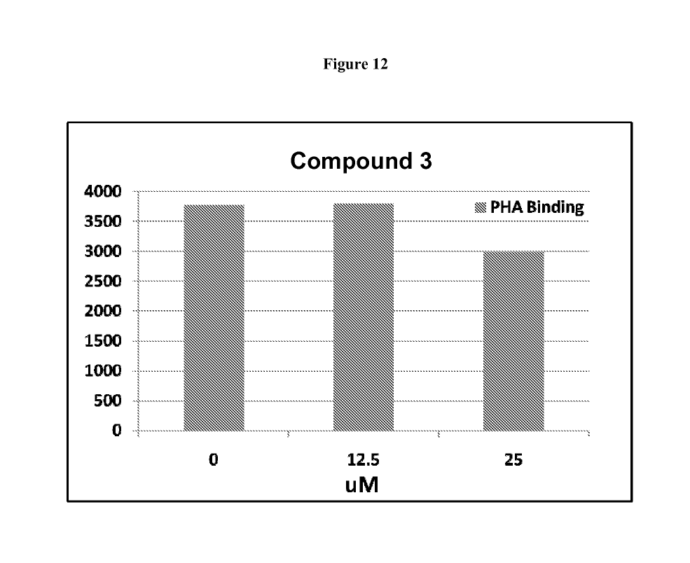

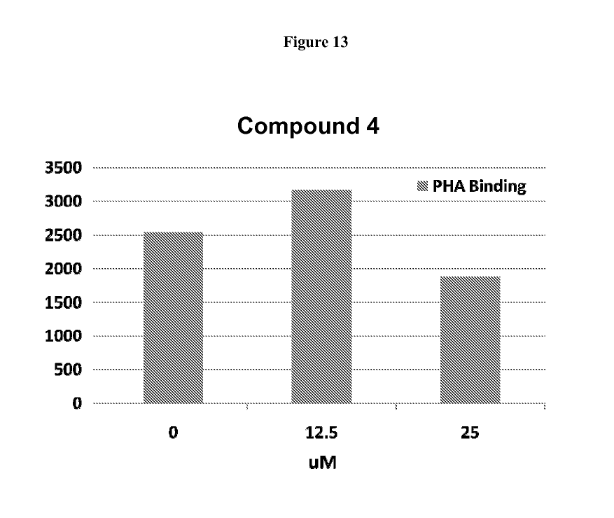

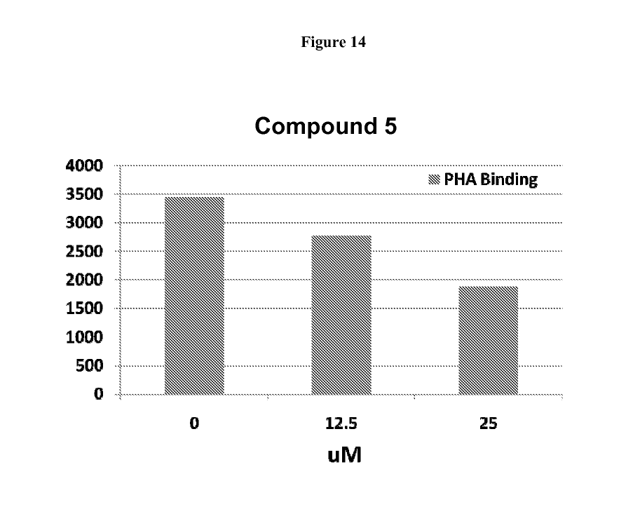

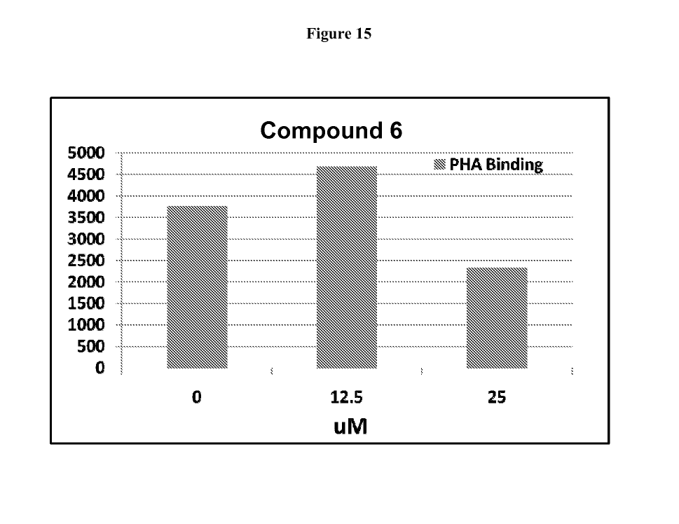

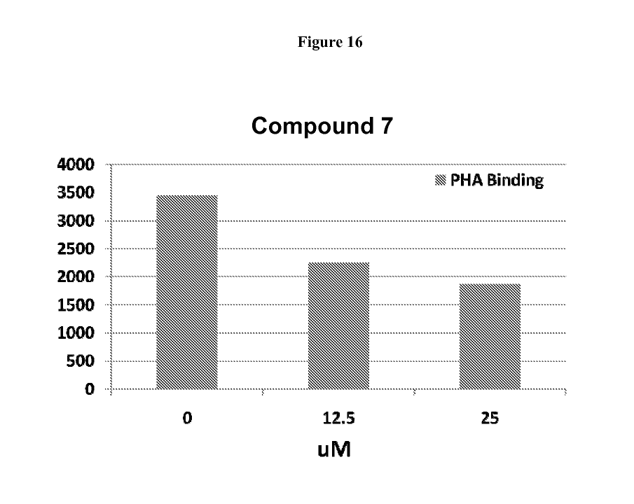

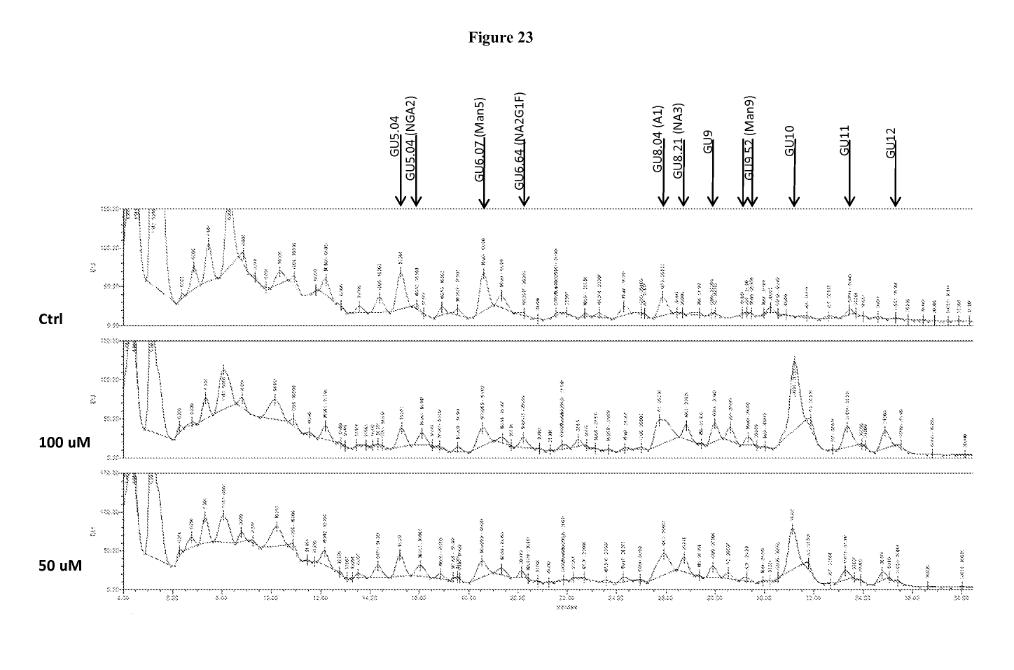

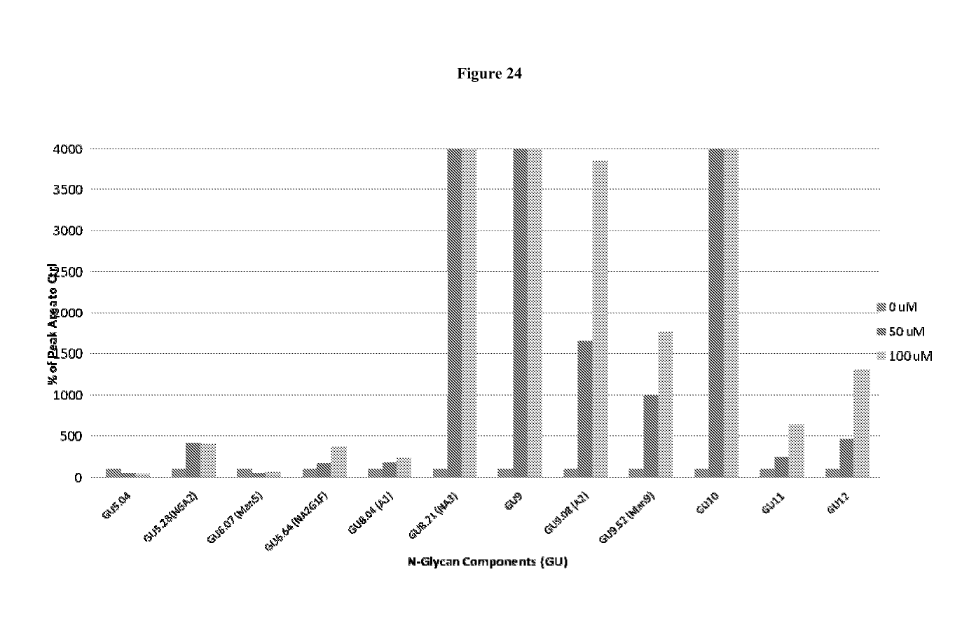

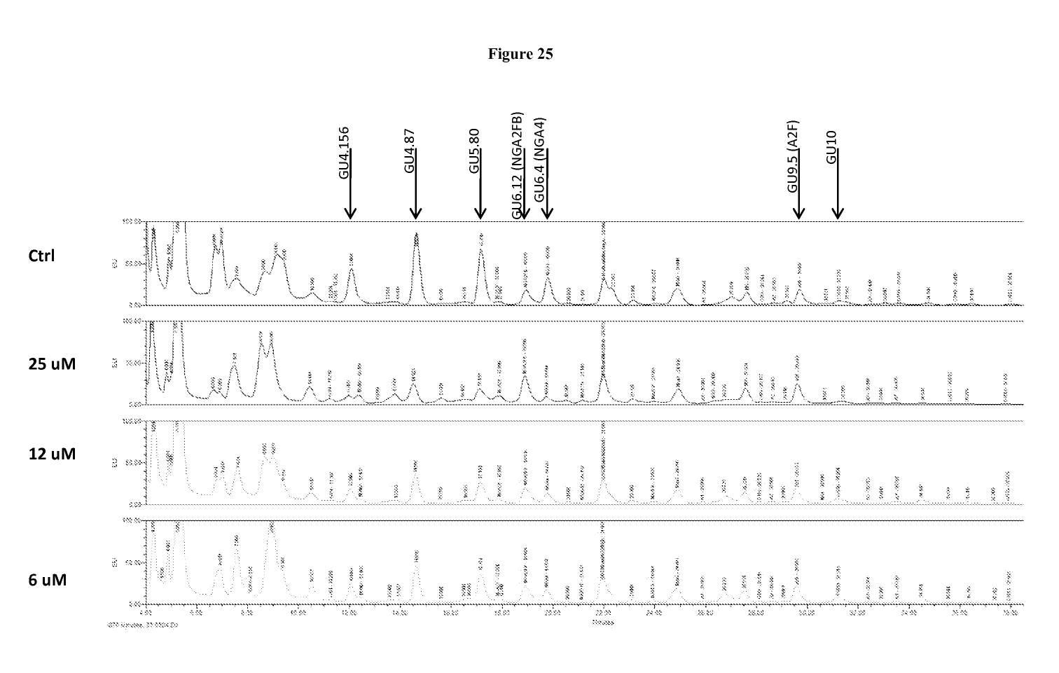

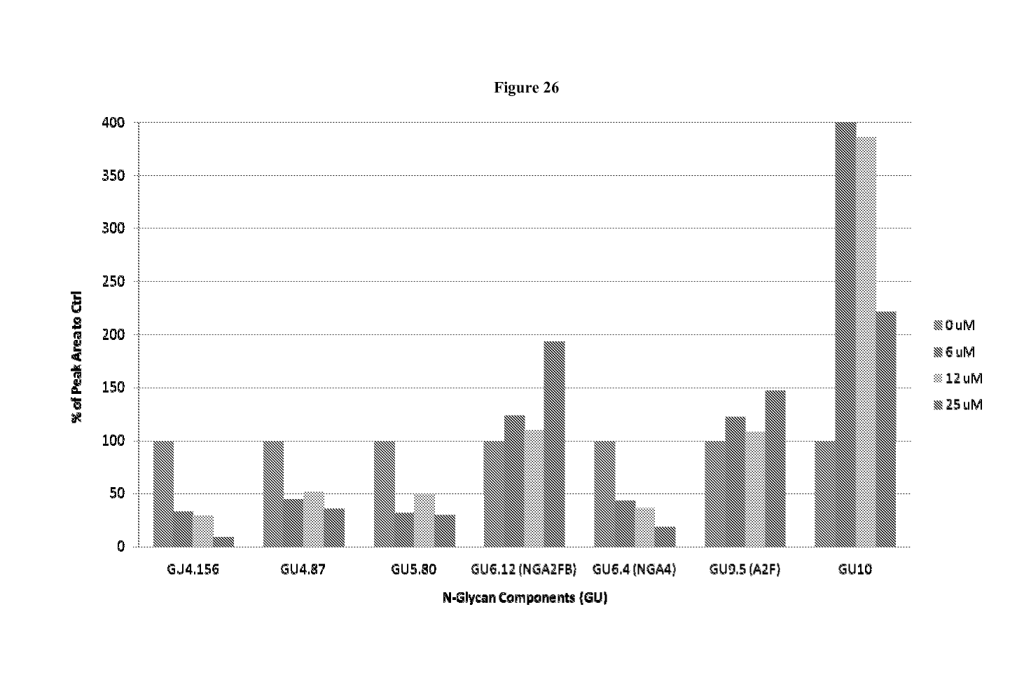

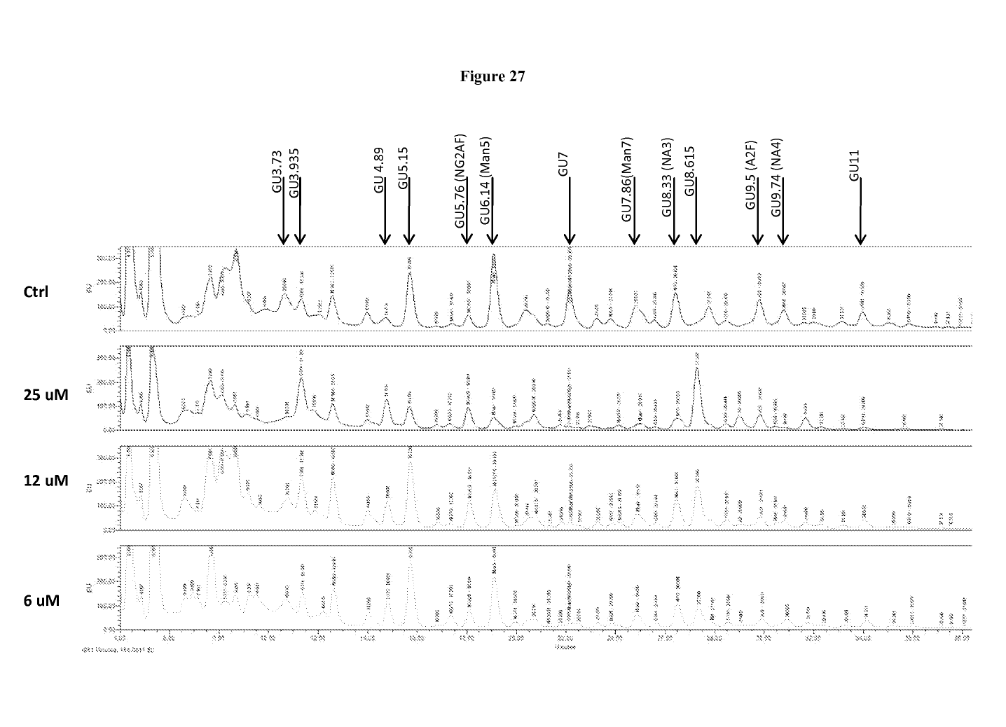

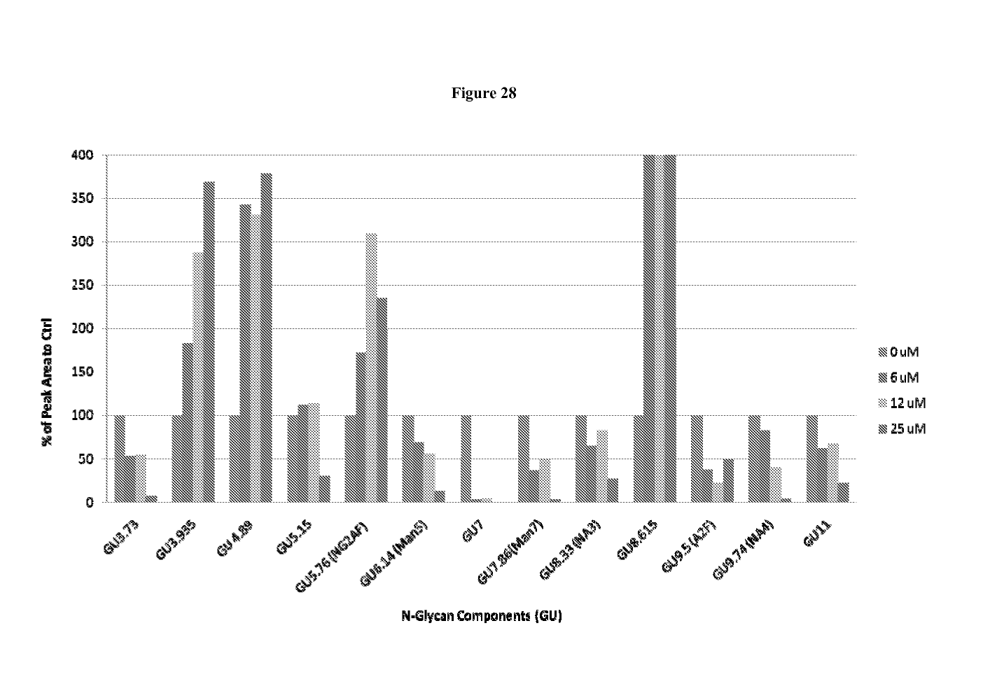

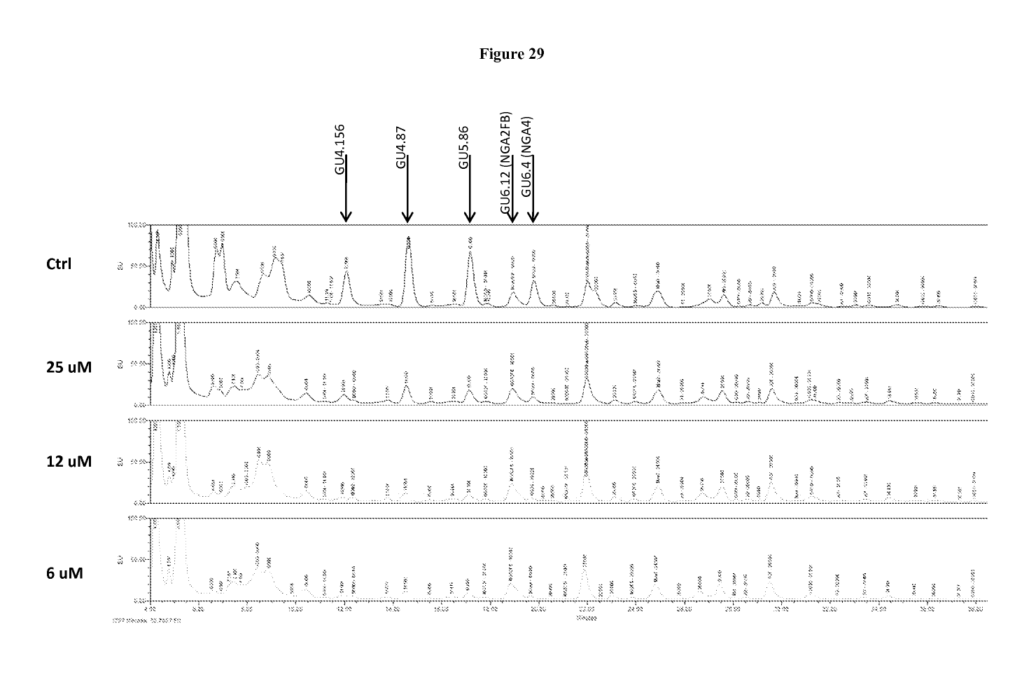

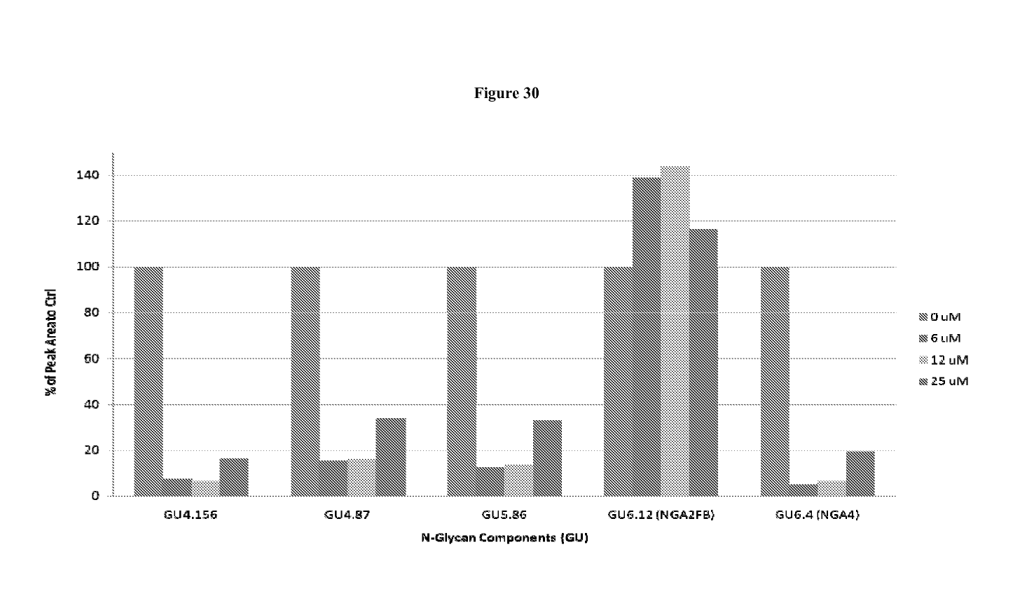

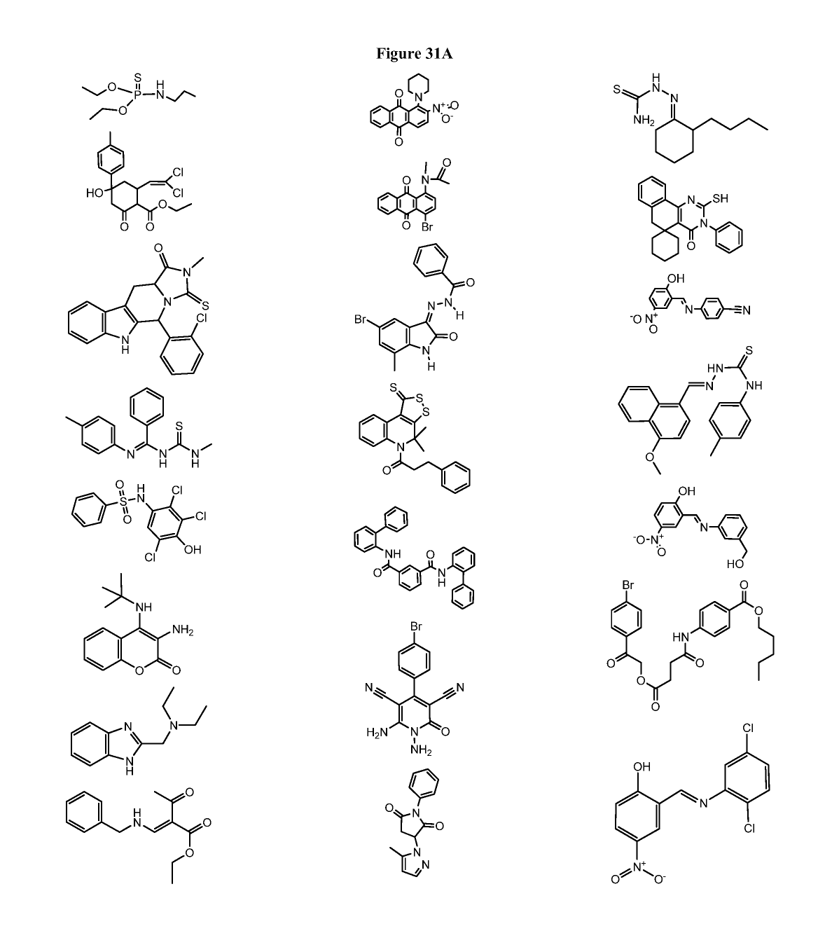

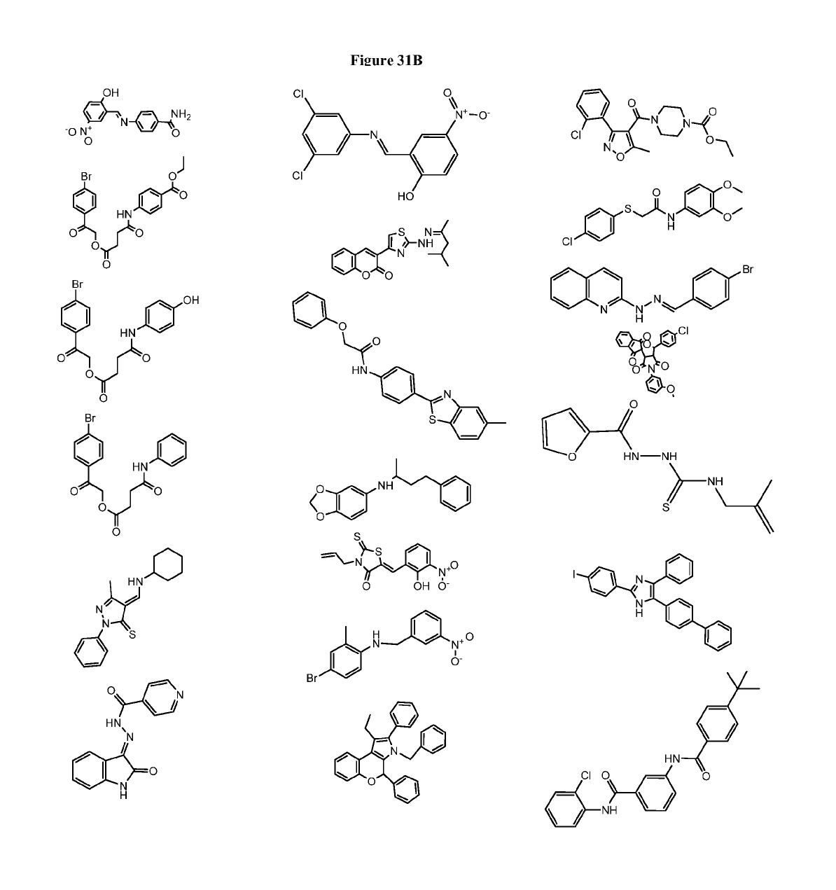

For example, FIGS. 10-16 illustrate that in some embodiments, N-linked glycan biosynthesis inhibitors described herein demonstrate inhibition of the biosynthesis of N-linked glycans with a β1,6 linked GlcNAc branch. Moreover, FIGS. 23-30 demonstrate that N-linked glycans biosynthesis inhibitors described herein alter N-linked glycan structure. FIGS. 23-30 also demonstrate that N-linked glycans biosynthesis inhibitors described herein inhibit steps in the branching and modification phase of N-linked synthesis. This is indicated by specific N-linked structures being changed by the compounds and not elimination of all complex structures with accumulation of high mannose structures (as seen by compounds that inhibit early—Phase I or II—phases of N-linked synthesis.

In some instances, modulation of N-linked glycan biosynthesis includes modulation of the production of the precursor unit (e.g., a (Glcα)₃-(Man α/β)₉-(GlcNAcβ)₂-DolP unit), one or more of the Man residues being optionally phosphorylated. In some embodiments, the modulation of the production of the precursor unit includes the promotion and/or inhibition of the initiation of the synthesis of the precursor unit. In some embodiments, the promotion and/or inhibition of the initiation of the synthesis of the precursor unit of an N-linked glycan includes the promotion and/or inhibition of a UDP-GlcNAc transferase. In some instances, the modulation of the production of the precursor unit includes the promotion and/or inhibition of the synthesis of a precursor unit (e.g., by the promotion and/or inhibition of a GDP mannosyl transferase). In some instances, the modulation of the production of the precursor unit includes the promotion and/or inhibition of the attachment of the precursor unit to an Asn residue on a core protein (e.g., the promotion and/or inhibition of an oligosaccharyl transferase e.g, D-OST).

In some instances, modulation of N-linked glycan biosynthesis includes modulation of further processing of the precursor unit after attachment to an Asn residue of a core protein. In some instances, modulation of further processing of the precursor unit after attachment to an Asn residue of a core protein includes modulation of the synthesis of an N-linked pentasaccharide core. In some instances, modulation of further processing of the precursor unit after attachment to an Asn residue of a core protein and/or modulation of the synthesis of the N-linked pentasaccharide core includes the promotion and/or inhibition of the cleavage of a terminal glucosyl residue in the 14-residue precursor unit. (e.g., the promotion and/or inhibition of α1,2-glucosidase I). In some instances, modulation of further processing of the precursor unit after attachment to an Asn residue of a core protein and/or modulation of the synthesis of the N-linked pentasaccharide core includes the promotion and/or inhibition of cleavage of the remaining glucosyl residues, e.g. the promotion and/or inhibition of α1,3-glucosidase II). In some instances, modulation of further processing of the precursor unit after attachment to an Asn residue of a core protein and/or modulation of the synthesis of the N-linked pentasaccharide core includes the promotion and/or inhibition of cleavage of mannose residues (e.g. a mannose on a (α-1,6) branch), e.g., the promotion and/or inhibition of a mannosidase such as α1,2-manosidase, Golgi mannosidase I (α-1,2 specific), Golgi α-mannosidase II or the like. In some instances, modulation of further processing of the precursor unit after attachment to an Asn residue of a core protein and/or modulation of the synthesis of the N-linked pentasaccharide core includes the promotion and/or inhibition of phosphotransferases e.g., glycophosphorylation by N-acetylglucosaminyl-phosphotransferase and/or removal of GlcNAc by action of N-acetylglucosamine-1-phosphodiester alpha-N-acetylglucosaminidase. In some instances, modulation of further processing of the precursor unit after attachment to an Asn residue of a core protein and/or modulation of the synthesis of the N-linked pentasaccharide core includes the promotion and/or inhibition of receptors, e.g, receptors for (Man α/β)₆-(GlcNAcβ)₂-Asn, one or more of the mannose residues (Man) being optionally phosphorylated, that mediate transport to lysosomes.

In some instances, modulation of N-linked glycan biosynthesis includes modulation of further processing of the pentasaccharide core. In some instances, modulation of further processing of the pentasaccharide core includes modulation of mannosylation of the pentasaccharide core by a mannosyl transferase (e.g. α-1,2 mannosyl transferase). In some instances, modulation of further processing of the pentasaccharide core includes modulation of linkage of a GlcNAc residue β-1,2 to the terminal Man residue of the Man(α1,3) branch and/or the Man(α1,6) branch of the pentasaccharide core by a transferase (e.g., a N-acetylglucosaminyl-transferase I (GlcNAc-T1). In some instances, modulation of further processing of the pentasaccharide core includes modulation of linkage of a bisecting GlcNAc residue (e.g., β1,4GlcNAc) by a transferase (e.g., N-acetylglucosaminyl-transferase III).

In some instances, modulation of N-linked glycan biosynthesis includes modulation of fucosylation, (e.g., α1,6, α1,3) to an N-linked glycan by a fucosyltransferase (e.g., α-1,6 fucosyltransferase, Fuc-TIV, Fuc-TVII or isoforms thereof). In some instances, modulation of N-linked glycan biosynthesis includes modulation of sialylation (e.g., α2,6 or α2,3 to a Gal residue) by a sialyltransferase (e.g., α2,3 sialyltransferase (e.g., ST3Gal IV, ST3Gal VI)). In some instances, modulation of N-linked glycan biosynthesis includes modulation of sialylation e.g., a sialic acid residue is linked α2,8 to another preceding sialic acid. In some instances, modulation of N-linked glycan biosynthesis includes modulation of further polymerization, e.g., linkage of polylactosamine oligosaccharide chains to N-linked glycans (e.g. to Manα-1,6 or to Manα-1,3 of a pentasaccharide core) by e.g., i-extension enzymes (i-GnT), β-1,3 N-acetylglucosaminyltransferases or β1-galactosyltransferases (e.g. (β4Gal-TIV). In some instances, modulation of N-linked glycan biosynthesis includes modulation of glycophosphorylation by N-acetylglucosaminyl-phosphotransferase and/or removal of NAcGlc by action of N-acetylglucosamine-1-phosphodiester alpha-N-acetylglucosaminidase.

In certain embodiments, the modulation of N-linked glycan biosynthesis includes modulation of degradation of N-linked glycans. In some embodiments, the modulation of degradation of N-linked glycans promotes and/or inhibits recycling of saccharide units used for N-linked glycan biosynthesis. In some embodiments, modulation of degradation of N-linked glycans includes modulation of endoglycosidases and/or exoglycosidases. In some embodiments, modulation of endoglycosidases and/or exoglycosidases includes the promotion and/or inhibition of β-Nacetylhexosaminidase (e.g. promotion and/or inhibition of βGlcNAc and/or βGalNAc), sialidase (e.g. neuraminidase), glycosylasparginase, β-galactosidase, β-glucuronidase, α-galactosidase or Cathepsin A.

In certain embodiments, the modulation of N-linked glycan biosynthesis includes modulation of the biosynthesis of β1,6 branched N-linked glycans by GlcNAc transferases (e.g., GlcNAc-TV). In certain embodiments, the modulation of the biosynthesis of β1,6 branched N-linked glycans includes promotion and/or inhibition of GlcNAc transferases (e.g., GlcNAc-TII, GlcNAc-TIV, GlcNAc-TV).

In some embodiments, modulation of β1,6 branched N-linked glycan synthesis includes promotion and/or inhibition of the biosynthesis of binding domains that mediate biological functions, e.g., lectin binding domain of N-linked glycans. In certain embodiments, N-linked glycan biosynthesis inhibitors or modulators of N-linked glycan biosynthesis are compounds that modify the nature (e.g., character, structure and/or concentration) of N-linked glycans endogenous to a cellular compartment (including vesicles), cell, tissue, organ or individual when contacted or administered to the cell, tissue, organ or individual. It is to be understood that contacting a cell, tissue, or organ is possible via the administration to an individual within whom such cell, tissue or organ resides. In certain instances, N-linked glycan biosynthesis inhibitors or modulators of N-linked glycan biosynthesis modify the character and/or concentration of N-linked glycan in a targeted type of cell, tissue type or organ. In other instances, N-linked glycan synthesis inhibitors or modulators of N-linked glycan biosynthesis modify the character and/or concentration of N-linked glycan in a systemic manner.

In some instances, the modulation of N-linked glycan biosynthesis includes promotion and/or inhibition of one or more of UDP-GlcNAc transferase, GDP mannosyl transferase, oligosaccharyl transferase, glucosidases (e.g., α-1,2-glucosidase I, α-1,3-glucosidase II), mannosidase (e.g., α1,2-manosidase, Golgi mannosidase I (e.g., α-1,2 specific), Golgi α-mannosidase II, mannosidase II (e.g. an α1,3/6 mannosidase), N-acetylglucosaminyl-phosphotransferase, N-acetylglucosamine-1-phosphodiester alpha-N-acetylglucosaminidase, GlcNAc-Transferases (e.g., GlcNAc-TII, GlcNAc-TIV, GlcNAc-TV), N-acetylglucosaminyl-transferase III, a fucosyltransferase (e.g., α-1,6 fucosyltransferase) sialyltransferase (e.g., α-2,3 sialyltransferase (e.g., ST3Gal IV, ST3Gal VI)), i-extension enzymes (i-GnT), β-1,3 N-acetylglucosaminyltransferases, β1-4-galactosyltransferases (e.g. β4Gal-TIV), or glycophosphorylation e.g., by N-acetylglucosaminyl-phosphotransferase and/or removal of GlcNAc by action of N-acetylglucosamine-1-phosphodiester alpha-N-acetylglucosaminidase.

In some instances, a single N-linked glycan biosynthesis inhibitor promotes sialylation while inhibiting GalNAc-Ts. In some instances, a single N-linked glycan biosynthesis inhibitor promotes precursor unit synthesis while inhibiting cleavage of the precursor unit (e.g. by a mannosidase). In certain instances, an N-linked glycan biosynthesis inhibitor specifically modulates, promotes or inhibits one or more α2-3 sialyl transferases. In certain instances, an N-linked glycan biosynthesis inhibitor specifically modulates, promotes or inhibits one or more α-1,3 mannosyl transferases. In certain instances, an N-linked glycan biosynthesis inhibitor specifically modulates, promotes or inhibits one or more mannosidases. In certain instances, an N-linked glycan biosynthesis inhibitor specifically modulates, promotes or inhibits iGnT. In certain instances, an N-linked glycan biosynthesis inhibitor specifically modulates, promotes or inhibits β4-Gal-TIV. In certain instances, an N-linked glycan biosynthesis inhibitor specifically modulates, promotes or inhibits GlcNAc-transferases (e.g., GlcNAc-TII, GlcNAc-TIV, GlcNAc-TV). In certain instances, an N-linked glycan biosynthesis inhibitor specifically modulates, promotes or inhibits an oligosaccharyl transferase. In certain instances, an N-linked glycan biosynthesis inhibitor specifically modulates, promotes or inhibits β-Nacetylhexosaminidase (e.g. promotion and/or inhibition of βGlcNAc and/or βGalNAc), sialidase (e.g. neuraminidase), β-galactosidase, β-glucuronidase, α-galactosidase or Cathepsin A. In certain instances, specificity includes inhibition, modulation or promotion of the indicated type of sialylation, fucosylation, mannosylation saccharide transfer, polymerization, degradation and/or initiation by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of sialylation, fucosylation, phosphorylation, sulfation, acetylation, saccharide transfer, polymerization, degradation and/or initiation.

In certain embodiments, an N-linked glycan synthesis inhibitor (used interchangeably herein with a modulator of N-linked glycan biosynthesis) alters or disrupts the nature (e.g. β1,6 linked N-acetylglucosamine linkages, fucosylation or sialylation) of the N-linked glycan compared to endogenous N-linked glycan in an amount sufficient to alter or disrupt N-linked glycan binding, N-linked glycan signaling, or a combination thereof. In some embodiments, the N-linked glycan synthesis inhibitor alters or disrupts the nature of N-linked glycan in a selected tissue type or organ compared to endogenous N-linked glycan in the selected tissue type or organ. In some embodiments, the selected tissue is, by way of non-limiting example, brain tissue, liver tissue, kidney tissue, intestinal tissue, skin tissue, or the like. In some embodiments, an N-linked glycan synthesis inhibitor as described herein alters or disrupts the nature of N-linked glycan compared to endogenous N-linked glycan by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, the N-linked glycan synthesis inhibitor described herein alters or disrupts the concentration of N-linked glycan compared to endogenous N-linked glycan in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, the N-linked glycan synthesis inhibitor described herein alters or disrupts the fucosylation and/or sialylation of N-linked glycan compared to endogenous N-linked glycan in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, the N-linked glycan synthesis inhibitor described herein alters or disrupts the chain length (or N-linked glycan molecular weight) of an N-linked glycan compared to an endogenous N-linked glycan in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more.

In certain embodiments, a N-linked glycan synthesis inhibitor as described herein modifies, alters or disrupts the amount of N-linked glycans on a cell, tissue, organ or individual compared to amounts of endogenous N-linked glycans in an organism, organ, tissue or cell by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, at least about 50%, at least about 60%, at least about 70% or more. As used herein, endogenous N-linked glycan is described as N-linked glycan present in the absence of treatment or contact with a N-linked glycan synthesis inhibitor.

In some embodiments, a modified, altered or disrupted N-linked glycan contains less than about 20%, less than about 30%, less than about 40%, less than about 50%, less than about 60%, less than about 70% or less than about 80% of one or more of bi-antennary, tri-antennary or tetra-antennary N-linked glycans compared to endogenous glycans. By way of example, in some embodiments, a modified, altered or disrupted N-linked glycan contains less than about 20%, less than about 30%, less than about 40%, less than about 50%, less than about 60%, less than about 70% or less than about 80% of bi-antennary N-linked glycans, or less than about 20%, less than about 30%, less than about 40%, less than about 50%, less than about 60%, less than about 70% or less than about 80% of tri-antennary N-linked glycans, or less than about 20%, less than about 30%, less than about 40%, less than about 50%, less than about 60%, less than about 70% or less than about 80% of tetra-antennary N-linked glycans, or a combination thereof, compared to endogenous glycans.

In certain embodiments, N-linked glycan synthesis inhibitor described herein alters or disrupts, in combination (e.g., the sum of the change in amount, concentration, and/or chain length of β1,6 linked N-acetylglucosamine linkages in an N-linked glycan), the nature of an N-linked glycan compared to endogenous N-linked glycan in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, an N-linked glycan synthesis inhibitor as described herein alters or disrupts β1,6 linked N-acetylglucosamine linkages of an N-linked glycan compared to endogenous N-linked glycan in an organism, organ, tissue or cell by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. As used herein, endogenous N-linked glycan is described as N-linked glycan present in the absence of treatment or contact with an N-linked glycan synthesis inhibitor. In some embodiments, the comparison between altered or disrupted N-linked glycan compared to endogenous N-linked glycan is based on the average characteristic (e.g., the concentration, β1,6 linked N-acetylglucosamine linkages, mannosylation, sialylation, chain length or molecular weight, combinations thereof, or the like) of the altered or disrupted N-linked glycan. Furthermore, in some embodiments, the comparison between altered or disrupted N-linked glycan is based on a comparison of the β1,6 linked N-acetylglucosamine linkages of the modified N-linked glycan to the β1,6 linked N-acetylglucosamine linkages of endogenous N-linked glycan. In some instances, the degree or nature of β1,6 linked N-acetylglucosamine linkages increased or decreased in the modified N-linked glycan. Similarly, in certain instances, the degree or nature of β1,6 linked N-acetylglucosamine linkages in the domains that have low β1,6 linked N-acetylglucosamine linkages in endogenous N-linked glycan have β1,6 linked N-acetylglucosamine linkages in the modified N-linked glycan. In some instances, domain organization is determined using enzymes that cleave only β1,6 linked N-acetylglucosamine linkages (e.g., GlcNAc-TV). The concentration, amount, character, and/or structure of an N-linked glycan is determined in any suitable manner, including those set forth herein. As used herein, altering includes increasing or decreasing. Furthermore, as used herein, disrupting includes reducing or inhibiting.

In some embodiments, an N-linked glycan biosynthesis inhibitor alters or disrupts the nature of an N-linked glycan such that it alters or disrupts β1,6 linked N-acetylglucosamine linkages of the N-linked glycan. In some embodiments, the N-linked glycan synthesis inhibitor described herein alters or disrupts the nature of the N-linked glycan such that it inhibits N-linked glycan binding. In some embodiments, the N-linked glycan synthesis inhibitor described herein alters or disrupts the nature of the N-linked glycan such that it inhibits N-linked glycan binding and signaling. In some instances, the N-linked glycan synthesis inhibitor alters or disrupts the nature of the N-linked glycan such that it inhibits the binding, signaling, or a combination thereof of any lectin (including polypeptides) subject to N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments, the CAM is an exogenous CAM, e.g., a bacterial lectin. In certain embodiments, the CAM is and endogenous CAM and includes, by way of non-limiting examples, E-selectin, L-selectin P-selectin, galectin-3, or any one or more galectin. In some instances, the N-linked glycan synthesis inhibitor alters or disrupts the nature of the N-linked glycan such that it inhibits the binding, signaling, or a combination thereof of integrins, matriptase and/or N-cadherin.

In some embodiments, an N-linked glycan biosynthesis inhibitor is an agent that when contacted or administered to a human liver cell, a human liver tissue, a human liver, or a human results in an average number of β1,6 linked N-acetylglucosamine linkages of less than about 1.2, less than about 1.1, less than about 1.0, less than about 0.9, less than about 0.8, less than about 0.7, less than about 0.6, or less than about 0.5 in the liver cell, liver tissue, the liver, or the liver of the human, respectively. As used herein, the average number of β1,6 linked N-acetylglucosamine linkages refers to the number of β1,6 linked N-acetylglucosamine linkages on each N-linked glycan chain (e.g., on each high mannose, hybrid or complex N-linked glycan chain). In some embodiments, an N-linked glycan biosynthesis inhibitor is an agent that when contacted or administered to a pig liver cell, pig liver tissue, a pig liver, or a pig results in an average number of β1,6 linked N-acetylglucosamine linkages of less than about 1.0, less than about 0.9, less than about 0.8, less than about 0.7, less than about 0.6, or less than about 0.5 in the liver cell, liver tissue, the liver, or the liver of the pig, respectively. In some embodiments, an N-linked glycan biosynthesis inhibitor is an agent that when contacted or administered to a mouse liver cell, mouse liver tissue, a mouse liver, or a mouse results in an average number of β1,6 linked N-acetylglucosamine linkages of less than about 0.9, less than about 0.8, less than about 0.7, less than about 0.6, less than about 0.5, less than about 0.4, or less than about 0.3 in the liver cell, liver tissue, the liver, or the liver of the mouse, respectively.

In some embodiments, an N-linked glycan biosynthesis inhibitor is an agent that when contacted or administered to a human liver cell, a human liver tissue, a human liver, or a human results in an average β1,6 linked N-acetylglucosamine residues of less than about 1.2 mol. %, less than about 1.1 mol. %, less than about 1.0 mol. %, less than about 0.9 mol. %, less than about 0.8 mol. %, less than about 0.7 mol. %, less than about 0.6 mol. %, or less than about 0.5 mol. % in the liver cell, liver tissue, the liver, or the liver of the human, respectively. As used herein, mol. % is the molar percentage of the selected saccharide component compared to the total number of saccharide components in the N-linked glycan present and/or analyzed. In some embodiments, an N-linked glycan biosynthesis inhibitor is an agent that when contacted or administered to a human liver cell, a human liver tissue, a human liver, or a human results in average β1,6 linked N-acetylglucosamine residues of less than about 15 mol. %, less than about 14 mol. %, less than about 12 mol. %, less than about 10 mol. %, less than about 8 mol. %, less than about 7 mol. %, less than about 6 mol. %, or less than about 5 mol. % in the liver cell, liver tissue, the liver, or the liver of the human, respectively. In some embodiments, an N-linked glycan biosynthesis inhibitor is an agent that when contacted or administered to a human liver cell, a human liver tissue, a human liver, or a human results in average β1,6 linked N-acetylglucosamine residues of less than about 7 mol. %, less than about 6 mol. %, less than about 5 mol. %, less than about 4 mol. %, less than about 3 mol. % in the liver cell, liver tissue, the liver, or the liver of the human, respectively. In some embodiments, an N-linked glycan biosynthesis inhibitor is an agent that when contacted or administered to a human liver cell, a human liver tissue, a human liver, or a human results in average β1,6 linked N-acetylglucosamine residues of less than about 0.7 mol. %, less than about 0.6 mol. %, less than about 0.5 mol. %, less than about 0.4 mol. %, or less than about 0.3 mol. % in the liver cell, liver tissue, the liver, or the liver of the human, respectively.

In certain embodiments, the amount of an N-linked glycan synthesis inhibitor administered is an effective amount. In further embodiments, the effective amount is an amount having a minimal lethality. In more specific embodiments, the LD₅₀:ED₅₀ is greater than about 1.1, greater than about 1.2, greater than about 1.3, greater than about 1.4, greater than about 1.5, greater than about 2, greater than about 5, greater than about 10, or more. In some embodiments, a therapeutically effective amount is about 0.1 mg to about 10 g.

### Selectivity

In some embodiments, a N-linked glycan biosynthesis inhibitor described herein is a selective N-linked glycan synthesis inhibitor. In some embodiments, a selective N-linked glycan inhibitor selectively alters or disrupts the nature (e.g., concentration, chain length, average number of sialic acid residues, bi-antennary or tri-antennary or tetra-antennary N-linked glycans etc.) of an N-linked glycan. In certain instances, limiting modifications to glycans limits undesirable or toxic side effects. In some instances, further restrictions to subsets of glycans, further restrict side effects and makes identification, isolation and tracking the effects of the inhibitors more reliable. In some instances, this makes dose determination more reliable.

In certain embodiments, N-linked glycan biosynthesis inhibitors include inhibitors that are selective for a glycan (carbohydrate portion of a molecule) not protein, not nucleic acid, not lipid. In some embodiments, N-linked glycan biosynthesis inhibitors include inhibitors that are selective for specific glycans and/or specific glycans linked to an asparagine residue (Asn) on a protein, such as:
a. Glycans containing glucose (Glu)
b. Glycans containing galactose (Gal)
c. Glycans containing N-acetylglucosamine (GlcNAc)
d. Glycans containing N-acetylgalatosamine (GalNAc)
e. Glycans containing mannose (Man)
f. Glycans containing xylose (Xyl)
g. Glycans containing fucose (Fuc)
h. Glycans containing sialic acid (Sia)
i. Glycans containing GlcNAc and Man
j. Glycans with the structure Asn-βGlcNAc(β1-4)GlcNAc(β1-4)Man[(α1-3)Man(β1-2)GlcNAc](α1-6)Man(β1-2)GlcNAc
k. Glycans with the structure Asn-βGlcNAc[(α1-6)Fuc](β1-4)GlcNAc(β1-4)Man[(α1-3)Man(β1-)GlcNAc](α1-6)Man(β1-2)GlcNAc
l. Glycans with the structure Asn-βGlcNAc(β1-4)GlcNAc(β1-4)Man[(α1-3)Man(β1-2)GlcNAc](α1-6)Man[(β1-2)GlcNAc](β1-6)GlcNAc
m. Glycans with the structure Asn-βGlcNAc(β1-4)GlcNAc(β1-4)Man[(α1-3)Man[[(β1-2)GlcNAc](β1-4)GlcNAc](α1-6)Man[(β1-2)GlcNA](β1-6)GlcNAc
n. More highly branched structures can be generated from structures x, xi, xii and xiii by the actions of GlcNAcT-IV, GlcNAcT-IV, GlcNAcT-IV, GlcNAcT-IV and GlcNAcT-IV: e.g. Asn-βGlcNAc(β1-4)GlcNAc(β1-4)Man[(α1-3)Man[[(β1-2)GlcNA4(β1-4)GlcNAc](α1-6)Man[[(β1-2)GlcNAc][(β1-4)GlcNAc]] (β1-6)GlcNAc and Asn-βGlcNAc(β1-4)GlcNAc(β1-4)Man[(α1-3)Man[[(β1-2)GlcNAc][(β1-4)GlcNAc][(β1-6)GlcNAc]] (α1-6)Man[[(β1-2)GlcNAc][([1-4)GlcNAc]](β1-6)GlcNAc
o. Glycans with N-acetylglucosmine branches elongated with galactose, poly-N-acetyllactosamine, sialic acid and fucose
p. Glycans of j, k, l, m, or n with one or more galactose residues bound to the terminal GlcNAc residues
q. Glycans of j, k, l, m, or n with one or more GalNAc residues bound to the terminal GlcNAc residues
r. Glycans of j, k, l, m, or n with one or more fucose esidues bound to the terminal GlcNAc residues
s. Glycans of j, k, l, m, or n with additional saccharide structures comprised of a combination of none, one or more Gal, GalNAc, GlcNAc, Fuc and Sia residues bound to the one or more of the four terminal GlcNAc residues
t. Glycans of j, k, l, m, or n with additional saccharide structures bound (α1-6) to the (α1-6)bound Man residue containing serial disaccharides of GlcNAc(β1-4)Gal bound together by Gal(β1-3)GlcNAc linkages forming chains called poly-N-acetylyllactosamine chains.
u. Glycans of j, k, l, m, or n with additional saccharide structures bound (α1-6) to the (α1-6)bound Man residue containing serial disaccharides of GlcNAc(β1-4)Gal bound together by Gal(β1-3)GlcNAc linkages forming chains called poly-N-acetylyllactosamine chains. These poly-N-acetylyllactosamine chains further acted upon by β1-6 N-acetyglucosamine transferases to transfer β1-6 linked N-acetylglucosamine residues to internal Gal residues to form “I” and “I” blood group antigens
v. Glycans of j, k, l, m, or n with additional saccharide structures bound (α1-6) to the (α1-6)bound Man residue containing serial disaccharides of GlcNAc(β1-4)Gal bound together by Gal(β1-3)GlcNAc linkages forming chains called poly-N-acetylyllactosamine chains. These poly-N-acetylyllactosamine chains further acted upon by glycosyltransferases to from structures containing Gal, GlcNAc, GalNAc and Fuc to form the A, B, and H blood group antigens.
w. Glycans of j, k, l, m, or n with additional saccharide structures bound (α1-6) to the (α1-6)bound Man residue containing serial disaccharides of GlcNAc(β1-4)Gal bound together by Gal(β1-3)GlcNAc linkages forming chains called poly-N-acetylyllactosamine chains. These poly-N-acetylyllactosamine chains further acted upon by glycosyltransferases and sulfotransferases to from structures containing Gal, GlcNAc, GalNAc, Fuc, Sia, sulfated Gal and sulfated GlcNAc to form the Lewis blood group antigens.
x. Glycans and glycolipids acted upon by a specific (α1-3) galactose transferase (α1-3GalT) to form the Gal(α1-3)Gal epitope on the termini of type-2 units in the tissues of New World primates and many nonprimate mammals but absent from Old World primates (Homo sapiens).

In certain embodiments, N-linked glycan biosynthesis inhibitors described herein selectively inhibit the biosynthesis of N- and O-linked glycoproteins and glycolipids containing sialic acid residues α2-3 linked to terminal galactose residues (in vertebrates)—catalyzed by 6 α2-3 sialyltransferases ST3GalI to ST3GalVI. In some embodiments, N-linked glycan biosynthesis inhibitors described herein selectively inhibit the biosynthesis of N- and O-linked glycoproteins and glycolipids containing sialic acid residues α2-6 linked to terminal Gal residues, terminal or subterminal GalNAc residues (ST6Gal-I, ST6Gal-II and ST6GalNAc-I-ST6GalNAc-IV) or on internal GalNAc (ST6GalNAc-III) or α2-6 sialic acid (e.g., in a human suffering from an influenza viral infection). In certain embodiments, N-linked glycan biosynthesis inhibitors described herein selectively inhibit the biosynthesis of N- and O-linked glycoproteins and glycolipids containing fucose.

Moreover, in certain instances, targeting early biosynthetic enzymes eliminates or reduces N-linked glycans which have global effects on protein folding, protein solubility and protein processing. These effects could be extremely toxic or lethal. Thus, in some instances, targeting late enzymes blocks modifications that involve more specific receptor binding that is involved in certain cellular adhesion and trafficking interactions. In certain instances, specific interactions involving late pathway enzymes could be controlled more readily and under controlled conditions (appropriate dosing) and provide beneficial effects for a number of diseases. Thus, in certain embodiments herein an N-linked glycan biosynthesis inhibitor is a selective inhibitor of late phase N-linked glycan biosynthesis (e.g., selectively inhibits any one or more late phase biosynthetic process of N-linked glycan biosynthesis). In some embodiments, late in the biosynthetic pathway refers to structures late in the branching and modification phase (e.g., Phase III) or later (see, e.g., FIGS. 5-6). For example, in some instances, late in the biosynthetic pathway refers to biosynthetic processes (or glycans synthesized thereby) following the removal of 6 mannose residues by α-mannosidase I and α-mannosidase II. In certain instances, glycans late in the biosynthetic pathway includes GlcNAc₂Man₃ and structures that are produced subsequently by further processing in the medial golgi and beyond (see Medial Golgi in FIG. 5). In certain embodiments, a late stage biosynthesis inhibitor described herein is an inhibitor that acts in the N-linked glycan biosynthetic pathway after or downstream from mannosidase II.

In some embodiments, a selective inhibitor of N-linked glycan synthesis selectively reduces or inhibits the synthesis of bi-antennary, tri-antennary or tetra-antennary N-linked glycans compared to other N-linked glycans. In certain embodiments, selective N-linked glycan synthesis inhibitors selectively inhibit synthesis of bi-antennary and/or tri-antennary and/or tetra-antennary N-linked glycans compared to extracellular glycans by a ratio of greater than about 2:1, about 3:1, about 4:1, about 5:1, about 6:1, about 8:1, about 10:1 or more.

In certain embodiments, selectivity of an N-linked glycan synthesis inhibitors is beneficial in order to target specific disorders without adversely impacting properly functioning glycan biosynthetic processes. In some embodiments, therapeutic methods utilizing selective N-linked glycan synthesis inhibitors have improved toxicity profiles compared to non-selective glycan synthesis inhibitors. In some embodiments, selective N-linked glycan synthesis inhibitors modulate (e.g., inhibit or promote) late stage processes (including, e.g., enzyme activity involved in the N-linked glycan preparation/synthetic pathway, enzyme activity involved in the N-linked glycan degradation pathway, other enzyme activity that affects the character of N-linked glycans, or the like) in the N-linked glycan biosynthetic pathway.

In certain embodiments, the selective N-linked glycan synthesis inhibitor selectively affects the biosynthesis of extracellular glycans, such as N-linked, O-linked, lipid linked, or the like, but not glycosaminoglycans (GAGs), such as heparan sulfate, chondroitin sulfate, dermatan sulfate, keratin sulfate, and/or hyaluronan. In certain embodiments, selective N-linked glycan inhibitors selectively inhibit extracellular glycans compared to GAGs by a ratio of greater than 2:1, 3:1, 4:1, 5:1, 6:1, 8:1, 10:1 or more. In some embodiments, the selective N-linked synthesis inhibitor selectively affects/inhibits the biosynthesis of N-linked glycans, but not GAGs, such as heparan sulfate, chondroitin sulfate, dermatan sulfate, keratin sulfate, and/or hyaluronan, gangliosides, or O-linked glycans. In certain embodiments, selective N-linked glycan inhibitors selectively inhibit N-linked glycans compared to GAGs, gangliosides and/or O-linked glycans by a ratio of greater than 2:1, 3:1, 4:1, 5:1, 6:1, 8:1, 10:1 or more.

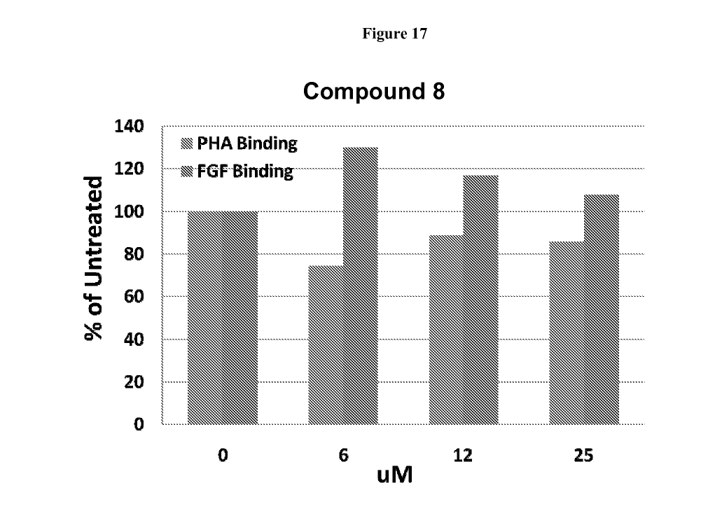

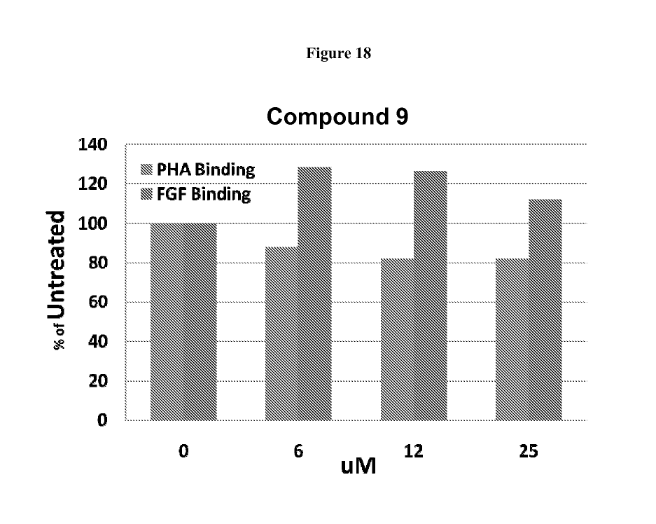

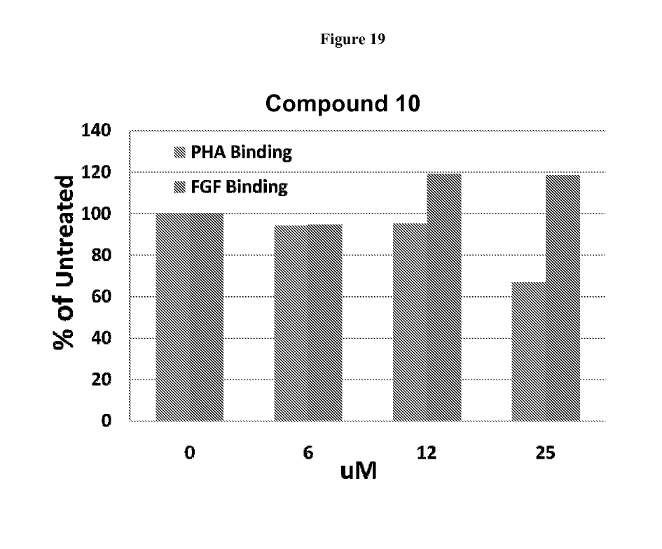

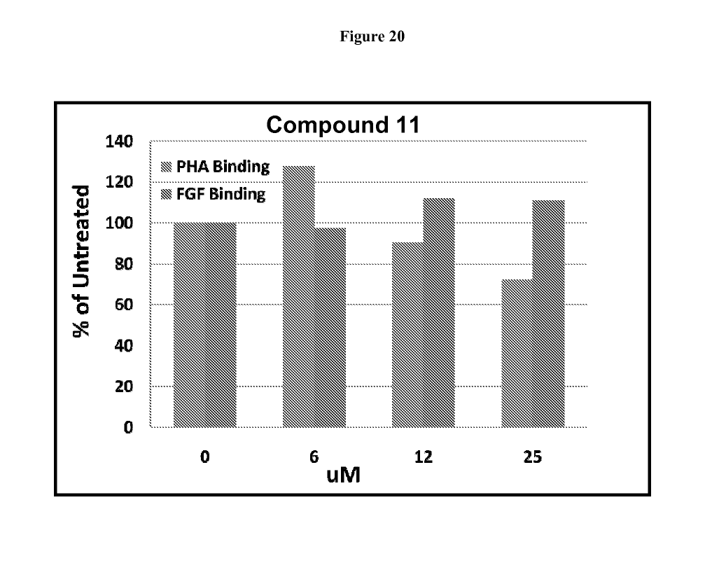

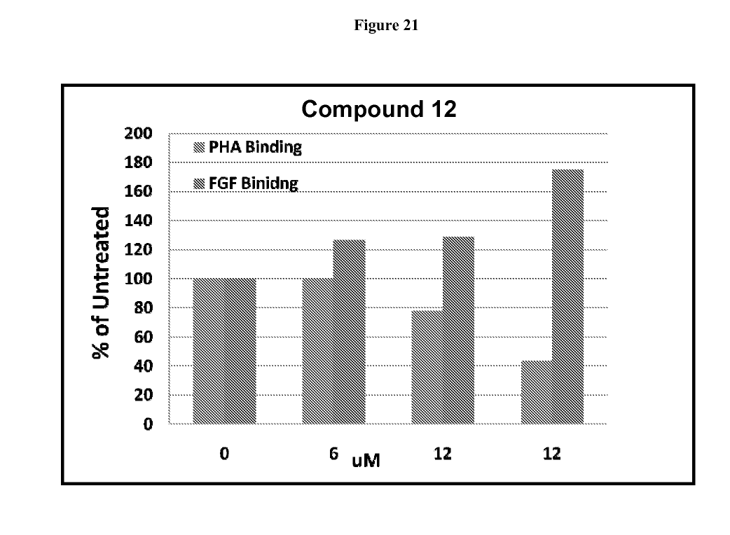

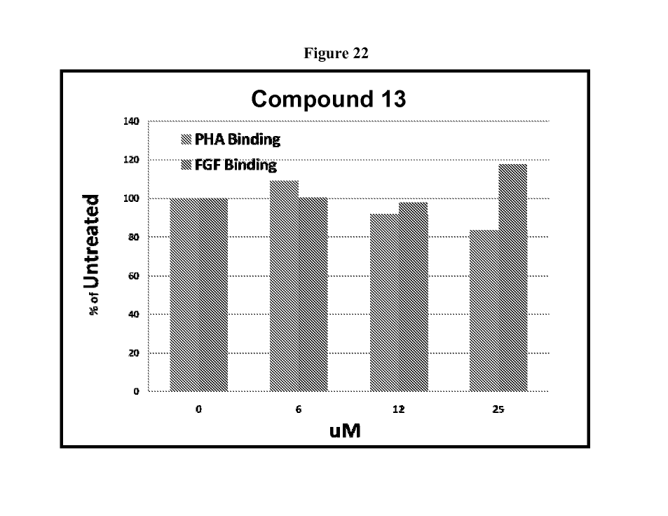

While PHA binding to cells is dependent on N-linked glycans, FGF2 binding is dependent on heparan sulfate. FIGS. 11-22 illustrate that N-linked glycan synthesis inhibitors according to certain embodiments herein show selective inhibition of N-linked glycans without inhibiting other unrelated glycans, such as glycosaminoglycans (e.g., heparan sulfate). Thus, in certain embodiments, compounds described herein have glycan class selectivity

In some embodiments, the selective N-linked synthesis inhibitor selectively affects/inhibits/modulates high mannose N-linked glycan biosynthesis, but does not substantially affect/inhibit/modulate/promote N-linked glycan biosynthesis of the hybrid or complex subtypes. In certain embodiments, selective N-linked glycan inhibitors selectively inhibit N-linked glycans of the high mannose subtype compared to N-linked glycan biosynthesis of the hybrid and/or complex subtypes by a ratio of greater than 2:1, 3:1, 4:1, 5:1, 6:1, 8:1, 10:1 or more. In some embodiments, the selective N-linked synthesis inhibitor selectively affects/inhibits/modulates/promotes N-linked glycan biosynthesis of the hybrid subtype, but does not substantially affect/inhibit/modulate/promote N-linked glycan biosynthesis of the high mannose or complex subtypes. In certain embodiments, selective N-linked glycan inhibitors selectively inhibit N-linked glycans of the hybrid subtype compared to N-linked glycan biosynthesis of the high mannose and/or complex subtypes by a ratio of greater than 2:1, 3:1, 4:1, 5:1, 6:1, 8:1, 10:1 or more. In some embodiments, the selective N-linked synthesis inhibitor selectively affects/inhibits/modulates N-linked glycan biosynthesis of the complex subtype, but does not substantially affect/inhibit/modulate/promote N-linked glycan biosynthesis of the high mannose or hybrid subtypes. In certain embodiments, selective N-linked glycan inhibitors selectively inhibit N-linked glycans of the complex subtype compared to N-linked glycan biosynthesis of the high mannose and/or hybrid subtypes by a ratio of greater than 2:1, 3:1, 4:1, 5:1, 6:1, 8:1, 10:1 or more.

In some embodiments, the selective N-linked synthesis inhibitor selectively affects/inhibits/modulates/promotes one or more enzyme, or the activity thereof, involved in one or more process involved in the early stage biosynthesis of N-linked glycans, but does not significantly affect/inhibit/modulate/promote one or more enzyme, or activity thereof, involved in the middle or late stages of N-linked glycan biosynthesis. In specific embodiments, enzymes selectively affected/inhibited/modulated/promoted by a selective N-linked synthesis inhibitor include, by way of non-limiting example, enzymes involved in the synthesis of the N-linked glycan precursor (e.g., one or more of GlcNAc-1-phosphotransferase, GlcNAc-transferase, mannosyltransferase (e.g., transferases involved in the biosynthesis of the first 5 mannose residues, mannosyltransferase on the cytoplasmic side of the ER, mannosyltransferase involved in the biosynthesis of the final 4 mannose residues on the luminal side of the ER, glucosyltransferase involved in the biosynthesis of the final 3 glucose residues on the luminal side of the ER, or a combination thereof), and/or an oligosaccharyl transferase (OST) enzyme involved in the transfer of dolichol phosphate (Dol-P) to the Asn residue of a protein. In specific embodiments, a selective inhibitor described herein optionally selectively inhibits any one or more of these early enzymes compared to other enzymes of the group, or any other enzyme involved in the biosynthesis of N-linked glycans.

In some embodiments, the selective N-linked synthesis inhibitor selectively affects (e.g., inhibits, modulates, or promotes) one or more process or enzyme, or the activity thereof, involved in one or more process involved in the middle biosynthesis stages of N-linked glycans, but does not significantly affect (e.g., inhibit, modulate, or promote) one or more process or enzyme, or activity thereof, involved in the early or late stages of N-linked glycan biosynthesis. In specific embodiments, enzymes or processes selectively affected (e.g., inhibited, modulated, or promoted) by a selective N-linked synthesis inhibitor include, by way of non-limiting example, enzymes or processes involved in the processing to form the high mannose subtype (e.g., enzymes or processes involved in the removal of the 3 glucose residues, such as glucosidases I and II, calnexin and/or calrecticulin binding, alpha-glucosyl transferase that can regulosylate, or the like; or enzymes or processes involved in the removal of one mannose residue, such as by alpha-mannosidase I); and/or enzymes or processes involved in the processing in the Cis-golgi, such as GlcNAc-phosphotransferases and/or alpha-N-acetylglucosaminidase (e.g., in processes wherein, for proteins transferred to the lysozyme (hydrolases), N-glycans are modified by GlcNAc-phosphotransferase and the GlcNAc residues subsequently removed by GlcNAc-1-phosphodiester alpha-N-acetylglucosaminidase leaving mannose-6-phosphate residues, the proteins being taken up by the lysosomes through binding to mannose-6-phosphate receptors), or class 1 alpha-mannosidases (e.g., as acting on alpha-1,2-linked mannose residues to form Man₅GlcNAc₂-Asn glycan that becomes the substrate in the Golgi diversification of extracellular glycans). In specific embodiments, a selective inhibitor described herein optionally selectively inhibits any one or more of these early enzymes compared to other enzymes of the group, or any other enzyme involved in the biosynthesis of N-linked glycans.

In some embodiments, the selective N-linked synthesis inhibitor selectively affects (e.g., inhibits, modulates, or promotes) one or more process or enzyme, or the activity thereof, involved in the one or more process involved in the late biosynthesis stages of N-linked glycans, but does not significantly affect (e.g., inhibit, modulate, or promote) one or more process or enzyme, or activity thereof, involved in the early or middle stages of N-linked glycan biosynthesis. In specific embodiments, enzymes or processes selectively affected (e.g., inhibited, modulated, or promoted) by a selective N-linked synthesis inhibitor include, by way of non-limiting example, enzymes or processes involved in the biosynthesis (e.g., N-linked glycan diversification) of hybrid N-linked glycans, such as, GlcNAc transferase I, alpha-mannosidase II, GlcNAc transferase II, or a combination thereof. In certain instances, N-linked glycans of the hybrid subtype undergo late stage biosynthesis starting with substituted mannose residues, to which GlcNAcTI adds GlcNac in a beta-1,2-linkage to the alpha 1,3-mannose; in some instances, the resulting hybrid glycan is a specific substrate for alpha-mannosidase II, which removes the alpha 1,3 and alpha 1,6 mannose residues which are linked to the alpha-1,6 branch; in certain instances, the resulting glycan is a substrate for GlcNAcTII, which converts hybrid N glycans to complex N-glycans by adding GlcNAc to the alpha-1,6 branch mannose residue. In some specific embodiments, enzymes or processes selectively affected (e.g., inhibited, modulated, or promoted) by a selective N-linked synthesis inhibitor include, by way of non-limiting example, enzymes or processes involved in the biosynthesis (e.g., N-linked glycan diversification) of complex N-linked glycans, such as alpha mannosidase III, which, in some instances, removes alpha-1,3 and alpha-1,6 mannose residues from alpha-1,6 branch mannose residues without prior GlcNAcTI addition of GlcNAc. In certain specific embodiments, enzymes or processes selectively affected (e.g., inhibited, modulated, or promoted) by a selective N-linked synthesis inhibitor include, by way of non-limiting example, GlcNAc transferases (e.g., I-V1, which, in certain instances, add GlcNAc residues to a trimannosyl core with up to 5 branches); GlcNAc transferase I or II (e.g., in the biosynthesis of complex N-linked glycans); extension of GlcNAc residues with additional monosaccharide linkages (e.g., other than those added by GLCNAcTIII); GlcNAC transferase I and/or IV (e.g., in a process for forming two branches on an alpha-1,3 mannose, such as in hybrid N-glycans); GlcNAC transferase III (e.g., as in a process of acting upon hybrid glycans to add a beta-1,4 GlcNAC to mannose attached beta-1,4 to the center beta-1,4 mannose in a trimannose core, which in certain instances leads to the inhibition of mannosidase 11 and GlcNACTIV, resulting in a hybrid glycan); GlcNAc transferase IV; GlcNAc transferase V; GlcNAc transferase III; GlcNAc transferase VI; and/or bisecting GlcNAc added by GlcNAcTIII. In some embodiments, provided herein is a selective N-linked glycan synthesis inhibitor that selectively modulates (e.g., promotes or inhibits) the formation of N-linked glycans selected from one or more of the N-linked glycans as follows: hybrid N-linked glycans; complex N-linked glycans; mono-antennary hybrid N-linked glycans, bi-antennary hybrid N-linked glycans; N-linked glycans bearing a high number of GlcNAcTV branches; N-linked glycans with core alpha-1,6 fucosylation to the GlcNAc attached to the Asn residue of a protein; and/or N-linked glycans with core alpha-1,3 fucosylation to the GlcNAc attached to the Asn residue of a protein. In specific embodiments, a selective inhibitor described herein optionally selectively inhibits any one or more of these early enzymes compared to other enzymes of the group, or any other enzyme involved in the biosynthesis of N-linked glycans.

In some embodiments, the late stage biosynthesis inhibitors inhibit one or more process in the late stage biosynthetic pathway, as described herein, but do not affect the biosynthesis of or N-linked glycan in the biosynthetic pathway prior to the late stage biosynthetic pathway. In various embodiments, an agent that does not affect the biosynthesis of or N-linked glycan(s) in biosynthetic pathway prior to the late stage biosynthetic pathway affects the non-late stage biosynthetic process or N-linked glycan(s) in a ratio of less than 1:2, less than 1:3, less than 1:4, less than 1:5, less than 1:8, less than 1:10, less than 1:15, less than 1:20, less than 1:25, less than 1:30, less than 1:40, less than 1:50, less than 1:100, when compared to the inhibition of a late stage biosynthetic process or N-linked glycan(s).

In some embodiments, a selective N-linked glycan inhibitor described herein selectively inhibits the enzyme β1,6N-acetylglucosaminyltransferase V (MGAT5), which is required for β1,6 NAc branched N-glycans attached to cell surface and secreted glycoproteins, and/or selectively inhibits for β1,6 NAc branched N-glycans attached to cell surface and secreted glycoproteins. In certain instances, amounts of MGAT5 glycan products are commonly increased in malignancies, and correlate with disease progression.

In certain embodiments, selective N-linked glycan inhibitors described herein modulate (e.g., promote or inhibit) the biosynthesis of N-linked glycans with an increased or decreased ability to bind with or otherwise associate with one or more proteins, one or more core proteins, one or more lectin, one or more growth factor, or the like. In specific embodiments, the selective N-linked glycan inhibitor described herein modulates (e.g., promotes or inhibits) the biosynthesis of an N-linked glycan with an increased or decreased ability to bind or associate with, e.g., transferrin, ribonuclease B, EGF Receptor, lamp1, N-cadherin, beta1-integrin, matriptase, an integrin, or the like. In some embodiments, selective N-linked glycan inhibitors described herein modulate (e.g., promote or inhibit) the biosynthesis of N-linked glycans associated to achieve one or more specific result. In specific embodiments, the selective N-linked glycan inhibitor described herein modulates (e.g., promotes or inhibits) the biosynthesis of a specific N-glycans to specifically and/or selectively vary, tune, or optimize the stability, solubility, cellular location, expression of, and/or activity of N-linked glycans and/or N-linked glycanated proteins produced. In various embodiments, a selective N-linked glycan biosynthesis inhibitor selectively modulates (e.g., promotes or inhibits) the biosynthesis of one N-linked glycan comprising antigen in a ratio of greater than 1000:1, greater than 500:1, greater than 250:1, greater than 100:1, greater than 50:1, greater than 25:1, greater than 20:1, greater than 10:1, greater than 5:1, greater than 3:1, or greater than 2:1 over one or more other O-linked glycan comprising antigen. (e.g., another enzyme involved in the N-linked biosynthetic pathway, and/or another enzyme involved in the biosynthetic pathway of a non-N-linked glycan).

In some embodiments, an N-linked glycan biosynthesis inhibitor described herein is a selective N-linked glycan biosynthesis that inhibits any specific transferase described herein over any one or more other transferase involved in the N-linked glycan biosynthetic pathway (e.g., over all other transferases involved in the N-linked glycan biosynthetic pathway), such as any transferase described or involved in the biosynthetic process in any of FIGS. 3-8. In certain embodiments, an N-linked glycan biosynthesis inhibitor described herein is a selective N-linked glycan biosynthesis inhibitor that inhibits any specific transferase described herein as being involved in the N-linked glycan biosynthetic pathway over any one or more transferase involved in the biosynthetic pathway of a non-N-linked glycan (e.g., O-linked glycan, glycosaminoglycan, ganglioside, or the like). In some embodiments, biosynthetic modulators (e.g., inhibitors) described herein include agents that directly or indirectly inhibit the biosynthesis of the glycan. In certain instances, the modulator (e.g., inhibitor) directly modulates (e.g., inhibits) formation of a glycan structure (e.g., one as described herein) or an enzyme involved in the biosynthetic pathway. In some instances, the modulator (e.g. inhibitor) indirectly modulates (e.g., by acting on an upstream glycan structure or enzyme) formation of a glycan structure (e.g., one as described herein) or an enzyme involved in the biosynthetic pathway.

In certain embodiments, a selective N-linked glycan biosynthesis inhibitor is selective for (i.e., directly or indirectly inhibits the activity of) a specific enzyme (e.g., transferase) in a ratio of greater than 1000:1 over one or more other enzyme (e.g., another enzyme involved in the N-linked biosynthetic pathway, and/or another enzyme involved in the biosynthetic pathway of a non-O-linked glycan). In specific embodiments, a selective N-linked glycan biosynthesis inhibitor is selective for (i.e., directly or indirectly inhibits the activity of) a specific enzyme in a ratio of greater than 500:1 over one or more other enzyme (e.g., another enzyme involved in the N-linked biosynthetic pathway, and/or another enzyme involved in the biosynthetic pathway of a non-N-linked glycan). In specific embodiments, a selective N-linked glycan biosynthesis inhibitor is selective for (i.e., directly or indirectly inhibits the activity of) a specific enzyme in a ratio of greater than 250:1, greater than 100:1, greater than 50:1, greater than 25:1, greater than 20:1, greater than 10:1, greater than 5:1, greater than 3:1, or greater than 2:1 over one or more other enzyme (e.g., another enzyme involved in the N-linked biosynthetic pathway, and/or another enzyme involved in the biosynthetic pathway of a non-N-linked glycan).

Moreover, in certain embodiments, provided herein is an N-linked glycoprotein or N-linked glycan that was prepared by modifying the biosynthesis thereof with any selective inhibitor described herein.

In some embodiments, a selective N-linked glycan biosynthesis inhibitor described herein is a selective inhibitor of the initiation of precursor synthesis. In certain embodiments, a selective N-linked glycan biosynthesis inhibitor is a selective polymerization (e.g. polylactosamine polymerization, mannosylation) inhibitor. In certain embodiments, a selective N-linked glycan biosynthesis inhibitor is a selective glycophosphorylation inhibitor. In certain embodiments, a selective N-linked glycan biosynthesis inhibitor is a selective sialyl transferase inhibitor. In certain embodiments, a selective N-linked glycan biosynthesis inhibitor is a selective inhibitor of GlcNAc-TV. In certain embodiments, a selective N-linked glycan biosynthesis inhibitor is a selective oligosaccharyl transferase inhibitor. In certain embodiments, a selective N-linked glycan biosynthesis inhibitor is a selective iGnT inhibitor. In certain embodiments, a selective N-linked glycan biosynthesis inhibitor is a selective inhibitor of a transporter or chaperone that mediates N-linked glycan synthesis.

In certain instances, the biosynthesis on N-glycans comprises four distinct phases. Phase 0 provides the synthesis of building blocks, for example, sugar donors, dolichol, etc. Phase I provides the synthesis of dolichol-P-P-GlcNAc₂Man₉Glc₃. Phase II provides the processing and trimming of an N-linked glycan. Phase III provides the branching and modification of complex N-glycans. In certain embodiments described herein is an N-linked glycan biosynthesis inhibitor that is selective for Phase 0 inhibition. In some embodiments, an N-linked glycan biosynthesis inhibitor described herein is selective for Phase I inhibition. In certain embodiments, an N-linked glycan biosynthesis inhibitor described herein is selective for Phase II inhibition. In some embodiments, an N-linked glycan biosynthesis inhibitor described herein is selective for Phase III inhibition. In some embodiments, an N-linked glycan biosynthesis inhibitor selectively inhibits one or more process as described in any of FIGS. 3-8.

### Cellular Activity

In some embodiments, provided herein is a N-linked glycan biosynthesis modulator (e.g., a selective biosynthesis inhibitor) having suitable cell availability and/or bioavailability to significantly effect the in cyto and/or in vivo biosynthesis of a N-linked glycan (e.g., a specific glycolipid in certain instances wherein a selective glycolipid synthesis modulator is utilized) when the N-linked glycan biosynthesis modulator is administered to a cell or individual, respectively. In certain instances, a significant effect is one wherein a measurable effect, a statistically significant effect, and/or a therapeutic effect is provided to the cell or individual. In certain specific embodiments, the specific glycolipid modulator is substantially cell permeable (e.g., when in contact with a cell, a significant percentage/amount of the modulator permeates the cell membrane). In some embodiments, the N-linked glycan synthesis modulator (e.g., promoter or inhibitor) has cellular activity (e.g., when put in contact with a cell, the modulator significantly (e.g., therapeutically significantly, physiologically significantly, statistically significantly, or the like) affects cellular N-linked glycan synthesis according to any manner described herein. In some embodiments, the N-linked glycan biosynthesis modulator provides a statistically significant effect and/or therapeutic effect in a cell or individual at a non-toxic concentration, a substantially non-toxic concentration, a concentration below LC₅₀, a concentration below LC₂₀, a concentration below LC₀₁, or the like.

In certain instances, N-linked biosynthesis modification (Inhibition/promotion) is accomplished most effectively through a small molecule that can penetrate a cell in order to reach its target. In some embodiments, N-linked glycan biosynthesis inhibitors described herein with cellular activity are capable of altering the function of a biosynthetic enzyme or a regulator of one in an intact cell in culture or in an intact organism.

### Compounds

In certain embodiments, the N-linked glycan synthesis inhibitors described herein modulate (e.g., promote or inhibit) one or more of the synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), a transporter (e.g., a transporter for (Man α/β)₆-(GlcNAcβ)₂-Asn), one or more of the Man being optionally phosphorylated.

In certain embodiments, N-linked glycan biosynthesis inhibitors described herein are small molecule organic compounds. Thus, in certain instances, N-linked glycan biosynthesis inhibitors utilized herein are not polypeptides or carbohydrates. In some embodiments, in certain embodiments, a small molecule organic compounds has a molecular weight of less than about 2,000 g/mol, less than about 1,500 g/mol, less than about 1,000 g/mol, less than about 700 g/mol, or less than about 500 g/mol. In some embodiments, the N-linked glycan biosynthesis inhibitors are non-carbohydrate small molecules. In some embodiments, the N-linked glycan biosynthesis inhibitors are non-carbohydrate organic compounds. In some embodiments, the N-linked glycan biosynthesis inhibitors are non-carbohydrate small molecule organic compounds.

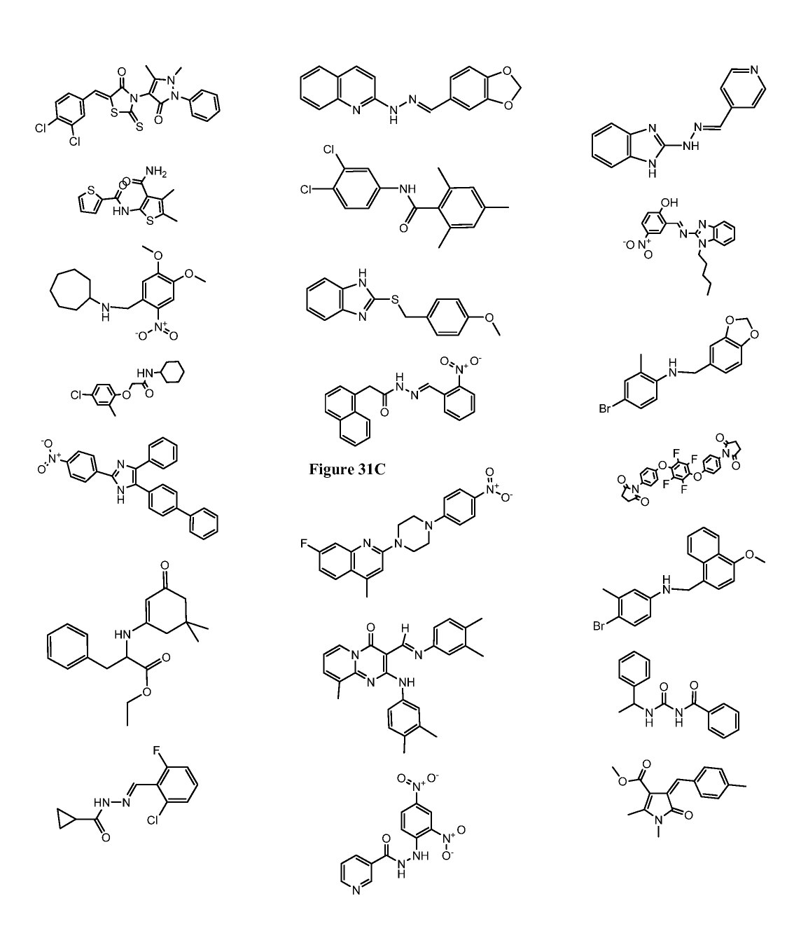

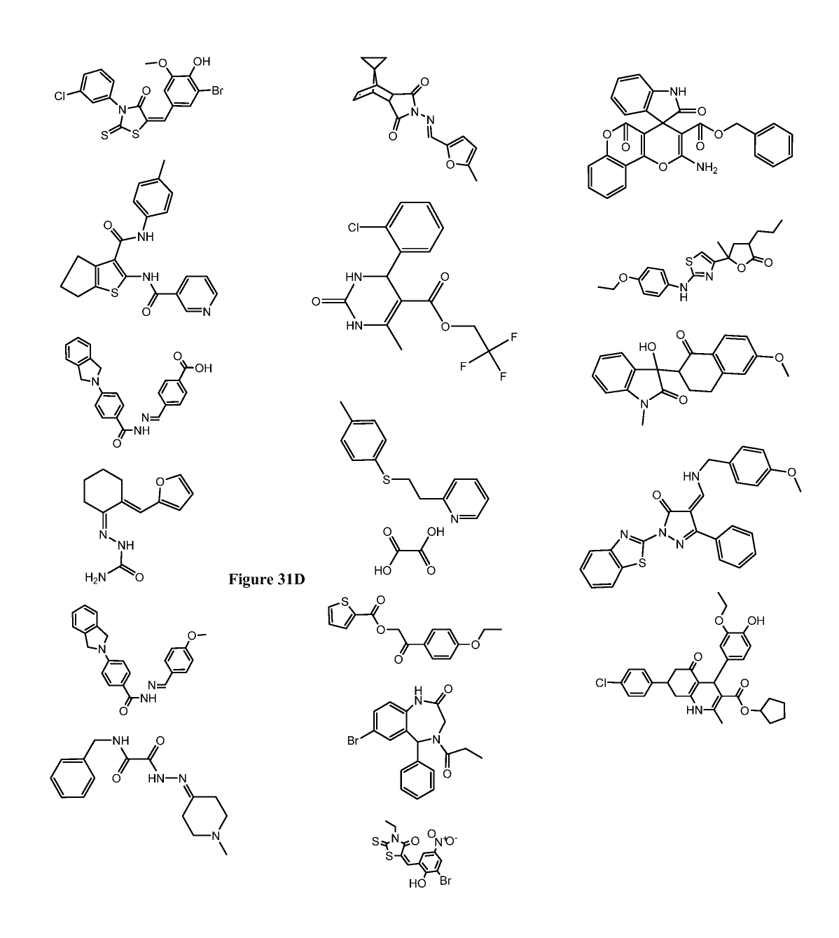

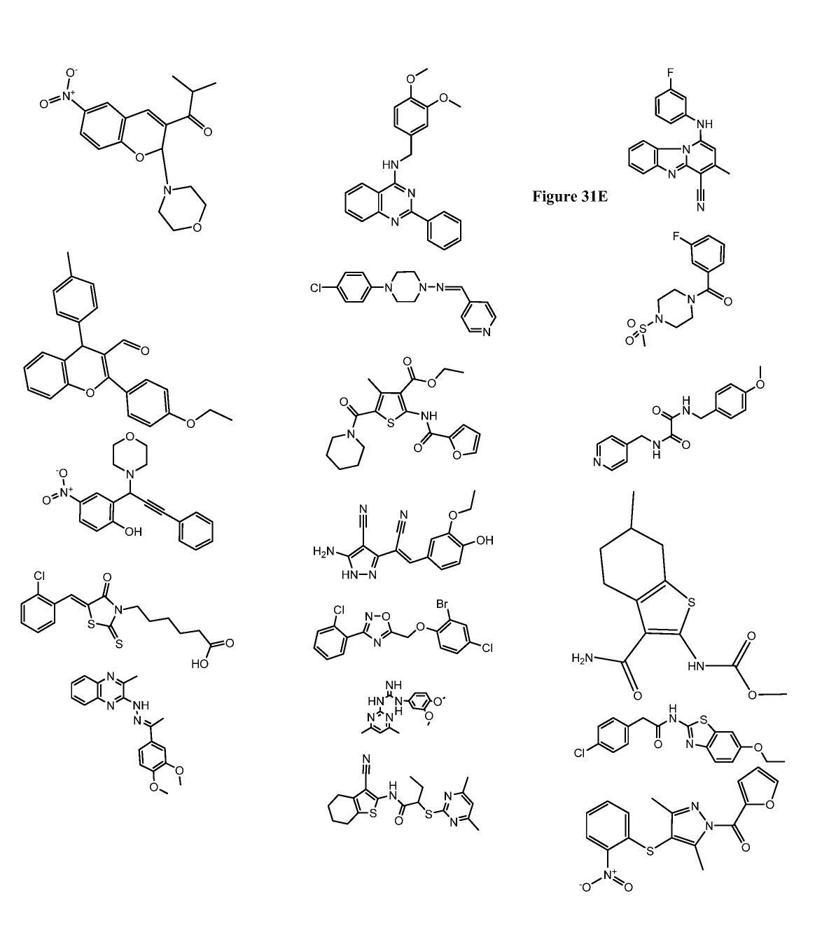

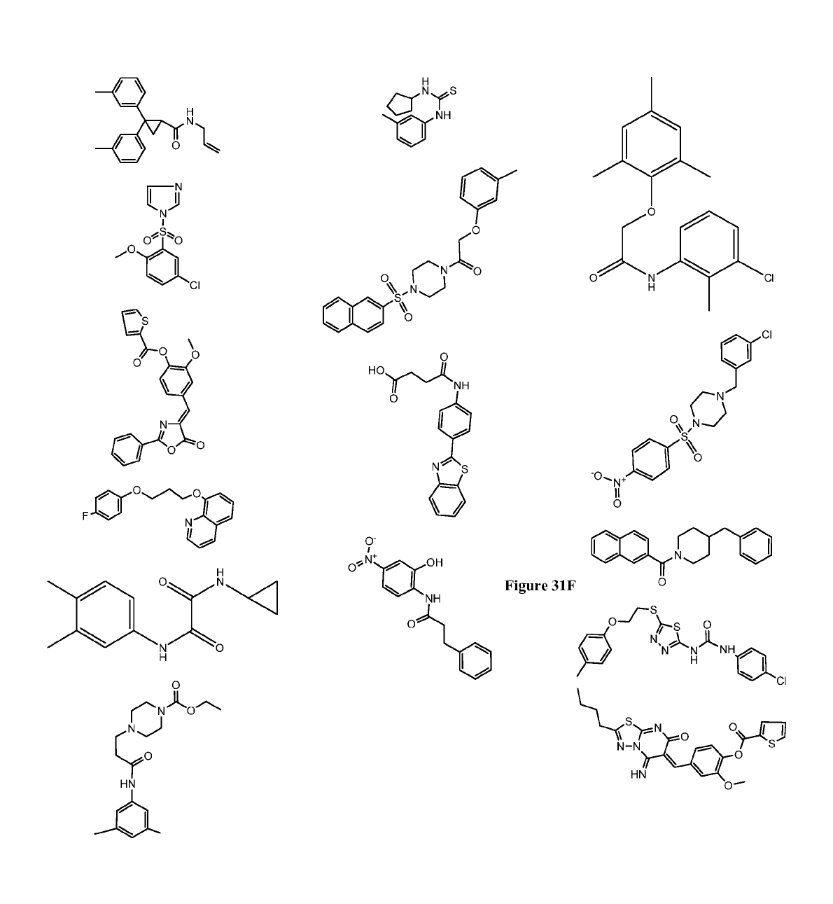

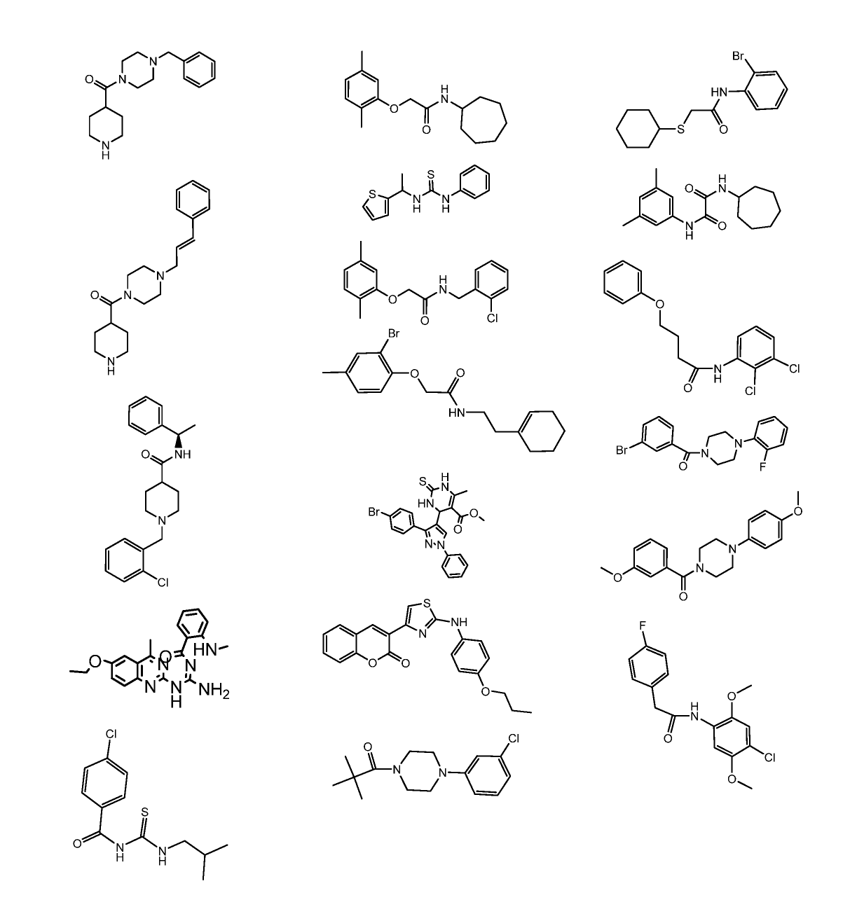

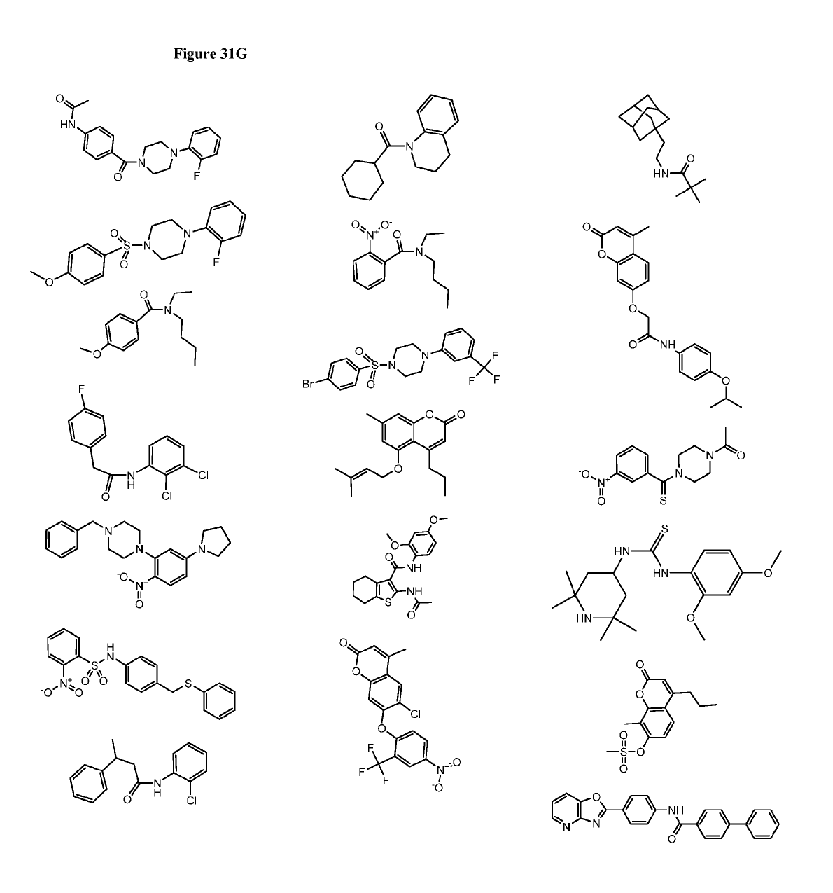

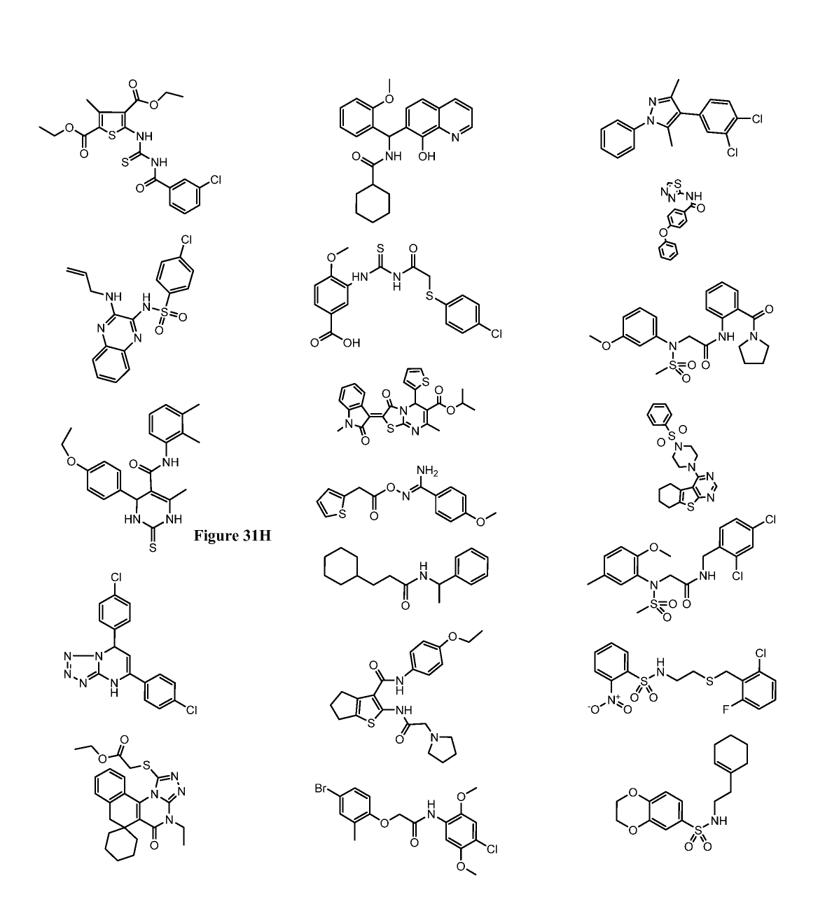

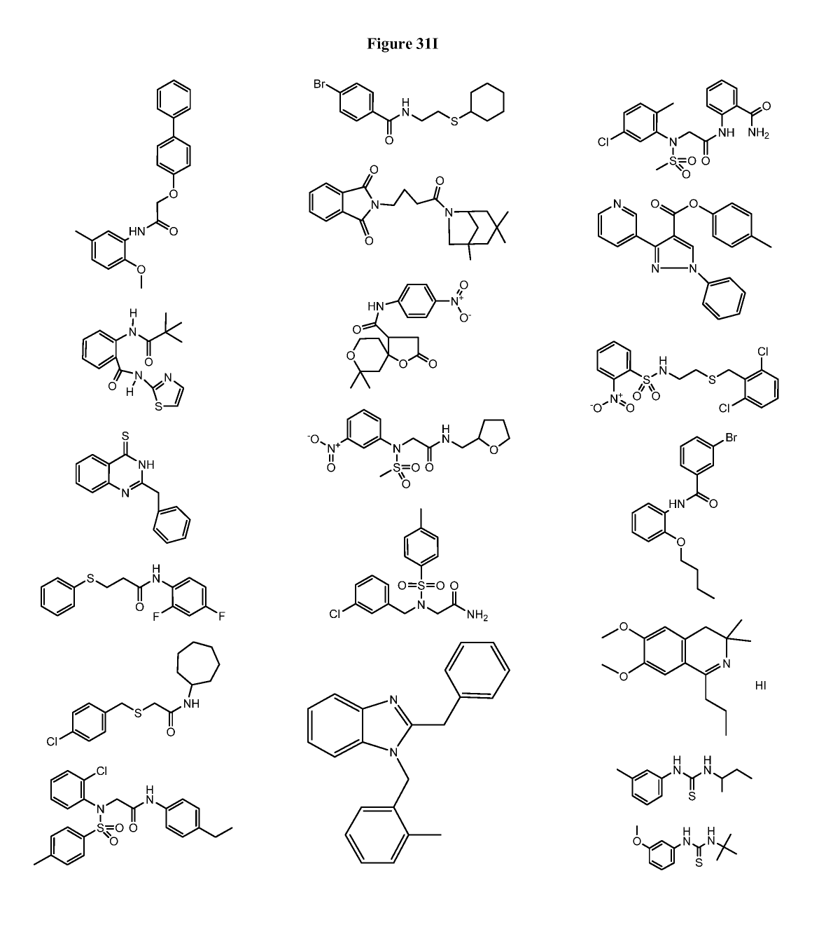

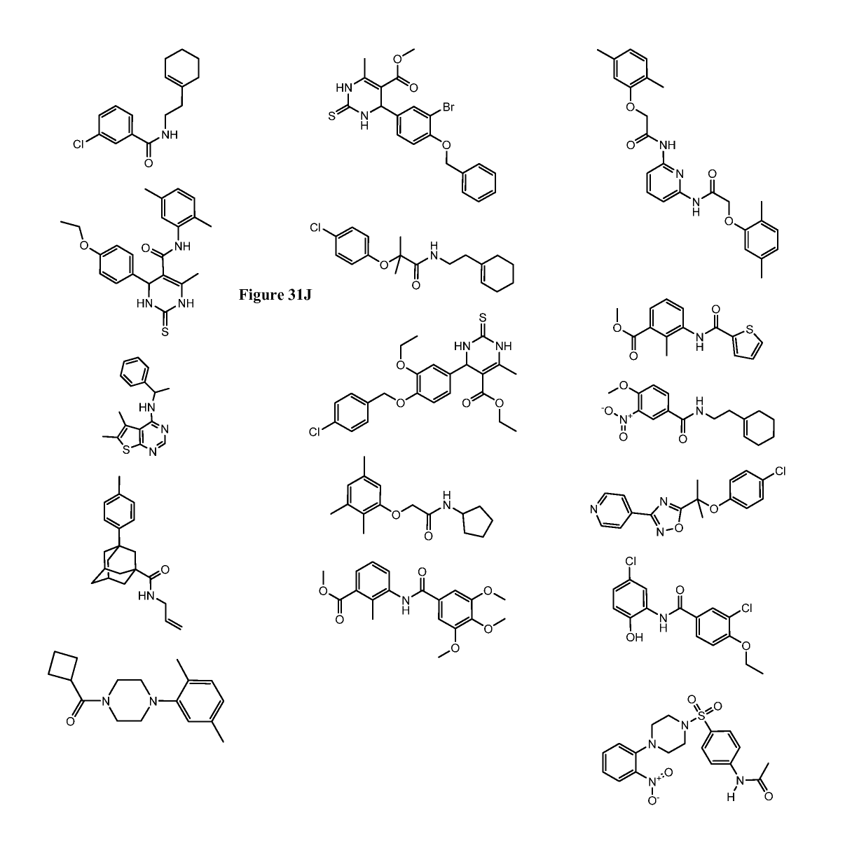

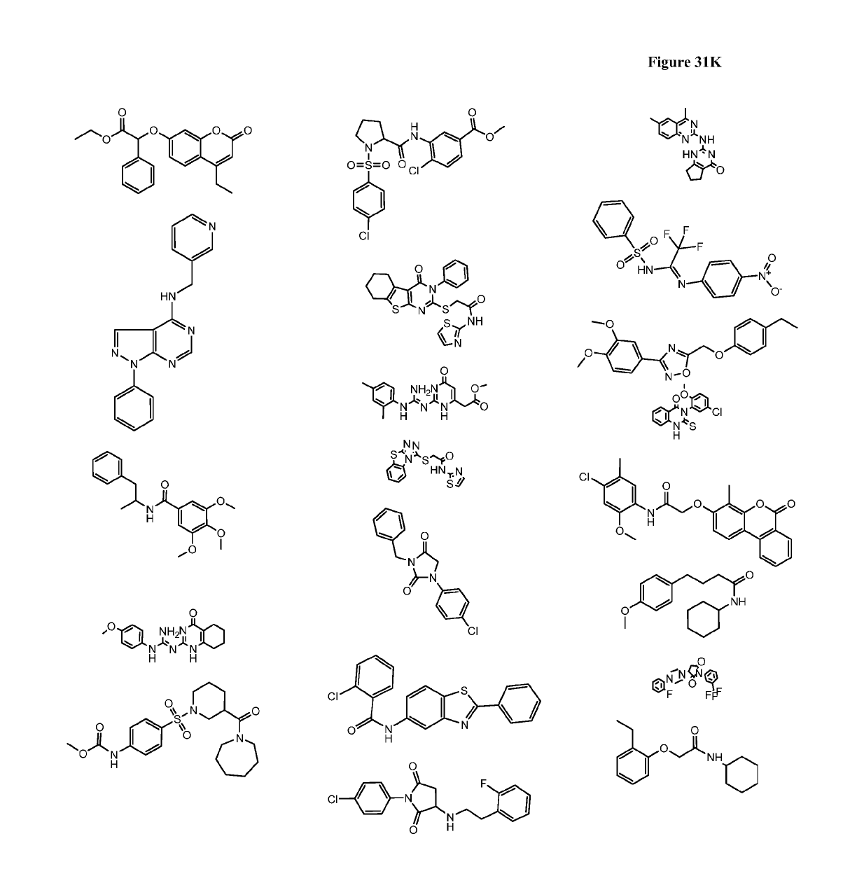

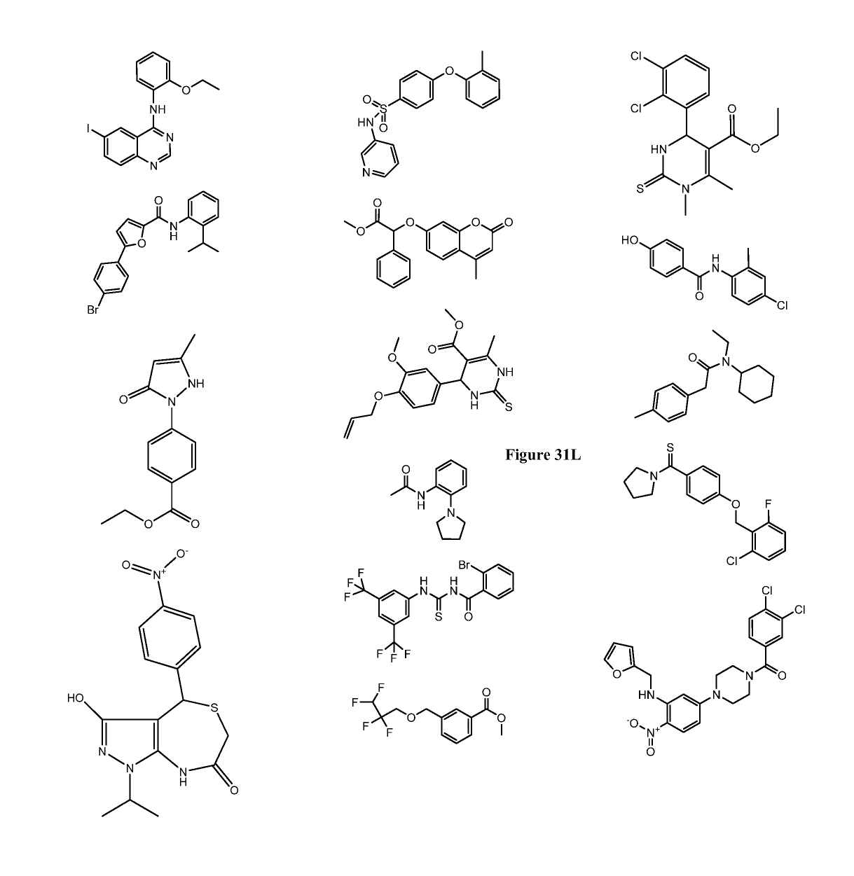

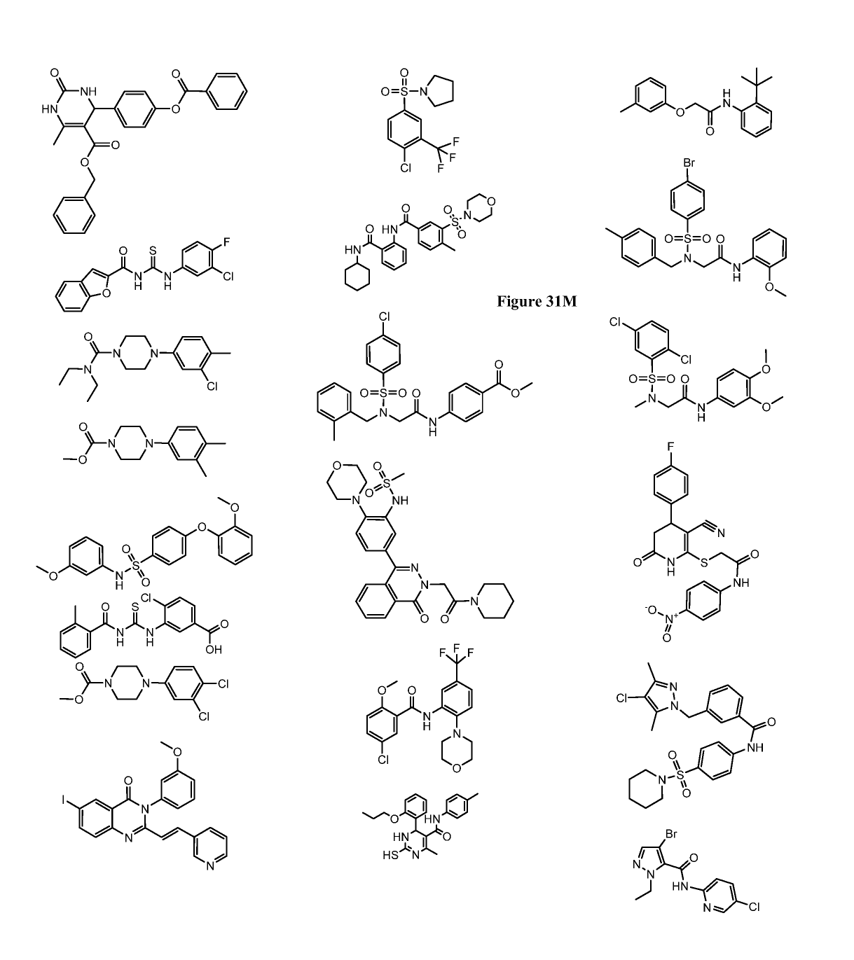

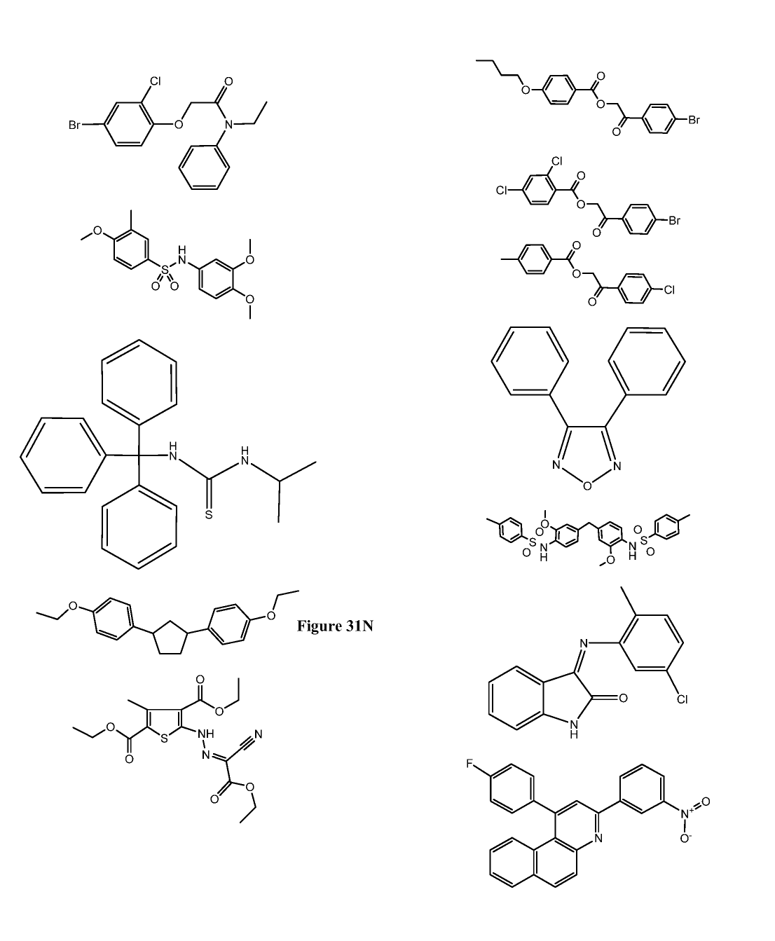

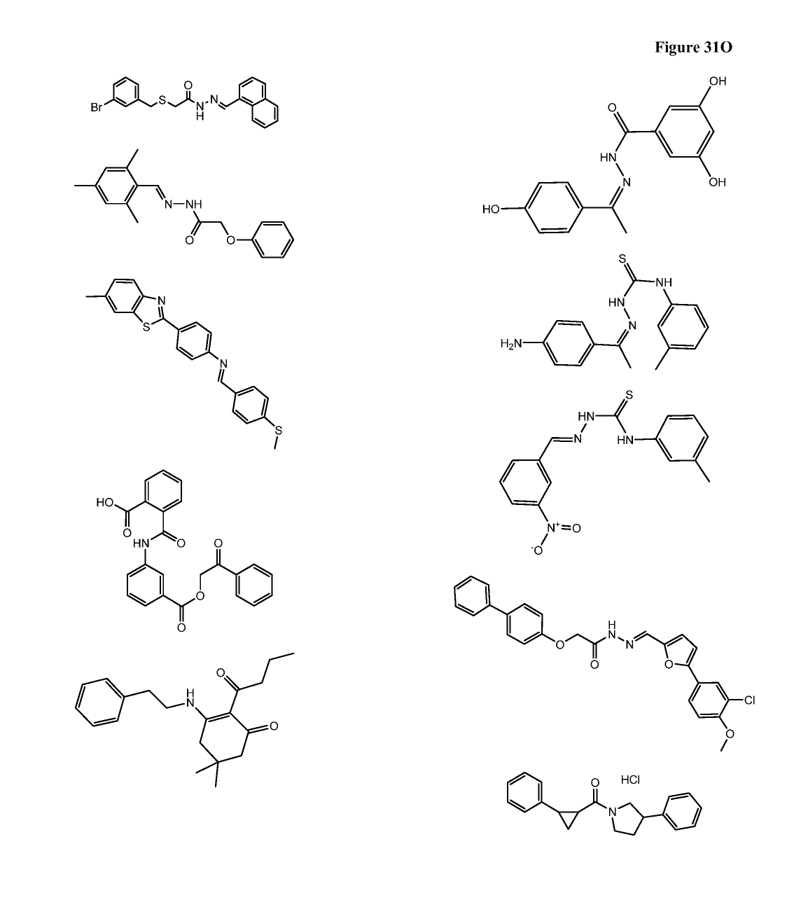

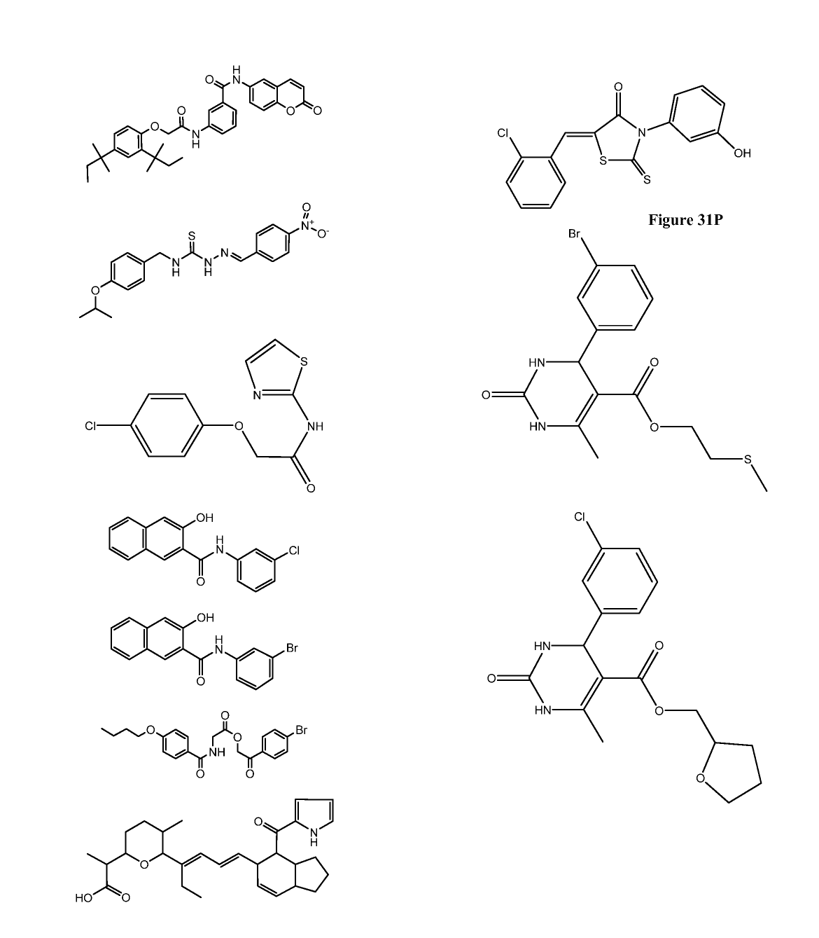

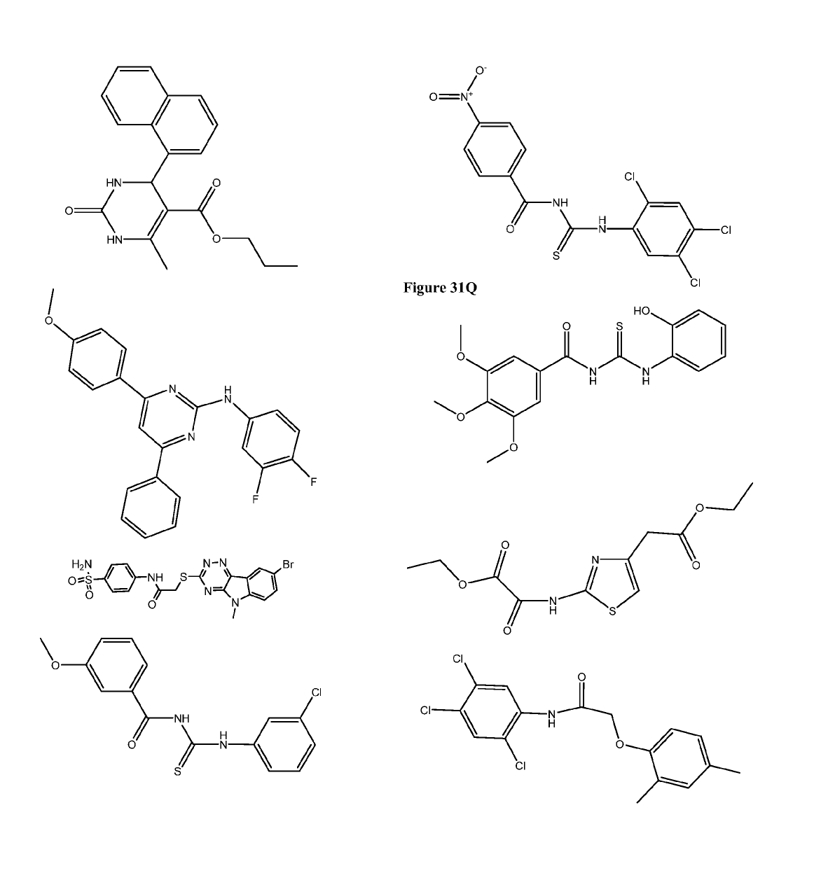

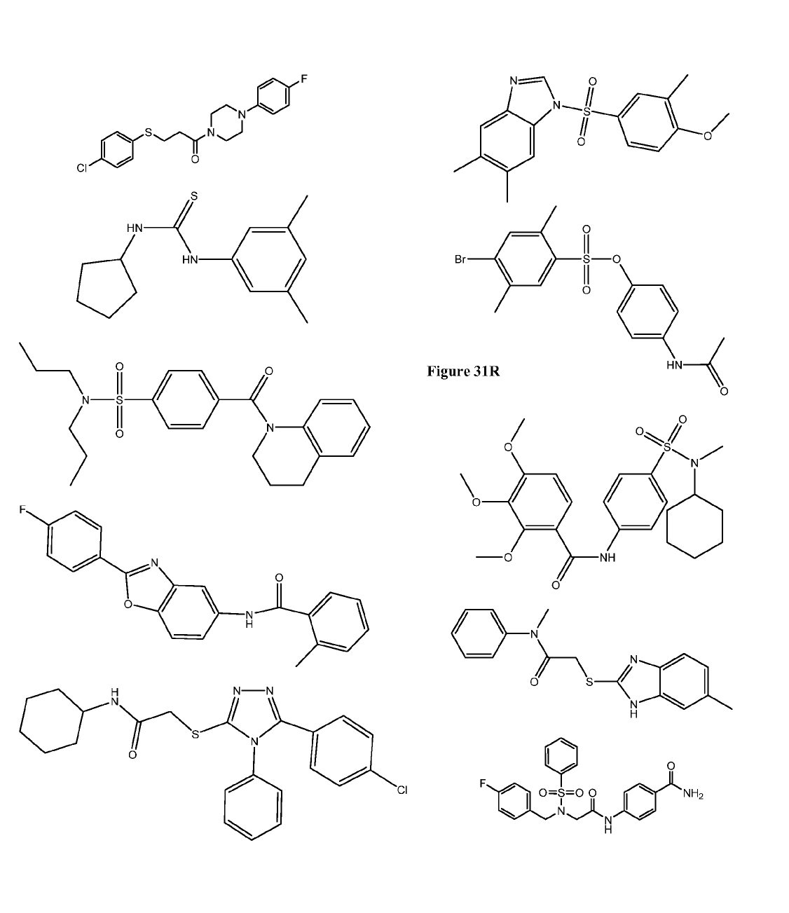

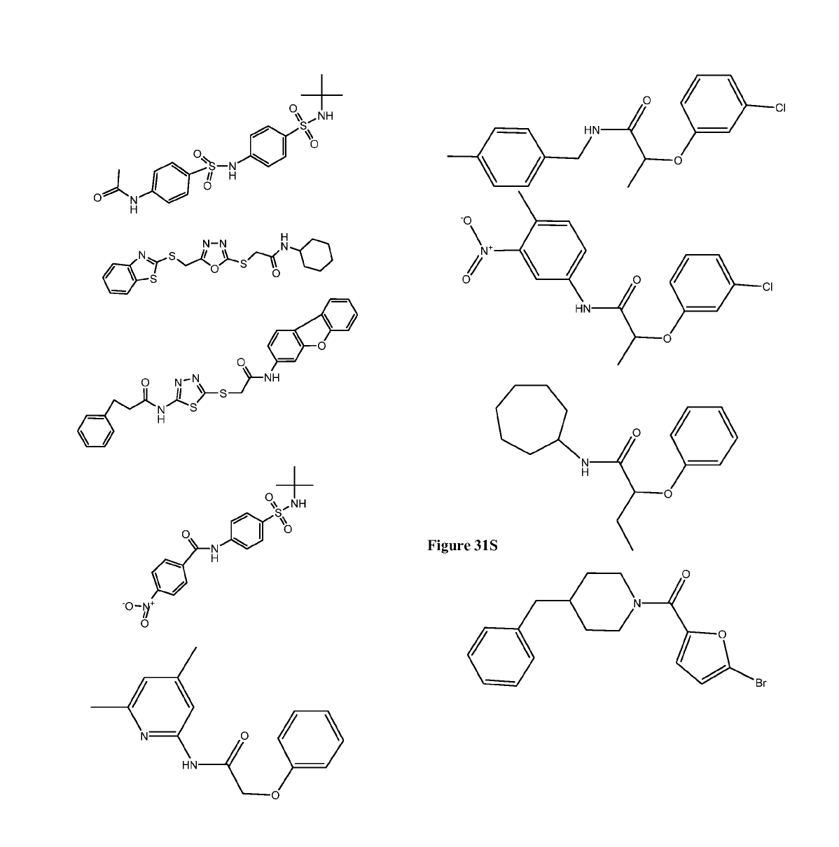

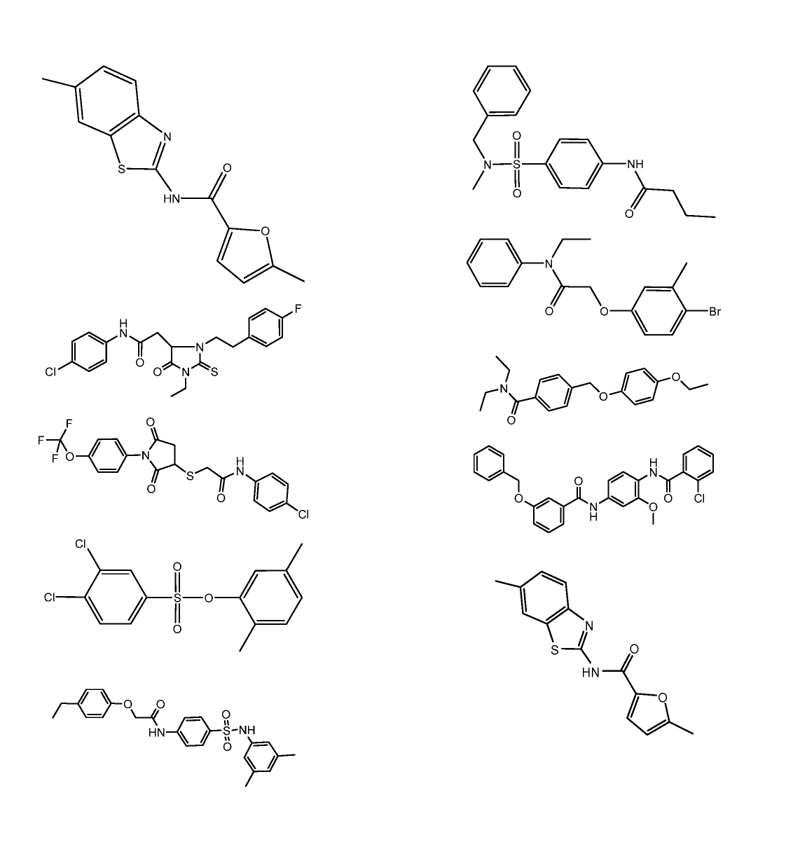

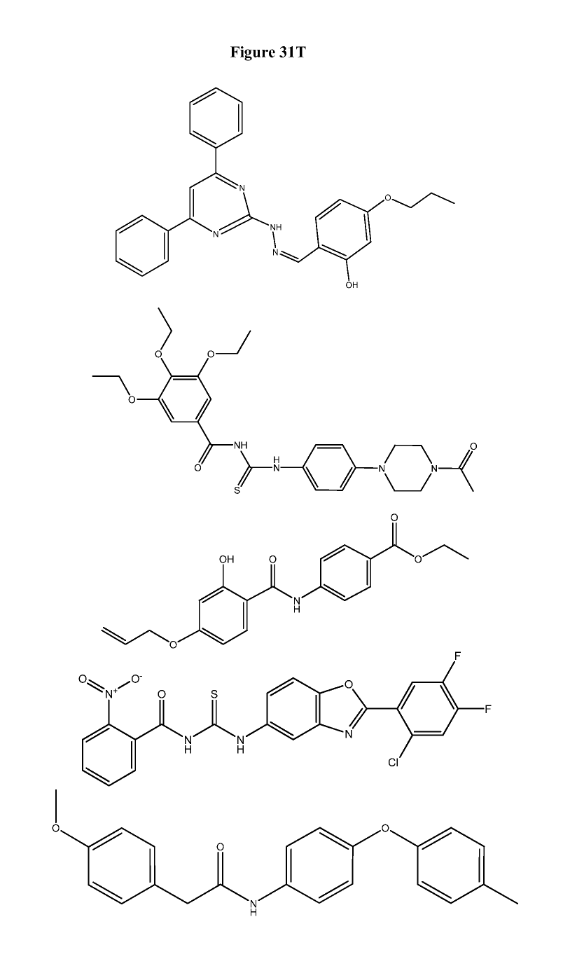

In some embodiments, selective inhibitors of N-linked glycan biosynthesis includes any compound of FIGS. 31A-31T. Incubating compounds of FIGS. 31A-31T in cells were observed to inhibit glycan-PHA binding, but did not demonstrate a significant inhibition of glycan-FGF binding (non-inhibitory against GAG, HS biosynthesis), glycan-WGA binding (non-inhibitory against Sialic acid containing and terminal GlcNAc glycans), or glycan-CTB binding (non-inhibitory against ganglioside biosynthesis). In certain embodiments, selective inhibitors of N-linked glycan biosynthesis include, but are not limited to, the following compounds: N-(2,3-dimethylphenyl)-4-(4-ethoxyphenyl)-6-methyl-2-thioxo-1,2,3,4-tetrahydropyrimidine-5-carboxamide (1); 3-(2-(2,4-di-tert-pentylphenoxy)acetamido)-N-(2-oxo-2H-chromen-6-yl)benzamide (2); (E)-N-(4-isopropoxybenzyl)-2-(4-nitrobenzylidene)hydrazinecarbothioamide (3); N-(4-chlorophenyl)-2-(1-ethyl-3-(4-fluorophenethyl)-5-oxo-2-thioxoimidazolidin-4-yl)acetamide (4); cyclopentyl 7-(4-chlorophenyl)-4-(3-ethoxy-4-hydroxyphenyl)-2-methyl-5-oxo-1,4,5,6,7,8-hexahydroquinoline-3-carboxylate (5); (Z)-2-((2-(4,6-diphenylpyrimidin-2-yl)hydrazono)methyl)-5-propoxyphenol (6); 7-bromo-5-phenyl-4-propionyl-4,5-dihydro-1H-benzo[e][1,4]diazepin-2(3H)-one (7); N-(3-chlorophenyl)-3-hydroxy-2-naphthamide (8); N-(3-bromophenyl)-3-hydroxy-2-naphthamide (9); N-(3-(p-tolylcarbamoyl)-5,6-dihydro-4H-cyclopenta[b]thiophen-2-yl)nicotinamide (10); (Z)-1-(benzo[d]thiazol-2-yl)-4-[((4-methoxybenzylamino)methylene)-3-phenyl-1H-pyrazol-5(4H)-one (11); (Z)-5-amino-3-(1-cyano-2-(3-ethoxy-4-hydroxyphenyl)vinyl)-1H-pyrazole-4-carbonitrile (12); 5-methyl-N-(6-methylbenzo[d]thiazol-2-yl)furan-2-carboxamide (13). In some embodiments, other inhibitors of N-linked glycan biosynthesis, including selective biosynthesis inhibitors, include other compounds identified according to any process described herein.

In certain embodiments, N-linked glycan biosynthesis inhibitors described herein are non-carbohydrate small molecule compounds. Carbohydrates tend to be hydrophilic due to the polyhydroxyls and therefore do not diffuse into cells efficiently. In some instances, carbohydrates have pharmacokinetic and pharmacodynamic properties in animals that are inappropriate for therapeutic drug effects. Further, the hydroxyls are reactive and may make carbohydrates difficult and expensive to synthesize. In certain instances, carbohydrates are not known to cross the blood-brain barrier. In certain instances, noncarbohydrate small molecules are much less likely to be immunogenic or immunoreactive than are carbohydrates.

Carbohydrates include polhydroxyaldehydes, polyhydroxyketones and their simple derivatives or larger compounds that can be hydrolyzed into such units. Carbohydrates also include polhydroxyaldehydes, polyhydroxyketones and their simple derivatives that have been modified such that when they enter cells they are reconverted into polhydroxyaldehydes, polyhydroxyketones. Carbohydrates also include sugar mimetics such as imino structures and alkaloids that inhibit glycosidases such as Deoxynojirimycin, Castanospermine, Australine, Deoxymannojirimycin, Kifunensen, Swainsonine and Mannostatin (page 709 of Essentials of Glycobiology second edition 2008 CSHL Press, CSH, New York.) Non carbohydrate small molecules include, e.g., organic compounds containing less than 3 linked hydroxyl groups with a molecular weight of less than 2000 Daltons.

Modulators (e.g., inhibitors) of glycan synthesis include agents that act directly on the relevant biosynthetic enzymes or indirectly on other targets (e.g. protein kinase, phosphatase, transporter, GPCR, ion channel, hormone receptor, protease, etc.) that would alter the structure of the glycans though effects on biosynthetic (anabolic) enzymes or degradative (catabolic) enzymes.

### GENERAL DEFINITIONS

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding is accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In some instances, the result is the alteration of or the disruption of the structure of endogenous N-linked glycan such that the binding ability, signaling ability or combination thereof of the N-linked glycan is inhibited or reduced. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Certain administration techniques employed with the agents and methods described herein are discussed in, e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, an N-linked glycan modulator agent described herein or a pharmaceutically active metabolite or residue thereof. Particularly favored prodrugs are those that increase the bioavailability of the N-linked glycan modulator agents described herein when such agents are administered to a patient (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The symbolic nomenclature used herein follows the “Symbol and Text Nomenclature for Representation of Glycan Structure” as promulgated by the Nomenclature Committee for the Consortium for Functional Glycomics, as amended on October 2007.

### Methods

Provided in certain embodiments herein is a process for modifying the structure of an N-linked glycan on a core protein comprising contacting a cell that translationally produces at least one core protein having at least one attached N-linked glycan with an effective amount of any N-linked glycan synthesis inhibitor described herein. In some embodiments, the N-linked glycan synthesis inhibitor is a selective N-linked glycan synthesis inhibitor, as described herein. In some embodiments, the selective N-linked glycan synthesis inhibitor is a modulator of one or more of (e.g., promotes one or more of, or inhibits one or more of) the synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α1,2-glucosidase I, α1,3-glucosidase II, α-1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), sulfation (e.g., N or O sulfation), acetylation (e.g., N or O acetylation), phosphorylation, or a transporter (e.g., a transporter for (Man α/β)₆-(GlcNAcβ)₂-Asn), one or more of the mannose residues (Man) being optionally phosphorylated.

In some embodiments, an N-linked glycan synthesis inhibitor modulates (e.g., promotes or inhibits) a glycosyltransferase (e.g., a UDP-GlcNAcT, GDP mannosyl transferase). In some embodiments, an inhibitor of a glycosyltransferase inhibits the synthesis of the precursor unit and/or the initiation of precursor unit synthesis. In some embodiments an N-linked glycan synthesis inhibitor modulates (e.g., promotes or inhibits) an oligosaccharyl transferase (e.g., D-OST). In some embodiments, an inhibitor of an oligosaccharyl transferase inhibits the attachment of a precursor unit to an Asn residue on a core protein. In some embodiments, an N-linked glycan synthesis inhibitor modulates (e.g., promotes or inhibits) a glycosidase (e.g., α1,2-glucosidase I, α1,3-glucosidase II, α-1,2-mannosidase, α1,2-specific Golgi mannosidase I, Golgi α1,6-mannosidase II, N-acetylglucosamine-1-phosphodiester alpha-N-acetylglucosaminidase). In some embodiments, an inhibitor of a glycosidase inhibits further processing (e.g., cleavage of residues) of a precursor unit attached to a core protein. In some embodiments, an N-linked glycan synthesis inhibitor modulates (e.g., promotes or inhibits) polymerization of a pentasaccharide core (e.g., promotes or inhibits GlcNAc-TI, GlcNAc-TII, GlcNAc-TIV, GlcNAc-TV, i-GnT, β-1,3-N-acetylglucosaminyltransferase, β-1,4-galactosyltransferase). In some embodiments, an N-linked glycan synthesis inhibitor modulates (e.g., promotes or inhibits) further modification of an N-linked glycan. In some embodiments, an inhibitor of further modification of a glycan inhibits, e.g., an i-extension enzyme, (e.g., iGnT), a polylactosamine extension enzyme, (e.g., β1-4-galactosyl transferase IV(β4Gal-TIV)) a fucosyl transferase (e.g., FucTVII, FucTIV), or a sialyl transferase (e.g., ST3GalIV, ST3GalVI), or a combination thereof.

In some instances, an N-linked glycan synthesis inhibitor alters or disrupts (e.g., synthesis of the β-1,6 branched N-linked glycans, e.g., synthesis of N-acetylglucosamine linked β-1,6- to an α1,3-mannose) the nature of the N-linked glycan such that it inhibits the binding, signaling, or a combination thereof of any protein subject to N-linked glycan binding, signaling or a combination thereof, compared to binding in the absence of an N-linked glycan synthesis inhibitor. In some instances, alteration or disruption of the nature of the N-linked glycan alters or modulates the presence of complex β-1,6-branched N-linked glycans in any protein subject to N-linked glycan binding, signaling or a combination thereof, compared to binding in the absence of an N-linked glycan synthesis inhibitor. In some instances, the alteration or modulation of the presence of complex β-1,6-branched N-linked glycans inhibits the binding, signaling, or a combination thereof of any protein subject to N-linked glycan binding, signaling or a combination thereof, compared to binding in the absence of an N-linked glycan synthesis inhibitor.

In some instances, an N-linked glycan synthesis inhibitor alters or disrupts the nature of an N-linked glycan such that it inhibits the binding, signaling, or a combination thereof of any lectin (including polypeptides) subject to β-1,6-branched N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments the CAM is an exogenous CAM, e.g., bacterial lectins. In certain embodiments, the CAM is an endogenous CAM and includes, by way of non-limiting examples, E-selectin, L-selectin or P-selectin.

In some instances, an N-linked glycan synthesis inhibitor alters or disrupts the nature of an N-linked glycan such that it inhibits the binding, signaling, or a combination thereof of any lectin e.g., galectin-3 or any one or more galectin, (including polypeptides) subject to binding, signaling or a combination thereof to a β-1,6-branched N-linked glycan modified with N-acetyllactosamine, compared to binding in the absence of an N-linked glycan synthesis inhibitor. In some instances, an N-linked glycan synthesis inhibitor alters or disrupts the nature of an N-linked glycan such that it inhibits the binding, signaling, or a combination thereof of a protein, (e.g., integrin, matriptase and/or N-cadherin) subject to β-1,6-branched N-linked glycan binding, signaling or a combination thereof, compared to binding in the absence of an N-linked glycan synthesis inhibitor.

In certain embodiments, the cell is present in an individual (e.g., a human) diagnosed with a disorder mediated by N-linked glycan biosynthesis. In certain instances, the disorder mediated by N-linked glycan biosynthesis is a cancer, a tumor, undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, organomegaly (e.g., hepatosplenomegaly), amyloidosis, skeletal abnormalities, odontoid hypoplasia, hydrops fetalis, inflammation, sialuria, sialidosis, thrombocytopenia, leukopenia, tumorous calcinosis, Ehlers-Danlos syndrome, Walker Warburg syndrome, a wound, or the like. In some embodiments, the cell is present in a human diagnosed with cancer. In certain embodiments, the cell is present in an individual (e.g., a human) diagnosed with abnormal angiogenesis and/or undesired angiogenesis. In some embodiments, the cell is present in an individual (e.g., a human) diagnosed with a lysosomal storage disease (e.g., mucopolysaccharidosis (MPS)). In some embodiments, the individual is diagnosed with MPS I, MPS II, or MPS III. In some embodiments, the cell is present in an individual (e.g., a human) diagnosed with amyloidosis, a spinal cord injury, hypertriglyceridemia, inflammation, or the like. In some embodiments, the disorder mediated by N-linked glycan biosynthesis is an inflammatory disease (e.g., an acute or chronic inflammatory disorder) including but not limited to Crohn's disease, reactive arthritis, including Lyme disease, insulin-dependent diabetes, organ-specific auto immunity, Hashimoto's thyroiditis and Grave's disease, contact dermatitis, psoriasis, organ transplant rejection, graft rejection, graft versus host disease, sarcoidosis, atopic conditions, gastrointestinal allergies, including food allergies, pancreatitis, eosinophilia, conjunctivitis, glomerular nephritis, multiple vasculitides, myasthenia gravis, asthma, chronic obstructive pulmonary disease, myocardial infarction, stroke, transplant rejection, reperfusion injury, autoimmune disease (e.g, Ankylosing spondylitis, systemic lupus erythematosus (SLE), or the like) inflammatory bowel disease, psoriasis, arthritis (including, e.g., rheumatoid arthritis), allergic rhinitis, berillium disease, bronchiectasis, bronchitis, bronchopneumonia, cystic fibrosis, diphtheria, dyspnea, emphysema, allergic bronchopulmonary aspergillosis, pneumonia, acute pulmonary edema, pertussis, pharyngitis, atelectasis, Wegener's granulomatosis, Legionnaires disease, pleurisy, rheumatic fever, sinusitis or the like. In certain embodiments, the disorder mediated by N-linked glycan biosynthesis is a lysosomal storage disease (LSD) such as sialidosis (Type I, Type II) and fucosidosis. In some embodiments, the disorder mediated by N-linked glycan biosynthesis is an infectious disease targeting N-linked glycans of either host or the pathogen. In certain instances, this could affect the infection process or the pathogen life cycle. In various embodiments, pathogens may include bacteria, viruses (e.g., HIV, influenza, or the like) and fungi.

In some embodiments, the cell is present in an individual (e.g., human) diagnosed with a carcinoma or adenocarcinoma. In some embodiments, the cell is present in an individual diagnosed with pancreatic cancer, myeloma, ovarian cancer, hepatocellular cancer, breast cancer, colon carcinoma, or melanoma. In certain embodiments, the cell is a pancreatic cancer cell, myeloma cell, ovarian cancer cell, hepatocellular cancer cell, breast cancer cell, colon carcinoma cell, renal cell carcinoma, carcinoma of the gut, lung or urogenital tract, or melanoma cell. In some embodiments, the cell is present in an individual (e.g., human) diagnosed with an inflammatory disease, LSD, infectious disease, or the like.

In some embodiments, the cell is present in an individual (e.g., human) diagnosed with an infectious or viral disease including, by way of non-limiting example, herpes, diphtheria, papilloma virus, hepatitis, HIV, coronavirus, or adenovirus.

In certain embodiments, N-linked glycan synthesis inhibitors described herein are small molecule organic compounds. In certain instances, N-linked glycan synthesis inhibitors utilized herein are not polypeptides or carbohydrates. In some embodiments, a small molecule organic compound has a molecular weight of less than about 2,000 g/mol, less than about 1,500 g/mol, less than about 1,000 g/mol, less than about 700 g/mol, or less than about 500 g/mol. In some embodiments, the N-linked glycan synthesis inhibitors are non-carbohydrate small molecules. In some embodiments, the N-linked glycan synthesis inhibitors are non-carbohydrate organic compounds. In some embodiments, the N-linked glycan synthesis inhibitors are non-carbohydrate small molecule organic compounds.

In certain embodiments, provided herein is a method of treating a disorder mediated by N-linked glycans by administering to an individual (e.g., a human) in need thereof a therapeutically effective amount of any N-linked glycan synthesis inhibitor described herein. In some embodiments, the N-linked glycan synthesis inhibitor is a modulator (e.g., inhibitor or promoter) of a glycosyl transferase, an oligosaccharyl transferase, a mannosidase, a glucosidase, a phosphotransferase, a sialyl transferase, a fucosyl transferase, an acetyglucosaminyl transferase, an i-extension enzyme, a α sialidase, a β-galactosidase, a β-glucuronidase, an α-galactosidase, or combinations thereof. In some embodiments, provided herein is a method of treating a disorder mediated by at least one N-linked glycan by administering to an individual (e.g., a human) in need thereof a therapeutically effective amount of an inhibitor of GlcNAc-TV, GlcNAc-TI, Glc-NAc-TII, or Glc-NAcTIV. In certain instances, the disorder mediated by an N-linked glycan is a cancer, a tumor, undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, organomegaly (e.g., hepatosplenomegaly), amyloidosis, skeletal abnormalities, odontoid hypoplasia, hydrops fetalis, inflammation, sialuria, sialidosis, thrombocytopenia, leukopenia, tumorous calcinosis, Ehlers-Danlos syndrome, Walker Warburg syndrome, a wound, or the like. In some embodiments, provided herein is a method of treating cancer by administering to an individual (e.g., a human) in need thereof a therapeutically effective amount of any N-linked glycan synthesis inhibitor described herein. In some embodiments, provided herein is a method of treating a tumor by administering to an individual (e.g., a human) in need thereof a therapeutically effective amount of any N-linked glycan synthesis inhibitor described herein. In some embodiments, provided herein is a method of treating undesired angiogenesis by administering to an individual (e.g., a human) in need thereof a therapeutically effective amount of any N-linked glycan synthesis inhibitor described herein. In some embodiments, provided herein is a method of treating a lysosomal storage disease (e.g., MPS) by administering to an individual (e.g., a human) in need thereof a therapeutically effective amount of any N-linked glycan synthesis inhibitor described herein. In some embodiments, provided herein is a method of treating a sialuria, sialidosis, thrombocytopenia, leukopenia, tumorous calcinosis and/or inflammation by administering to an individual (e.g., a human) in need thereof a therapeutically effective amount of any N-linked glycan synthesis inhibitor described herein. In some embodiments, provided herein is a method of treating cancer by administering to an individual (e.g., human) a therapeutically effective amount of any N-linked glycan synthesis inhibitor described herein. In some embodiments, the cancer is, by way of non-limiting example, pancreatic cancer, myeloma, ovarian cancer, hepatocellular cancer, breast cancer, colon carcinoma, renal cell carcinoma, carcinoma of the gut, lung or urogenital tract, or melanoma. In some embodiments, provided herein is a method of treating an infectious or viral disease by administering to an individual (e.g., human) a therapeutically effective amount of any N-linked glycan synthesis inhibitor described herein. In some embodiments, the infectious or viral disease includes, by way of non-limiting example, herpes, diphtheria, papilloma virus, hepatitis, HIV, coronavirus, or adenovirus.

In certain embodiments, provided herein is a method of treating a disorder mediated by N-linked glycans by administering to an individual (e.g., a human) in need thereof a therapeutically effective amount of any N-linked glycan synthesis inhibitor described herein. In some embodiments, the N-linked glycan synthesis inhibitor is a modulator (e.g., inhibitor or promoter) of a glycosyl transferase, an oligosaccharyl transferase, a mannosidase, a glucosidase, a phosphotransferase, a sialyl transferase, a fucosyl transferase, an acetyglucosaminyl transferase, an i-extension enzyme, a α sialidase, a β-galactosidase, a β-glucuronidase, an α-galactosidase, or combinations thereof. In some embodiments, provided herein is a method of treating a disorder mediated by at least one N-linked glycan by administering to an individual (e.g., a human) in need thereof a therapeutically effective amount of an inhibitor of GlcNAc-TV, GlcNAc-TI, Glc-NAc-TII, or Glc-NAcTIV. In certain instances, the disorder mediated by an N-linked glycan is an inflammatory disease (e.g, an acute or chronic inflammatory disorder) including but not limited to Crohn's disease, reactive arthritis, including Lyme disease, insulin-dependent diabetes, organ-specific auto immunity, Hashimoto's thyroiditis and Grave's disease, contact dermatitis, psoriasis, organ transplant rejection, graft rejection, graft versus host disease, sarcoidosis, atopic conditions, gastrointestinal allergies, including food allergies, pancreatitis, eosinophilia, conjunctivitis, glomerular nephritis, multiple vasculitides, myasthenia gravis, asthma, chronic obstructive pulmonary disease, myocardial infarction, stroke, transplant rejection, reperfusion injury, autoimmune disease (e.g, Ankylosing spondylitis, systemic lupus erythematosus (SLE), or the like) inflammatory bowel disease, psoriasis, arthritis (including, e.g., rheumatoid arthritis), allergic rhinitis, berillium disease, bronchiectasis, bronchitis, bronchopneumonia, cystic fibrosis, diphtheria, dyspnea, emphysema, allergic bronchopulmonary aspergillosis, pneumonia, acute pulmonary edema, pertussis, pharyngitis, atelectasis, Wegener's granulomatosis, Legionnaires disease, pleurisy, rheumatic fever, sinusitis or the like. In certain embodiments, the disorder mediated by N-linked glycan biosynthesis is a lysosomal storage disease (LSD) such as sialidosis (Type I, Type II) and fucosidosis. In some embodiments, the disorder mediated by N-linked glycan biosynthesis is an infectious disease targeting N-linked glycans of either host or the pathogen. In certain instances, this could affect the infection process or the pathogen life cycle. In various embodiments, pathogens may include bacteria, viruses (e.g., HIV, influenza, or the like) and fungi.

In some embodiments, an N-linked glycan biosynthesis inhibitor described herein is utilized as an adjuvant to enhance the immunogenicity of or the effectiveness of a vaccine. In certain instances, N-linked glycans may mask or otherwise alter certain epitopes. Thus, in some instances, inhibiting N-linked glycans may render epitopes more available or immunogenic during vaccine production or upon administration of the vaccine (e.g., gp120 gene from HIV).

Provided in certain embodiments herein is a process of inhibiting N-linked glycan function in a cell comprising contacting the cell with a selective modulator of N-linked glycan biosynthesis. In various embodiments, N-linked glycan biosynthesis, as used herein, includes, by way of non-limiting example, (1) inhibition of (a) a glycosyl transferase (e.g., an N-acetylgalactosaminyl transferase); (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporter that mediate N-linked glycan synthesis; and/or (2) promotion of (a) a glycosyl transferase; (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporters that mediate N-linked glycan synthesis. In some embodiments, the modulator of N-linked glycan biosynthesis inhibits the transfer of a N-acetylglucosaminyl moiety to a mannosyl moiety on N-linked glycans. In some embodiments, the modulator of N-linked glycan biosynthesis promotes the transfer of a N-acetylglucosaminyl moiety to a mannosyl moiety of N-linked glycans. In some embodiments, the modulator of N-linked glycan biosynthesis inhibits the linkage of a β1,6-N-acetylglucosaminyl moiety to a α1,6-mannosyl moiety on N-linked glycans. In some embodiments, the modulator of N-linked glycan biosynthesis promotes the transfer of a β1,6-N-acetylglucosaminyl moiety to a α1,6-mannosyl moiety of N-linked glycans. In some embodiments, the modulator of N-linked glycan biosynthesis inhibits the linkage of a β1,4-N-acetylglucosaminyl moiety to a α1,3-mannosyl moiety on N-linked glycans. In some embodiments, the modulator of N-linked glycan biosynthesis promotes the transfer of a β1,4-N-acetylglucosaminyl moiety to a α1,3-mannosyl moiety of N-linked glycans.

In some embodiments, the selective N-linked glycan synthesis inhibitor is a modulator of one or more of (e.g., promotes one or more of, or inhibits one or more of) synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α-1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), a transporter (e.g., a transporter for (Man α/β)₆-(GlcNAcβ)₂-Asn, one or more of the mannose residues (Man) being optionally phosphorylated).

In some embodiments, an N-linked glycan synthesis inhibitor modulates (e.g., promotes or inhibits) a glycosyltransferase (e.g., a UDP-GlcNAcT, GDP mannosyl transferase). In some embodiments, an inhibitor of a glycosyltransferase inhibits the synthesis of the precursor unit and/or the initiation of precursor unit synthesis. In some embodiments an N-linked glycan synthesis inhibitor modulates (e.g., promotes or inhibits) an oligosaccharyl transferase (e.g., D-OST). In some embodiments, an inhibitor of an oligosaccharyl transferase inhibits the attachment of a precursor unit to an Asn residue on a core protein. In some embodiments, an N-linked glycan synthesis inhibitor modulates (e.g., promotes or inhibits) a glycosidase (e.g., α1,2-glucosidase I, α1,3-glucosidase II, α-1,2-mannosidase, α1,2-specific Golgi mannosidase I, Golgi α1,6-mannosidase II, N-acetylglucosamine-1-phosphodiester alpha-N-acetylglucosaminidase). In some embodiments, an inhibitor of a glycosidase inhibits further processing (e.g., cleavage of residues) of a precursor unit attached to a core protein. In some embodiments, an N-linked glycan synthesis inhibitor modulates (e.g., promotes or inhibits) polymerization of a pentasaccharide core (e.g., promotes or inhibits GlcNAc-TI, GlcNAc-TII, GlcNAc-TIV, GlcNAc-TV, i-GnT, β-1,3-N-acetylglucosaminyltransferase, β-1,4-galactosyltransferase). In some embodiments, an N-linked glycan synthesis inhibitor modulates (e.g., promotes or inhibits) further modification of an N-linked glycan. In some embodiments, an inhibitor of further modification of a glycan inhibits, e.g., an i-extension enzyme, (e.g., iGnT), a polylactosamine extension enzyme, (e.g., β1-4-galactosyl transferase IV(β4Gal-TIV)) a fucosyl transferase (e.g., FucTVII, FucTIV), or a sialyl transferase (e.g., ST3GalIV, ST3GalVI), or a combination thereof.

In some instances, an N-linked glycan synthesis inhibitor alters or disrupts (e.g., synthesis of the β-1,6 branched N-linked glycans, e.g., synthesis of N-acetylglucosamine linked β-1,6- to an α1,3-mannose) the nature of the N-linked glycan such that it inhibits the binding, signaling, or a combination thereof of any protein subject to N-linked glycan binding, signaling or a combination thereof, compared to binding in the absence of an N-linked glycan synthesis inhibitor. In some instances, alteration or disruption of the nature of the N-linked glycan alters or modulates the presence of complex β-1,6-branched N-linked glycans in any protein subject to N-linked glycan binding, signaling or a combination thereof, compared to binding in the absence of an N-linked glycan synthesis inhibitor. In some instances, the alteration or modulation of the presence of complex β-1,6-branched N-linked glycans inhibits the binding, signaling, or a combination thereof of any protein subject to N-linked glycan and/or N-linked glycanated protein binding, signaling or a combination thereof, compared to binding in the absence of an N-linked glycan synthesis inhibitor.

In some instances, an N-linked glycan synthesis inhibitor alters or disrupts the nature of an N-linked glycan such that it inhibits the binding, signaling, or a combination thereof of any lectin (including polypeptides) subject to β-1,6-branched N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments the CAM is an exogenous CAM, e.g., bacterial lectins. In certain embodiments, the CAM is an endogenous CAM and includes, by way of non-limiting examples, E-selectin, L-selectin or P-selectin.

In some instances, an N-linked glycan synthesis inhibitor alters or disrupts the nature of an N-linked glycan such that it inhibits the binding, signaling, or a combination thereof of any lectin e.g., galectin-3 or any one or more galectin, (including polypeptides) subject to binding, signaling or a combination thereof to a β-1,6-branched N-linked glycan modified with N-acetyllactosamine, compared to binding in the absence of an N-linked glycan synthesis inhibitor. In some instances, an N-linked glycan synthesis inhibitor alters or disrupts the nature of an N-linked glycan such that it inhibits the binding, signaling, or a combination thereof of a protein, (e.g., integrin, matriptase and/or N-cadherin) subject to β-1,6-branched N-linked glycan binding, signaling or a combination thereof, compared to binding in the absence of an N-linked glycan synthesis inhibitor.

In certain embodiments, the selective modulator of N-linked glycan biosynthesis is a small molecule organic compound. In certain instances, selective modulator of N-linked glycan biosynthesis utilized herein is not a polypeptide or a carbohydrate. In certain embodiments, the small molecule organic compound has a molecular weight of less than about 2,000 g/mol, less than about 1,500 g/mol, less than about 1,000 g/mol, less than about 700 g/mol, or less than about 500 g/mol. In some embodiments, the N-linked glycan biosynthesis modulators are non-carbohydrate small molecules. In some embodiments, the N-linked glycan biosynthesis modulators are non-carbohydrate organic compounds. In some embodiments, the N-linked glycan biosynthesis modulators are non-carbohydrate small molecule organic compounds.

Provided in certain embodiments herein is a method of treating cancer or neoplasia comprising administering a therapeutically effective amount of an N-linked glycan biosynthesis inhibitor to a patient in need thereof. In some embodiments, the N-linked glycan biosynthesis inhibitor reduces or inhibits tumor growth, reduces or inhibits angiogenesis, or a combination thereof. In certain embodiments, the N-linked glycan biosynthesis inhibitor is a selective modulator of a glycosyl transferase, an oligosaccharyl transferase, a mannosidase, a glucosidase, a phosphotransferase, a sialyl transferase, a fucosyl transferase, an acetyglucosaminyl transferase, an i-extension enzyme, a α sialidase, a β-galactosidase, a β-glucuronidase, an α-galactosidase, or a combination thereof.

In various embodiments, N-linked glycan biosynthesis, as used herein, includes, by way of non-limiting example, (1) inhibition of (a) a glycosyl transferase (e.g., an N-acetylgalactosaminyl transferase); (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporter that mediate N-linked glycan synthesis; and/or (2) promotion of (a) a glycosyl transferase; (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporters that mediate N-linked glycan synthesis. In some embodiments, the modulator of N-linked glycan biosynthesis inhibits the transfer of a N-acetylglucosaminyl moiety to a mannosyl moiety on N-linked glycans. In some embodiments, the modulator of N-linked glycan biosynthesis promotes the transfer of a N-acetylglucosaminyl moiety to a mannosyl moiety of N-linked glycans. In some embodiments, the modulator of N-linked glycan biosynthesis inhibits the linkage of a β1,6-N-acetylglucosaminyl moiety to a α1,6-mannosyl moiety on N-linked glycans. In some embodiments, the modulator of N-linked glycan biosynthesis promotes the transfer of a β1,6-N-acetylglucosaminyl moiety to a α1,6-mannosyl moiety of N-linked glycans. In some embodiments, the modulator of N-linked glycan biosynthesis inhibits the linkage of a β1,4-N-acetylglucosaminyl moiety to a α1,3-mannosyl moiety on N-linked glycans. In some embodiments, the modulator of N-linked glycan biosynthesis promotes the transfer of a β1,4-N-acetylglucosaminyl moiety to a α1,3-mannosyl moiety of N-linked glycans.

In some embodiments, the selective N-linked glycan synthesis inhibitor is a modulator of one or more of (e.g., promotes one or more of, or inhibits one or more of) synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α-1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), a transporter (e.g., a transporter for (Man α/β)₆-(GlcNAcβ)₂-Asn, one or more of the mannose residues (Man) being optionally phosphorylated).

In some embodiments, an N-linked glycan synthesis inhibitor modulates (e.g., promotes or inhibits) a glycosyltransferase (e.g., a UDP-GlcNAcT, GDP mannosyl transferase). In some embodiments, an inhibitor of a glycosyltransferase inhibits the synthesis of the precursor unit and/or the initiation of precursor unit synthesis. In some embodiments an N-linked glycan synthesis inhibitor modulates (e.g., promotes or inhibits) an oligosaccharyl transferase (e.g., D-OST). In some embodiments, an inhibitor of an oligosaccharyl transferase inhibits the attachment of a precursor unit to an Asn residue on a core protein. In some embodiments, an N-linked glycan synthesis inhibitor modulates (e.g., promotes or inhibits) a glycosidase (e.g., α1,2-glucosidase I, α1,3-glucosidase II, α-1,2-mannosidase, α1,2-specific Golgi mannosidase I, Golgi α1,6-mannosidase II, N-acetylglucosamine-1-phosphodiester alpha-N-acetylglucosaminidase). In some embodiments, an inhibitor of a glycosidase inhibits further processing (e.g., cleavage of residues) of a precursor unit attached to a core protein. In some embodiments, an N-linked glycan synthesis inhibitor modulates (e.g., promotes or inhibits) polymerization of a pentasaccharide core (e.g., promotes or inhibits GlcNAc-TI, GlcNAc-TII, GlcNAc-TIV, GlcNAc-TV, i-GnT, β-1,3-N-acetylglucosaminyltransferase, β-1,4-galactosyltransferase). In some embodiments, an N-linked glycan synthesis inhibitor modulates (e.g., promotes or inhibits) further modification of an N-linked glycan. In some embodiments, an inhibitor of further modification of a glycan inhibits, e.g., an i-extension enzyme, (e.g., iGnT), a polylactosamine extension enzyme, (e.g., β1-4-galactosyl transferase IV(β4Gal-TIV)) a fucosyl transferase (e.g., FucTVII, FucTIV), or a sialyl transferase (e.g., ST3GalIV, ST3GalVI), or a combination thereof.

In some instances, an N-linked glycan synthesis inhibitor alters or disrupts (e.g., synthesis of the β-1,6 branched N-linked glycans, e.g., synthesis of N-acetylglucosamine linked β-1,6- to an α1,3-mannose) the nature of the N-linked glycan such that it inhibits the binding, signaling, or a combination thereof of any protein subject to N-linked glycan binding, signaling or a combination thereof, compared to binding in the absence of an N-linked glycan synthesis inhibitor. In some instances, alteration or disruption of the nature of the N-linked glycan alters or modulates the presence of complex β-1,6-branched N-linked glycans in any protein subject to N-linked glycan binding, signaling or a combination thereof, compared to binding in the absence of an N-linked glycan synthesis inhibitor. In some instances, the alteration or modulation of the presence of complex β-1,6-branched N-linked glycans inhibits the binding, signaling, or a combination thereof of any protein subject to N-linked glycan binding, signaling or a combination thereof, compared to binding in the absence of an N-linked glycan synthesis inhibitor.

In some instances, an N-linked glycan synthesis inhibitor alters or disrupts the nature of an N-linked glycan such that it inhibits the binding, signaling, or a combination thereof of any lectin (including polypeptides) subject to β-1,6-branched N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments the CAM is an exogenous CAM, e.g., bacterial lectins. In certain embodiments, the CAM is an endogenous CAM and includes, by way of non-limiting examples, E-selectin, L-selectin or P-selectin.

In some instances, an N-linked glycan synthesis inhibitor alters or disrupts the nature of an N-linked glycan such that it inhibits the binding, signaling, or a combination thereof of any lectin e.g., galectin-3 or any one or more galectin, (including polypeptides) subject to binding, signaling or a combination thereof to a β-1,6-branched N-linked glycan modified with N-acetyllactosamine, compared to binding in the absence of an N-linked glycan synthesis inhibitor. In some instances, an N-linked glycan synthesis inhibitor alters or disrupts the nature of an N-linked glycan such that it inhibits the binding, signaling, or a combination thereof of a protein, (e.g., integrin, matriptase and/or N-cadherin) subject to β-1,6-branched N-linked glycan binding, signaling or a combination thereof, compared to binding in the absence of an N-linked glycan synthesis inhibitor.

In certain embodiments, N-linked glycan synthesis inhibitors described herein are small molecule organic compounds. In certain instances, N-linked glycan synthesis utilized herein are not polypeptides or carbohydrates. In some embodiments, a small molecule organic compounds has a molecular weight of less than about 2,000 g/mol, less than about 1,500 g/mol, less than about 1,000 g/mol, less than about 700 g/mol, or less than about 500 g/mol. In some embodiments, the N-linked glycan synthesis inhibitors are non-carbohydrate small molecules. In some embodiments, the N-linked glycan synthesis inhibitors are non-carbohydrate organic compounds. In some embodiments, the N-linked glycan synthesis inhibitors are non-carbohydrate small molecule organic compounds.

Provided in some embodiments herein is a method of treating a lysosomal storage disease comprising administering a therapeutically effective amount of an N-linked glycan biosynthesis inhibitor to an individual (e.g., a human) in need thereof. In certain embodiments, the N-linked glycan synthesis modulator is an endoglycosidase and/or an exoglycosidase. In some embodiments, modulation of endoglycosidases and/or exoglycosidases includes the promotion and/or inhibition of β-N-acetylhexosaminidase (e.g. promotion and/or inhibition of βGlcNAc and/or βGalNAc), sialidase (e.g. neuraminidase), glycosylasparginase, β-galactosidase, β-glucuronidase, α-galactosidase or Cathepsin A. In some embodiments, the N-linked glycan synthesis modulator is a selective inhibitor of any glycosyl transferase, oligosaccharyl transferase, mannosidase, glucosidase, phosphotransferase, sialyl transferase, a fucosyl transferase, acetyglucosaminyl transferase, i-extension enzyme, α sialidase, β-galactosidase, β-glucuronidase, α-galactosidase described herein, or a combination thereof. In some embodiments, the lysosomal storage disease is, by way of non-limiting example, mucopolysaccharidosis (MPS). In more embodiments, the MPS is, by way of non-limiting example, MPS I, MPS II or MPS III.

In various embodiments, N-linked glycan biosynthesis, as used herein, includes, by way of non-limiting example, (1) inhibition of (a) a glycosyl transferase (e.g., an N-acetylgalactosaminyl transferase); (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporter that mediate N-linked glycan synthesis; and/or (2) promotion of (a) a glycosyl transferase; (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporters that mediate N-linked glycan synthesis. In some embodiments, the modulator of N-linked glycan biosynthesis inhibits the transfer of a N-acetylglucosaminyl moiety to a mannosyl moiety on N-linked glycans. In some embodiments, the modulator of N-linked glycan biosynthesis promotes the transfer of a N-acetylglucosaminyl moiety to a mannosyl moiety of N-linked glycans. In some embodiments, the modulator of N-linked glycan biosynthesis inhibits the linkage of a β1,6-N-acetylglucosaminyl moiety to a α1,6-mannosyl moiety on N-linked glycans. In some embodiments, the modulator of N-linked glycan biosynthesis promotes the transfer of a β1,6-N-acetylglucosaminyl moiety to a α1,6-mannosyl moiety of N-linked glycans. In some embodiments, the modulator of N-linked glycan biosynthesis inhibits the linkage of a β1,4-N-acetylglucosaminyl moiety to a α1,3-mannosyl moiety on N-linked glycans. In some embodiments, the modulator of N-linked glycan biosynthesis promotes the transfer of a β1,4-N-acetylglucosaminyl moiety to a α1,3-mannosyl moiety of N-linked glycans.

In some embodiments, the selective N-linked glycan synthesis inhibitor is a modulator of one or more of (e.g., promotes one or more of, or inhibits one or more of) synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α-1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), a transporter (e.g., a transporter for (Man α/β)₆-(GlcNAcβ)₂-Asn, one or more of the mannose residues (Man) being optionally phosphorylated).

In some embodiments, an N-linked glycan synthesis inhibitor modulates (e.g., promotes or inhibits) a glycosyltransferase (e.g., a UDP-GlcNAcT, GDP mannosyl transferase). In some embodiments, an inhibitor of a glycosyltransferase inhibits the synthesis of the precursor unit and/or the initiation of precursor unit synthesis. In some embodiments an N-linked glycan synthesis inhibitor modulates (e.g., promotes or inhibits) an oligosaccharyl transferase (e.g., D-OST). In some embodiments, an inhibitor of an oligosaccharyl transferase inhibits the attachment of a precursor unit to an Asn residue on a core protein. In some embodiments, an N-linked glycan synthesis inhibitor modulates (e.g., promotes or inhibits) a glycosidase (e.g., α1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase). In some embodiments, an inhibitor of a glycosidase inhibits further processing (e.g., cleavage of residues) of a precursor unit attached to a core protein. In some embodiments, an N-linked glycan synthesis inhibitor modulates (e.g., promotes or inhibits) polymerization of a pentasaccharide core (e.g., promotes or inhibits GlcNAc-TI, GlcNAc-TII, GlcNAc-TIV, GlcNAc-TV, i-GnT, β-1,3-N-acetylglucosaminyltransferase, β-1,4-galactosyltransferase). In some embodiments, an N-linked glycan synthesis inhibitor modulates (e.g., promotes or inhibits) further modification of an N-linked glycan. In some embodiments, an inhibitor of further modification of a glycan inhibits, e.g., an i-extension enzyme, (e.g., iGnT), a polylactosamine extension enzyme, (e.g., β1-4-galactosyltransferase IV(β4Gal-TIV)) a fucosyl transferase (e.g., FucTVII, FucTIV), or a sialyl transferase (e.g., ST3Ga11V, ST3GalVI), or a combination thereof.

In some instances, an N-linked glycan synthesis inhibitor alters or disrupts (e.g., synthesis of the β-1,6 branched N-linked glycans, e.g., synthesis of N-acetylglucosamine linked β-1,6- to an α1,3-mannose) the nature of the N-linked glycan such that it inhibits the binding, signaling, or a combination thereof of any protein subject to N-linked glycan binding, signaling or a combination thereof, compared to binding in the absence of an N-linked glycan synthesis inhibitor. In some instances, alteration or disruption of the nature of the N-linked glycan alters or modulates the presence of complex β-1,6-branched N-linked glycans in any protein subject to N-linked glycan binding, signaling or a combination thereof, compared to binding in the absence of an N-linked glycan synthesis inhibitor. In some instances, the alteration or modulation of the presence of complex β-1,6-branched N-linked glycans inhibits the binding, signaling, or a combination thereof of any protein subject to N-linked glycan binding, signaling or a combination thereof, compared to binding in the absence of an N-linked glycan synthesis inhibitor.

In some instances, an N-linked glycan synthesis inhibitor alters or disrupts the nature of an N-linked glycan such that it inhibits the binding, signaling, or a combination thereof of any lectin (including polypeptides) subject to β-1,6-branched N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments the CAM is an exogenous CAM, e.g., bacterial lectins. In certain embodiments, the CAM is an endogenous CAM and includes, by way of non-limiting examples, E-selectin, L-selectin or P-selectin.

In some instances, an N-linked glycan synthesis inhibitor alters or disrupts the nature of an N-linked glycan such that it inhibits the binding, signaling, or a combination thereof of any lectin e.g., galectin-3 or any one or more galectin, (including polypeptides) subject to binding, signaling or a combination thereof to a β-1,6-branched N-linked glycan modified with N-acetyllactosamine, compared to binding in the absence of an N-linked glycan synthesis inhibitor. In some instances, an N-linked glycan synthesis inhibitor alters or disrupts the nature of an N-linked glycan such that it inhibits the binding, signaling, or a combination thereof of a protein, (e.g., integrin, matriptase and/or N-cadherin) subject to β-1,6-branched N-linked glycan binding, signaling or a combination thereof, compared to binding in the absence of an N-linked glycan synthesis inhibitor.

In certain embodiments, N-linked glycan synthesis inhibitors described herein are small molecule organic compounds. In certain instances, N-linked glycan synthesis inhibitors utilized herein are not polypeptides or carbohydrates. In some embodiments, a small molecule organic compounds has a molecular weight of less than about 2,000 g/mol, less than about 1,500 g/mol, less than about 1,000 g/mol, less than about 700 g/mol, or less than about 500 g/mol. In some embodiments, the N-linked glycan synthesis inhibitors are non-carbohydrate small molecules. In some embodiments, the N-linked glycan synthesis inhibitors are non-carbohydrate organic compounds. In some embodiments, the N-linked glycan synthesis inhibitors are non-carbohydrate small molecule organic compounds.

Provided in certain embodiments herein is a method of reducing the mean or median number of β1,6-branched N-acetylglucosaminyl residues of a N-linked glycan in (or endogenous to) an individual comprising administering a therapeutically effective amount of an N-linked glycan synthesis inhibitor to an individual in need thereof. In certain embodiments, the method of reducing the mean or median number of β1,6-branched N-acetylglucosaminyl residues of a N-linked glycan in (or endogenous to) an individual comprising administering a therapeutically effective amount of an N-linked glycan synthesis inhibitor to an individual in need thereof is suitable for treating cancer or the symptoms thereof. In certain embodiments, the N-linked glycan synthesis is a selective inhibitor of a glycosyl transferase, an oligosaccharyl transferase, a mannosidase, a glucosidase, a phosphotransferase, a sialyl transferase, a fucosyl transferase, an acetyglucosaminyl transferase, an i-extension enzyme, a α sialidase, a β-galactosidase, a β-glucuronidase, an α-galactosidase, or a combination thereof. In some embodiments, the cancer is by way of example, a carcinoma, or an adenocarcinoma.

In various embodiments, N-linked glycan biosynthesis, as used herein, includes, by way of non-limiting example, (1) inhibition of (a) a glycosyl transferase (e.g., an N-acetylglucosaminyl transferase); (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporter that mediate N-linked glycan synthesis; and/or (2) promotion of (a) a glycosyl transferase; (b) oligosaccharyl transferase (c) modification of a precursor unit to a pentasaccharide unit (d) polymerization of the pentasaccharide unit (e) fucosylation; (f) sialylation (g) phosphorylation and/or (i) chaperones and/or transporters that mediate N-linked glycan synthesis. In some embodiments, the modulator of N-linked glycan biosynthesis inhibits the transfer of a N-acetylglucosaminyl moiety to a mannosyl moiety on N-linked glycans. In some embodiments, the modulator of N-linked glycan biosynthesis promotes the transfer of a N-acetylglucosaminyl moiety to a mannosyl moiety of N-linked glycans. In some embodiments, the modulator of N-linked glycan biosynthesis inhibits the linkage of a β1,6-N-acetylglucosaminyl moiety to a α1,6-mannosyl moiety on N-linked glycans. In some embodiments, the modulator of N-linked glycan biosynthesis promotes the transfer of a β1,6-N-acetylglucosaminyl moiety to a α1,6-mannosyl moiety of N-linked glycans. In some embodiments, the modulator of N-linked glycan biosynthesis inhibits the linkage of a β1,4-N-acetylglucosaminyl moiety to a α1,3-mannosyl moiety on N-linked glycans. In some embodiments, the modulator of N-linked glycan biosynthesis promotes the transfer of a β1,4-N-acetylglucosaminyl moiety to a α1,3-mannosyl moiety of N-linked glycans.

In some embodiments, the selective N-linked glycan synthesis inhibitor is a modulator of one or more of (e.g., promotes one or more of, or inhibits one or more of) synthesis of a precursor unit (e.g., modulates a UDP-GlcNAcT, GDP mannosyl transferase), attachment of a precursor unit to an Asn residue on a protein (e.g. modulates Dolichol-OST), further processing (e.g., cleavage of residues) of a precursor unit (e.g., modulates α-1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase), glycophosphorylation (e.g., modulates N-acetylglucosaminylphosphotransferase), further polymerization of the pentasaccharide core (e.g., modulates a GlcNAc-TI, GlcNAc-TII, GlcNAc-TIV, GlcNAc-TV, i-GnT, β-1,3-N-acetylglucosaminyltransferase, β-1,4-galactosyltransferase), further modification of N-linked glycan, e.g., sialylation (e.g., modulates a sialyl transferase), fucosylation (e.g., modulates a fucosyl transferase), a transporter (e.g., a transporter for (Man α/β)₆-(GlcNAcβ)₂-Asn, one or more of the mannose residues (Man) being optionally phosphorylated).

In some embodiments, an N-linked glycan synthesis inhibitor modulates (e.g., promotes or inhibits) a glycosyltransferase (e.g., a UDP-GlcNAcT, GDP mannosyl transferase). In some embodiments, an inhibitor of a glycosyltransferase inhibits the synthesis of the precursor unit and/or the initiation of precursor unit synthesis. In some embodiments an N-linked glycan synthesis inhibitor modulates (e.g., promotes or inhibits) an oligosaccharyl transferase (e.g., D-OST). In some embodiments, an inhibitor of an oligosaccharyl transferase inhibits the attachment of a precursor unit to an Asn residue on a core protein. In some embodiments, an N-linked glycan synthesis inhibitor modulates (e.g., promotes or inhibits) a glycosidase (e.g., α1,2-glucosidase I, α1,3-glucosidase II, α1,2-mannosidase, α1,2-specific Golgi mannosidase I, Golgi α1,6-mannosidase II, N-acetylglucosamine-1-phosphodiester alpha-N-acetylglucosaminidase). In some embodiments, an inhibitor of a glycosidase inhibits further processing (e.g., cleavage of residues) of a precursor unit attached to a core protein. In some embodiments, an N-linked glycan synthesis inhibitor modulates (e.g., promotes or inhibits) polymerization of a pentasaccharide core (e.g., promotes or inhibits GlcNAc-TI, GlcNAc-TII, GlcNAc-TIV, GlcNAc-TV, i-GnT, β-1,3-N-acetylglucosaminyltransferase, β-1,4-galactosyltransferase). In some embodiments, an N-linked glycan synthesis inhibitor modulates (e.g., promotes or inhibits) further modification of an N-linked glycan. In some embodiments, an inhibitor of further modification of a glycan inhibits, e.g., an i-extension enzyme, (e.g., iGnT), a polylactosamine extension enzyme, (e.g., β1-4-galactosyl transferase IV(β4Gal-TIV)) a fucosyl transferase (e.g., FucTVII, FucTIV), or a sialyl transferase (e.g., ST3GalIV, ST3GalVI), or a combination thereof.

In some instances, an N-linked glycan synthesis inhibitor alters or disrupts (e.g., synthesis of the β-1,6 branched N-linked glycans, e.g., synthesis of N-acetylglucosamine linked β-1,6- to an α1,3-mannose) the nature of the N-linked glycan such that it inhibits the binding, signaling, or a combination thereof of any protein subject to N-linked glycan binding, signaling or a combination thereof, compared to binding in the absence of an N-linked glycan synthesis inhibitor. In some instances, alteration or disruption of the nature of the N-linked glycan alters or modulates the presence of complex β-1,6-branched N-linked glycans in any protein subject to N-linked glycan binding, signaling or a combination thereof, compared to binding in the absence of an N-linked glycan synthesis inhibitor. In some instances, the alteration or modulation of the presence of complex β-1,6-branched N-linked glycans inhibits the binding, signaling, or a combination thereof of any protein subject to N-linked glycan binding, signaling or a combination thereof, compared to binding in the absence of an N-linked glycan synthesis inhibitor.

In some instances, an N-linked glycan synthesis inhibitor alters or disrupts the nature of an N-linked glycan such that it inhibits the binding, signaling, or a combination thereof of any lectin (including polypeptides) subject to β-1,6-branched N-linked glycan binding, signaling or a combination thereof, compared to binding in the absence of an N-linked glycan synthesis inhibitor. In some instances, the polypeptide is, by way of non-limiting example, a cell adhesion molecule (CAM). In certain embodiments the CAM is an exogenous CAM, e.g., bacterial lectins. In certain embodiments, the CAM is an endogenous CAM and includes, by way of non-limiting examples, E-selectin, L-selectin or P-selectin.

In some instances, an N-linked glycan synthesis inhibitor alters or disrupts the nature of an N-linked glycan such that it inhibits the binding, signaling, or a combination thereof of any lectin e.g., galectin-3 or any one or more galectin, (including polypeptides) subject to binding, signaling or a combination thereof to a β-1,6-branched N-linked glycan modified with N-acetyllactosamine, compared to binding in the absence of an N-linked glycan synthesis inhibitor. In some instances, an N-linked glycan synthesis inhibitor alters or disrupts the nature of an N-linked glycan such that it inhibits the binding, signaling, or a combination thereof of a protein, (e.g., integrin, matriptase and/or N-cadherin) subject to β-1,6-branched N-linked glycan binding, signaling or a combination thereof, compared to binding in the absence of an N-linked glycan synthesis inhibitor.

In certain embodiments, N-linked glycan synthesis inhibitors described herein are small molecule organic compounds. In certain instances, N-linked glycan synthesis inhibitors utilized herein are not polypeptides or carbohydrates. In some embodiments, a small molecule organic compounds has a molecular weight of less than about 2,000 g/mol, less than about 1,500 g/mol, less than about 1,000 g/mol, less than about 700 g/mol, or less than about 500 g/mol. In some embodiments, the N-linked glycan synthesis inhibitors are non-carbohydrate small molecules. In some embodiments, the N-linked glycan synthesis inhibitors are non-carbohydrate organic compounds. In some embodiments, the N-linked glycan synthesis inhibitors are non-carbohydrate small molecule organic compounds.

### Screening Processes

Provided herein are processes for identifying inhibitors of the biosynthesis of N-linked glycans or for identifying genes involved in (including selective regulators of) the biosynthesis of N-linked glycans. Also provided herein are processes for identifying modulators of enzymes involved in the biosynthesis of N-linked glycans.

In one embodiment is a cell-based high throughput process for identifying and/or screening for (1) N-linked glycan biosynthesis inhibitors; (2) genes involved in (including selective regulators of) the biosynthesis of N-linked glycans; (3) N-linked glycan biosynthesis modulators; or (4) combinations thereof. In one embodiment, a library of small-molecule chemical compounds (including oligopeptides and oligonucleotides) is screened; in other embodiments, a library of siRNA is screened; in other embodiments, both types of libraries are simultaneously or sequentially screened.

In certain embodiments, the siRNA library is enzymatically generated; or rationally synthesized; or randomly generated; or a combination thereof. Non-limiting examples of protocols for screening siRNA libraries in high-throughput genetic screens is found in the Journal of Cancer Molecules: 1(1), 19-24, 2005.

Provided in some embodiments is a process for identifying a compound that modulates N-linked glycan biosynthesis comprising:
a. contacting a mammalian cell with the compound in combination with a labeled probe that binds one or more N-linked glycans;
b. incubating the mammalian cell, compound and labeled probe;
c. collecting the labeled probe that is bound to one or more N-linked glycans; and
d. detecting or measuring the amount of labeled probe bound to one or more N-linked glycans.

In more specific embodiments, provided herein is a process for identifying a compound that selectively modulates N-glycan biosynthesis comprising:
a. contacting a mammalian cell with the compound
b. contacting the mammalian cell and compound combination with a first labeled probe and a second labeled probe, wherein the first labeled probe binds one or more N-linked glycans and the second labeled probe binds at least one glycan (e.g., a GAG, a sulfated GAG, an extracellular glycan, or the like) other than N-linked glycans;
c. incubating the mammalian cell, compound, the first labeled probe, and the second labeled probe;
d. collecting the first labeled probe that is bound to one or more N-linked glycans;
e. collecting the second labeled probe that is bound to at least one glycan (e.g., a GAG, a sulfated GAG, an extracellular glycan, or the like) other than N-linked glycans;
f. detecting or measuring the amount of first labeled probe bound to one or more N-linked glycans; and
g. detecting or measuring the amount of the second labeled probe bound to at least one glycan (e.g., a GAG, a sulfated GAG, an extracellular glycan, or the like) other than N-linked glycans.

Similarly, in some embodiments provided herein is a process for identifying compounds that selectively modulate N-linked glycans biosynthesis comprising:
a. contacting a first mammalian cell with the compound
b. contacting the first mammalian cell and compound combination with a first labeled probe, wherein the first labeled probe binds one or more N-linked glycans;
c. incubating the first mammalian cell, compound, the first labeled probe, and the second labeled probe;
d. collecting the first labeled probe that is bound to one or more N-linked glycans;
e. detecting or measuring the amount of first labeled probe bound to one or more N-linked glycans;
f. contacting a second mammalian cell with the compound, wherein the second mammalian cell is of the same type as the first mammalian cell;
g. contacting the second mammalian cell and compound combination with a second labeled probe, wherein the second labeled probe binds at least one glycan (e.g., a GAG, a sulfated GAG, an extracellular glycan, or the like) other than N-linked glycans;
h. collecting the second labeled probe that is bound to at least one glycan (e.g., a GAG, a sulfated GAG, an extracellular glycan, or the like) other than N-linked glycans; and
i. detecting or measuring the amount of the second labeled probe bound to at least one glycan (e.g., a GAG, a sulfated GAG, an extracellular glycan, or the like) other than N-linked glycans.

In some embodiments, provided herein is a process for identifying a compound that modulates N-linked glycan biosynthesis comprising:
a. collecting N-linked glycans from a first mammalian cell of a selected type, wherein the N-linked glycan comprises a plurality of high mannose, hybrid or complex N-linked glycan structures;
b. cleaving the N-linked glycans into a plurality of monosaccharide, disaccharide or oligosaccharide component parts;
c. detecting or measuring the amount of one or more of the monosaccharide, disaccharide or oligosaccharide component parts;
d. contacting and incubating a second mammalian cell of the selected type with the compound;
e. collecting N-linked glycans from the second mammalian cell of a selected type;
f. cleaving the N-linked glycans into a plurality of monosaccharide, disaccharide or oligosaccharide component parts;
g. detecting or measuring the amount of one or more of the monosaccharide, disaccharide or oligosaccharide component parts;
h. comparing:
   i. the amounts of N-linked glycans, or one or more of the monosaccharide, disaccharide or oligosaccharide component parts thereof, produced by the first and second mammalian cells;
   ii. the amounts of monosaccharide, disaccharide or oligosaccharide component parts characteristic of di-antennary N-linked glycans, tri-antennary N-linked glycans or tetra-antennary N-linked glycans present in the N-linked glycans;
   iii. the relative amounts of monosaccharide, disaccharide or oligosaccharide component parts characteristic of di-antennary N-linked glycans, tri-antennary N-linked glycans or tetra-antennary N-linked glycans present in the N-linked glycans; or
   iv. a combination thereof.

In some embodiments, monosaccharide, disaccharide or oligosaccharide component parts characteristic of N-linked glycans are monosaccharide, disaccharide or oligosaccharide component parts of di-antennary N-linked glycans, tri-antennary N-linked glycans and/or tetra-antennary N-linked glycans. In some embodiments, monosaccharide, disaccharide or oligosaccharide component parts of di-antennary N-linked glycans, tri-antennary N-linked glycans and/or tetra-antennary N-linked glycans are mannosyl residues and/or sialyl residues. In some embodiments, the amount of any specific di-antennary N-linked glycan, tri-antennary N-linked glycan and/or tetra-antennary N-linked glycan collected from a first mammalian cell is compared to the amount of any other specific type of di-antennary N-linked glycan, tri-antennary N-linked glycan or tetra-antennary N-linked glycan collected from a second mammalian cell. In some embodiments, the amounts of one or more specific di-antennary N-linked glycans, tri-antennary N-linked glycans and/or tetra-antennary N-linked glycans collected from a first mammalian cell are compared to the amounts of one or more of any other specific type of di-antennary N-linked glycan, tri-antennary N-linked glycan or tetra-antennary N-linked glycans or the total amount of N-linked glycans collected from a second mammalian cell.

In some embodiments, incubating the mixture of the compound with the at least one cell expressing at least one N-linked glycan is performed for a predetermined time. In one embodiment, incubation is for a period of about 12 hours. In another embodiment, incubating the mixture is for a period of about 18 hours. In another embodiment, about 24 hours. In yet another embodiment, about 36 hours. In a further embodiment, 48 hours. In another embodiment, at least about 12 hours, at least about 24 hours, at least about 36 hours, at least about 2 days, at least about 3 days, at least about 4 days, at least about 5 days, at least about 6 days, or at least about 7 days.

In one embodiment, the process(es) described herein are useful for high-throughput analysis of an N-linked glycan biosynthesis inhibitor or a positive or negative regulatory gene for N-linked glycan biosynthesis. In one embodiment the positive or negative regulatory gene of N-linked N-acetylglucosaminyl transferase is a positive or negative regulatory gene of N-acetylglucosaminyl transferase V. In another embodiment the at least one cell expressing at least one N-linked glycan is a Chinese hamster ovary cell (CHO) or a human tumor cell. In yet another embodiment the human tumor cell is selected from HeLa, LS-180, PC-3, MeWo, and HT29 cells.

In certain embodiments, the amounts of N-linked glycans and/or monosaccharides, disaccharides or oligosaccharides characteristic of N-linked glycans are measured with an analytical device. In some embodiments, the analytical device is a fluorimeter. In some embodiments, the analytical device is a fluorescent plate reader. In some embodiments, fluorescence is measured at any suitable excitation (e.g., an excitation of about 400-600 nm) and any suitable emission (e.g., about 500-750 nm). In some embodiments, the detecting or measuring process is developed using a robotic pipettor.

In one embodiment, the inhibitor of N-linked glycan biosynthesis is an inhibitor of mannosidase, an N-linked glycan N-acetylglucosaminyl transferase, an N-linked glycan fucosyl transferase, an N-linked glycan galactosyl transferase, an N-linked glycan sialyl transferase, an N-linked glycan sulfotransferase, or N-linked glycan glycophosphotransferase or a combination thereof.

In some embodiments, the process further comprises comparing the amount of first labeled probe bound to one or more N-linked glycans to the amount of the second labeled probe bound to at least one glycan other than N-linked glycans (e.g., to determine a ratio of the amount of first labeled probe bound to the amount of second labeled probe bound under substantially similar conditions).

In certain embodiments, a label utilized in any process described herein is any suitable label such as, by way of non-limiting example, a fluorescent label, a dye, a radiolabel, or the like. In some embodiments, the labeled probe comprises a biotinyl moiety and the process further comprises tagging the labeled probe with streptavidin-Cy5-PE. In certain embodiments, the first probe is any N-linked glycan binding protein, e.g., PHA. In various embodiments, the amount of bound labeled probes are detected in any suitable manner, e.g., with a fluorimeter, a radiation detector, or the like.

In certain embodiments, the first and second probes are labeled in a manner so as to be independently detectable. In some embodiments, the first and second probes are contacted to the cells separately (i.e., to different cells of the same type) and independently analyzed. In some embodiments, the at least one glycan (e.g., a GAG, a sulfated GAG, an extracellular glycan, or the like) other than N-linked glycans is, by way of non-limiting example, chondroitin sulfate, gangliosides, O-glycans, heparan sulfate or the like. Furthermore, in some embodiments, a third labeled probe that binds at least one glycan (e.g., a GAG, a sulfated GAG, an extracellular glycan, or the like) not bound by the first or second labeled probe is also utilized. Additional labeled probes are also optionally utilized.

Second and additional labeled probes include any labeled compound or labeled lectin suitable (e.g., a labeled compound or lectin that binds a ganglioside, a GAG, a non-sulfated GAG, an extracellular glycan, an O-linked glycan, chondroitin sulfate, dermatan sulfate, heparan sulfate, keratin sulfate, and/or hyaluronan). In some embodiments, labeled probes included labeled forms of one or more of, by way of non-limiting example, Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); Cholera Toxin B-subunit (CTB) from Vibrio cholera (as a probe for binding sialic acid modified glycolipids); Concanavalin A (ConA) from Canavalia ensiformis (as a probe for binding mannose residues in N-linked glycans); and/or Jacalin from Artocarpus integrifolia (as a probe for binding O-linked glycans). In specific embodiments, labeled forms of each of Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); and Cholera Toxin B-subunit (CTB) from Vibrio cholera (as a probe for binding sialic acid modified glycolipids) are utilized.

Contact with first, second and additional labeled probes occurs in parallel, concurrently, or sequentially. In certain embodiments, contact the compounds and multiple probes allows identification of selective N-linked glycan inhibitors.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)), a human ovarian cancer cell (SKOV), a human lung cancer cell (Ha18), a human meduloblastoma cancer cell (DAOY), a Chinese Hamster Ovary (CHO) cell, an adenocarcinoma cell, a melanoma cell, or a human primary cell. In certain embodiments, included herein are processes wherein the cell includes a plurality (e.g., 2, 3, 4 or all) of a human cancer cell (e.g., human cervical cancer cell (HeLa)), a human ovarian cancer cell (SKOV), a human lung cancer cell (Ha18), a human meduloblastoma cancer cell (DAOY), and/or a Chinese Hamster Ovary (CHO) cell. Contact with such cells optionally occurs in parallel, concurrently, or sequentially. In certain embodiments, contact with multiple cells identifies inhibitors (e.g., selective N-linked glycan synthesis inhibitors) that inhibit N-linked glycan biosynthesis in multiple cell lines. In some instances, utilization of a plurality of cell lines allows the elimination or minimization of false positives in identifying N-linked glycan inhibitors.

Thus, in some embodiments, any process described herein comprises contacting the compound to a first cell (type), contacting the compound to a second cell (type), and, optionally, contacting the compound to additional cells (types), and repeating the process described for each of the first, second and any additional cell types utilized (e.g., to determine if a N-linked glycan inhibitor is selective for multiple cell lines or to determine which types of cell lines that the N-linked glycan inhibitor selectively targets). Furthermore, in such embodiments, the process further comprises comparing the amount of labeled probe (or the amount of first, second or any additional labeled probe) that is bound in each type of cell (e.g., to determine selectively of inhibiting N-linked glycan biosynthesis compared to the biosynthesis of other types of glycans).

In some embodiments, once a compound that modulates N-linked glycan biosynthesis is determined by the process described, a similar process is optionally utilized to determine whether or not the compound selectively modulates N-linked glycan biosynthesis. Specifically, selectivity of a compound that modulates N-linked glycan biosynthesis is determined by utilizing a similar process as described for determining whether or not the compound modulates N-linked glycan biosynthesis, e.g., by:
a. contacting a mammalian cell with the compound in combination with a labeled probe that binds one or more non-N-linked glycan (e.g., GAG or other class of glycan);
b. incubating the mammalian cell, compound and labeled probe;
c. collecting the labeled probe that is bound to non-N-linked glycan (e.g., GAG or other class of glycan); and
d. detecting or measuring the amount of labeled probe bound to non-N-linked glycan (e.g., GAG or other class of glycan).

In various embodiments, this process is repeated for any number of non-N-linked glycans (e.g., GAG or other class of glycan). In some embodiments, the non-N-linked glycans are, by way of non-limiting example, chondroitin sulfate, heparan sulfate, O-linked glycans, gangliosides, or the like.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)) a human ovarian cancer cell (SKOV), a human lung cancer cell (Ha18), a human meduloblastoma cancer cell (DAOY) or a human primary cell. Furthermore, in some embodiments, the process is repeated utilizing one or more additional cell types. In certain embodiments, the results (e.g., of (c), and/or (d)) from the one or more additional cell types (e.g., a second, third, fourth, fifth or the like cell types) are compared to each other and the results (e.g., of (c), and/or (d)) from the first cell type.

In certain embodiments, the N-linked glycans and/or the modified N-linked glycans are cleaved in any suitable manner. In some embodiments, the N-linked glycans and/or the modified O-glycans are cleaved using a suitable enzyme such as PNGase-F, or in any other suitable chemical manner.

In some embodiments, the amount of monosaccharide, disaccharide or oligosaccharide units present in the cell and/or the characteristic of the N-linked glycans in a cell are determined in any suitable manner. For example, in some embodiments, the amount of sialyl and/or fucosyl and/or mannosyl units present and/or the amount of O-sulfation (e.g., 3-O-sulfation) of the glucosylamine groups, or a combination thereof is determined utilizing a carbozole assay, high performance liquid chromatography (HPLC), Thin layer chromatography (TLC), capillary electrophoresis, gel electrophoresis, mass spectrum (MS) analysis, HPLC electrospray ionization tandem mass spectrometry, nuclear magnetic resonance (NMR) analysis, or the like.

Moreover, in certain embodiments, the process described is a process for identifying compounds that selectively modulate N-linked glycan biosynthesis. In such embodiments, the process also comprises collecting one or more non-N-linked glycan (e.g., a sulfated glycan, such as chondroitin sulfate, O-linked glycans, or the like) from the cell, both without incubation with the compound and with incubation with the compound; cleaving each of such non-N-linked glycans; measuring the character of each of such non-N-linked glycan; and comparing the character of the non-N-linked glycan that was not incubated with the character of the non-N-linked glycan that was incubated. In certain embodiments, the character includes, by way of non-limiting example, the chain length of the non-N-linked glycan, the amount of sulfation of the non-N-linked glycan, the location of sulfation of the non-N-linked glycan, the structure of the non-N-linked glycan, the composition of the non-N-linked glycan, or the like. The structure of glycosaminoglycans, N-linked glycans, O-linked glycans, and lipid linked glycans can be determined using any suitable method, including, by way of non-limiting example, monosaccharide compositional analysis, capillary electrophoresis, gel electrophoresis, gel filtration, high performance liquid chromatography (HPLC), thin layer chromatography (TLC), mass spectrum (MS) analysis, HPLC electrospray ionization tandem mass spectrometry, nuclear magnetic resonance (NMR) analysis, or the like.

### Combinations

In certain instances, it is appropriate to administer at least one therapeutic compound described herein (i.e., any N-linked glycan inhibitor described herein) in combination with another therapeutic agent. By way of example only, if one of the side effects experienced by a patient upon receiving one of the N-linked glycan inhibitors described herein is nausea, then it is appropriate in certain instances to administer an anti-nausea agent in combination with the initial therapeutic agent. Or, by way of example only, the therapeutic effectiveness of one of the N-linked glycan inhibitors described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering one of N-linked glycan inhibitors described herein with another therapeutic agent (which also includes a therapeutic regimen) that also has therapeutic benefit. In any case, regardless of the disease, disorder or condition being treated, the overall benefit experienced by the patient is in some embodiments additive of the two therapeutic agents or in other embodiments, the patient experiences a synergistic benefit.

In some embodiments, the particular choice of compounds depends upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disease, disorder, or condition, the condition of the patient, and the actual choice of compounds used. In certain instances, the determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a N-linked glycan inhibitor described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve remission (partial or complete) of a cancer, whereupon the therapeutic agent described herein (e.g., any N-linked glycan inhibitor) is subsequently administered. In addition, the combination methods, compositions and formulations are not to be limited to the use of only two agents; the use of multiple therapeutic combinations are also envisioned (including two or more therapeutic compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In addition, the N-linked glycan inhibitors described herein also are optionally used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a gene or gene mutation that is known to be correlated with certain diseases or conditions.

In various embodiments, the N-linked glycan inhibitors described herein and combination therapies are administered before, during or after the occurrence of a disease or condition. Timing of administering the composition containing a N-linked glycan inhibitor is optionally varied to suit the needs of the individual treated. Thus, in certain embodiments, the N-linked glycan inhibitors are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The administration of the N-linked glycan inhibitors are optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from about 1 month to about 3 months. The length of treatment is optionally varied for each subject based on known criteria. In exemplary embodiments, the compound or a formulation containing the compound is administered for at least 2 weeks, between about 1 month to about 5 years, or from about 1 month to about 3 years.

In certain embodiments, therapeutic agents are combined with or utilized in combination with one or more of the following therapeutic agents in any combination: immunosuppressants or anti-cancer therapies (e.g., radiation, surgery or anti-cancer agents).

In some embodiments, one or more of the anti-cancer agents are proapoptotic agents. Examples of anti-cancer agents include, by way of non-limiting example: gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARR^{y}-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L2), interferon alfa-2a; interferon alfa-2b; interferon alfa-n1; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sd±1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that are used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD20 and/or a CHOP (cyclophosphamide, doxorubicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

A pharmaceutical composition, as used herein, refers to a mixture of a N-linked glycan inhibitor described herein, with other chemical components, such as carriers, stabilizers, diluents, dispersing agents, suspending agents, thickening agents, and/or excipients. In certain instances, the pharmaceutical composition facilitates administration of the N-linked glycan inhibitor to an individual or cell. In certain embodiments of practicing the methods of treatment or use provided herein, therapeutically effective amounts of N-linked glycan inhibitors described herein are administered in a pharmaceutical composition to an individual having a disease, disorder, or condition to be treated. In some embodiments, the individual is a human. As discussed herein, the N-linked glycan inhibitors described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein are optionally manufactured in a conventional manner, such as, by way of example only, by means of conventional mixing, dissolving, granulating, dragee-making, levigating, emulsifying, encapsulating, entrapping or compression processes.

In certain embodiments, a pharmaceutical compositions described herein includes one or more N-linked glycan inhibitor described herein, as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the N-linked glycan inhibitors presented herein are also considered to be disclosed herein.

A “carrier” includes, in some embodiments, a pharmaceutically acceptable excipient and is selected on the basis of compatibility with N-linked glycan inhibitors disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein is formulated as a dosage form. As such, in some embodiments, provided herein is a dosage form comprising a N-linked glycan inhibitor described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of an N-linked glycan inhibitor. In one embodiment, a N-linked glycan inhibitor described herein is in the form of a particle and some or all of the particles of the compound are coated. In certain embodiments, some or all of the particles of a N-linked glycan inhibitor described herein are microencapsulated. In some embodiment, the particles of the N-linked glycan inhibitor described herein are not microencapsulated and are uncoated.

In certain embodiments, the pharmaceutical composition described herein is in unit dosage forms suitable for single administration of precise dosages. In unit dosage form, the formulation is divided into unit doses containing appropriate quantities of one or more therapeutic compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

### EXAMPLES

### Example 1

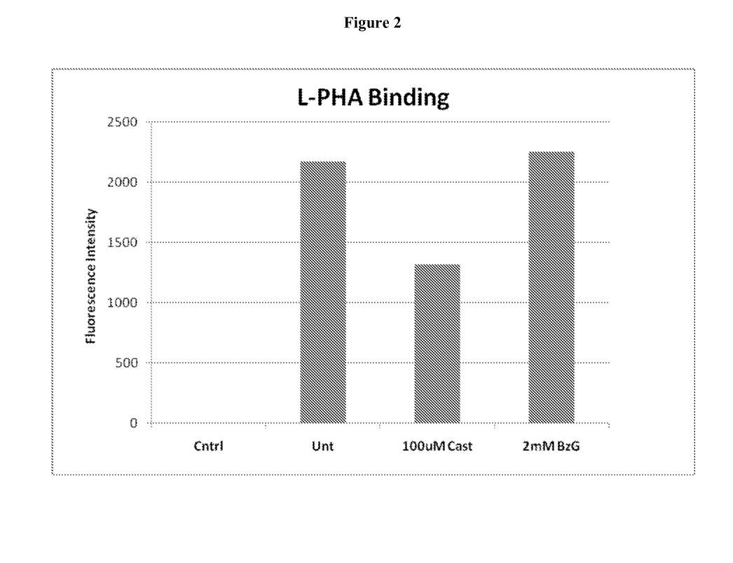

Fluorescein labeled L-PHA (FL-111, Vector Labs) is used. Chinese Hamster Ovary (CHO) cells were grown in the absence (Unt) or in the presence of 100 uM castanospermine (Cast) which blocks the initial processing of N-linked glycans or 2 mM benzyl-α-N-acetylgalactosamine (BzG) which inhibits the formation of O-glycans. Cntrl samples contained no lectin. L-PHA was biotinylated and identified using PE-Cy5 Strepavidin (BD Pharmingen). FIG. 2 shows Flow cytometry showing specific binding of the lectin Phaseolus vulgaris Lekoagglutinin (L-PHA) which binds to complex-type N-glycans with α1-6 mannose substituted branches.

### Example 2

### Primary Assay

The impact of an N-linked glycan synthesis inhibitor on the ability of a protein (e.g. integrin) to bind to N-linked glycans in mammalian cells is tested by incubating mammalian cells in the presence of an N-linked glycan synthesis modulator. After a suitable period of growth, the cells are released with 5 mM EDTA and probed with a labeled probe (biotinylated probe) for 30 minutes on ice. After washing unbound probe, the bound probe is detected with a suitable assay (e.g., tagging with streptavidin-Cy5-PE). After washing, the bound probe is quantified using flow cytometry. The N-linked glycan synthesis inhibitors are tested on at least three independent occasions, in duplicate over a dose range.

### Secondary Assay

N-linked glycan specificity is then determined by probing with lectins and/or proteins that bind to other glycan classes (chondroitin sulfate, Heparan sulfate, O-linked, etc.).

### Determination of Composition of N-Linked Glycans

Free glycans from the peptide backbone are obtained by hydrazinolysis which involves reacting with hydrazine (hydrazinolysis), acetylating with acetic anhydride/sodium bicarbonate, acidification and purification of the free glycans. Preferential release of N-linked glycans is selected by altering the conditions (time and temperature) of the hydrazinolysis reaction. Typically, the released glycans are labeled e.g. with the fluorescent tag, 2-aminobenzamide (2-AB) by reductive amination. Glycan structures can then be analyzed by HPLC (e.g. Glycoprep N column, Oxford Glycosciences) using a buffer gradient and fluorescence detection. The composition and sequence of the glycans can be further analyzed by digestion at specific monosaccharide residues with one or a combination of specific glycosidases e.g. sialic acid (A. ureafaciens sialidase), galactose (S. pneumoniae β-galactosidase), fucose (bovine epididymis α-fucosidase), N-acetylhexosamine (jackbean β-N-acetylhexosaminidase), N-acetylglucosamine (S. pneumoniae N-acetyl-β-D-glucosaminidase), mannose (jackbean α-mannnosidase) and internal galactose (B. fragilis endo-β-galactosidase). Following digestion the glycans are reanalyzed by HPLC.

Unlabeled glycans are analyzed by mass spectrometry (MS). In addition, sialic acid residues are esterified. Neutral (digested as outlined above) and sialic acid methyl ester containing oligosaccharides are analyzed by MS including MALDI MS on an instrument externally calibrated with a mixture of dextran oligomers.

Another method for analyzing glycans on glycoproteins involves removing N-linked glycans from the polypeptide with the enzyme Peptide: N-Glycosidase F, also known as PNGase F. In the procedure, cell or tissue material is extracted with detergent. Then it is reduced, carboxymethylated, digested with trypsin and the glycopeptides purified by reverse phase C18 column chromatography. N-linked glycans are released from the peptides with PNGase F. The N-linked glycans are cleaned up with a reverse phase C18 column chromatography (C18 Sep-Pak cartridge). For MS analysis, the purified glycans are permethylated and can be analyzed by various techniques including matrix-assisted laser desorption ionization “time-of-flight” (MALDI-TOF) and collisionally activated dissociation electrospray tandem mass spectrometry (CAD-ES-MS/MS). For linkage analyses the permethylated glycans are hydrolyzed, reduced, acetylated and analyzed by gas chromatography mass spectrometry (GC-MS).

### Example 3

### Affects of Modulators on the Ability of the Lectin Phaseolus vulgaris Agglutinin Type L (PHA) to Bind to Treated and Untreated Chinese Hamster Ovary (CHO) Cells

PHA binds to tri- and tetra-antennary complex-type N-glycans containing α1-6 mannose residues substituted at C-2 and C-6 with lactosaminyl disaccharides. Cultured CHO cells were treated with and without the test compound. After 2 days of growth the cells were released with 5 mM EDTA/PBS and probed with PHA for 1 hour on ice. After washing to remove unbound PHA, PHA was detected with streptavidin-Cy5-PE. After washing to remove the unbound streptavidin-Cy5-PE the bound probe was quantified using flow cytometry. Compound doses are in uM. The Y-axis shows the fluorescence intensity from the flow cytometer. The test compounds were tested on at least 3 independent occasions in duplicate over a dose range. FIGS. 10-16 illustrate that N-linked glycan biosynthesis inhibitors described herein demonstrates dose dependent reduction of PHA binding which requires complex N-linked glycans with a β1,6 linked GlcNAc branch.

### Example 4

### The Specificity of N-Linked Glycan Inhibitors was Determined by Probing with PHA and with Fibroblast Growth Factor 2 (FGF2)

PHA binds to tri- and tetra-antennary complex-type N-glycans containing α1-6 mannose residues substituted at C-2 and C-6 with lactosaminyl disaccharides. FGF2 is specific for another class of glycans (heparan sulfate). Cultured Chinese Hamster Ovary (CHO) cells were treated with and without the test compound. After 2 days of growth the cells were released with 5 mM EDTA. Parallel cultures were then probed with either PHA or FGF2 for 1 hour on ice. After washing to remove unbound lectin, bound lectins were detected with streptavidin-Cy5-PE. After washing to remove the unbound streptavidin-Cy5-PE the bound probes were quantified (separately) using flow cytometry. Compound doses are in uM. The Y-axis shows the % binding relative to the untreated cells. The test compounds were tested on at least 3 independent occasions in duplicate over a dose range. FIGS. 11-22 illustrate that N-linked glycan synthesis inhibitors according to certain embodiments herein show selective inhibition of N-linked glycans without inhibiting other unrelated glycans, such the GAG heparan sulfate. Similarly, FIGS. 31A-31T illustrate various compounds that selectively inhibit N-linked glycans over GAGs, such as heparan sulfate, in a similar manner.

### Example 5

### Effects of Inhibitors on Specific N-Linked Glycan Peaks

N-linked glycans were purified from CHO cell cultures and different N-linked glycan structural peaks were separated by normal phase HPLC. For the analyses, cultured CHO cells were treated with and without the test compounds as described above and then harvested for N-glycan profiling. The spent medium was decanted and the cells were rinsed 3 times with PBS and detached with 5 mM EDTA/PBS. N-glycans were released from cells using PNGaseF (Prozyme, Cat# GKE-5006) as described by the manufacture. Released glycans were labeled with 2-Aminobenzamidine (2AB) (Sigma, Cat# A89804) as described by the manufacture. Non-incorporated 2AB was removed using a Discovery DPA-6S column. Briefly, the column was pre-equilibrated 2× with 1 ml 97% ACN. Samples were loaded by adding 1 ml 97% acetonitrile (ACN) to the reaction mix. The column was washed 4× with 1 ml 97% CAN. 2AB-labeled N-glycans were then eluted 2× with 0.6 ml water, dried in a vacuum centrifuge, resuspended and then analyzed by normal phase HPLC(NP-HPLC). Analogous experiments were run with Castanospermine for comparison. The results are illustrated in FIGS. 23-30.

Purified 2AB-N-glycans were separated (NP-HPLC) on a 4.6×250 mm TSK Gel-Amide-80 column (Tosoh Bioscience). A gradient was run from 70% A to 50% A over 40 min at a flow rate of 1.2 ml/min (Solvent A: Acetonitrile, Solvent B: 50 mM ammonium formate pH4.4). Glucose units were determined based on 2AB-labeled glucose oligomer ladder (Prozyme). 2AB-labeled N-glycan standards (Prozyme) were run and assigned glucose units.

While preferred embodiments of the present invention have been shown and described herein, such embodiments are provided by way of example only. Various alternatives to the embodiments described herein are optionally employed in practicing the inventions. It is intended that the following claims define the scope of the invention and that methods and structures within the scope of these claims and their equivalents be covered thereby.

1. A process for modifying the population of N-linked glycans on one or more proteins associated with a cell, the process comprising contacting a cell that produces N-linked glycans with an effective amount of a selective late stage N-linked glycan biosynthesis inhibitor, the selective late stage N-linked glycan biosynthesis inhibitor being active in a mammalian cell.

2. The process of claim 1, wherein the selective late stage N-linked glycan biosynthesis inhibitor is a non-carbohydrate inhibitor.

3. The process of claim 1, wherein the selective N-linked glycan biosynthesis inhibitor has a molecular weight of less than 700 g/mol.

4. The process of claim 1, wherein the process reduces the ratio of complex N-linked glycans to high mannose N-linked glycans.

5. The process of claim 1, wherein the process reduces the amount of tri-antennary and tetra-antennary N-linked glycans in the cellular population of N-linked glycan.

6. The process of claim 1, wherein the process reduces the cellular population of (β1-6) branching N-linked glycans.

7. The process of claim 1, wherein the process reduces the cellular population of poly-N-acetyllactosamine-containing N-glycans.

8. The process of claim 1, wherein the process reduces the cellular population of outer-chain polyfucosylation, sialyl Lewis X containing N-glycans, or both.

9. The process of claim 1, wherein the selective N-linked glycan biosynthesis inhibitor inhibits GlcNAc-T-V, GlcNAc-T-IV, GlcNAc-T-III, GlcNAc-T-II, or a combination thereof.

10. The process of claim 9, wherein the selective N-linked glycan biosynthesis inhibitor indirectly inhibits GlcNAc-T-V, GlcNAc-TIV GlcNAc-T-III, GlcNAc-T-II, or a combination thereof.

11. The process of claim 9, wherein the selective N-linked glycan biosynthesis inhibitor directly inhibits GlcNAc-T-V, GlcNAc-T-IV, GlcNAc-T-111, GlcNAc-T-11, or a combination thereof.

12. The process of claim 1, wherein the selective N-linked glycan biosynthesis inhibitor inhibits modifications including (α2,3) sialylation, (α2,6) sialylation, (α1,3) fucosylation, 6-sulfation of the terminal galactose, 6-sulfation of the penultimate GlcNAc.

13. The process of claim 1, wherein the cell is an inflammatory cell, a cancer cell, an endothelial cell, a cell having abnormal N-linked glycan accumulation, or a cell susceptible to viral and pathogenic infection.

14. The process of claim 1, wherein the cell is present in an individual diagnosed with or suspected of having rheumatoid arthritis, Crohn's disease, inflammatory bowel disease, lung cancer, colon cancer, breast cancer, pathogenic angiogenesis, or diabetes.

15. The process of claim 1, wherein the cell is present in an individual diagnosed with or suspected of having influenza, HIV, lysosomal storage disease, sialidosis, or fucosidosis.

16. An N-linked glycanated protein comprising a core protein covalently linked to at least one N-linked glycan, (i) the at least one N-linked glycan comprising a plurality of high mannose, hybrid or complex N-linked glycan structures, and (ii) less than 9 mol % of the plurality of high mannose, hybrid or complex N-linked glycan structures being tri-antennary N-linked glycans, less than 2 mol % of the plurality of high mannose, hybrid or complex N-linked glycan structures being tetra-antennary N-linked glucans, or both.

17. (canceled)

18. An N-linked glycanated protein comprising human serum acid alpha-1-glycoprotein N-linked glycanated comprising bi-antennary, tri-antennary and tetra-antennary N-linked glycans, wherein less than 52% (w/w) of the N-linked glycans are the sum of the tri-antennary and tetra-antennary N-linked glycans, less than 12% (w/w) of the N-linked glycans are tetra-antennary, or both.

19. (canceled)
